(19) United States
(12) Patent Application Publication
(10) Pub. No.: US 2022/0018915 A1
(43) Pub. Date: Jan. 20, 2022

(54) SYSTEMS AND METHODS FOR GENERATION OF HYPERPOLARIZED MATERIALS

(71) Applicant: Nvision Imaging Technologies GmbH, Blaustein (DE)

(72) Inventors: Ilai SCHWARTZ, Ulm (DE); Tim Rolf EICHHORN, Blaustein (DE); Christophoros VASSILIOU, Ulm (DE); Michael KEIM, Blaustein (DE)

(73) Assignee: Nvision Imaging Technologies GmbH, Blaustein (DE)

(21) Appl. No.: 17/309,345

(22) PCT Filed: Nov. 20, 2019
(86) PCT No.: PCT/IB2019/001316

§ 371 Date: May 20, 2021

Related U.S. Application Data
(60) Provisional application No. 62/867,676, filed on Jun. 27, 2019.
(60) Provisional application No. 62/777,173, filed on Dec. 9, 2018.
(60) Provisional application No. 62/770,276, filed on Nov. 21, 2018.

(51) Int. Cl. G01R 33/28; G01N 24/08

(52) U.S. Cl. CPC G01R 33/282; G01N 24/08

(57) ABSTRACT

Systems and methods for generating hyperpolarized target materials are disclosed. The disclosed systems and methods can include hyperpolarizing a compound then transferring polarization to a target material. The compound can be selected to have nuclear spins. The compound can be further selected to have electron spins that, when exposed to certain electromagnetic radiation, exceed a predetermined level of polarization. The compound can be exposed to such electromagnetic radiation, optically hyperpolarizing the electron spins of the compound. Polarization can then be transferred from the electron spins of the compound to nuclear spins of the compound, at least in part by exposing the compound to a magnetic field. The compound can be exposed to the target material before or after pulverizing the compound to increase the surface area of the compound, thereby facilitating transfer of polarization from the compound to the target material.

### CROSS REFERENCE TO RELATED APPLICATIONS

This application claims the benefit of priority of U.S. Provisional Patent Application No. 62/770,276 filed Nov. 21, 2018, U.S. Provisional Patent Application No. 62/777,173 filed Dec. 9, 2018, and U.S. Provisional Patent Application No. 62/867,676 filed Jun. 27, 2019, the contents of each of which are incorporated by reference in their entirety.

### TECHNICAL FIELD

The disclosed embodiments generally relate to generation of hyperpolarized materials for use in nuclear magnetic resonance, magnetic resonance imaging, or similar applications.

### BACKGROUND

Nuclear magnetic resonance (NMR) and magnetic resonance imaging (MRI) are technologies with vital applications in chemistry, biology and medical imaging. Despite these successes, it is recognized that nuclear magnetic resonance applications have limitations due to the minute nuclear polarization of analytes (typically on the order of 10⁻⁵). This minute nuclear polarization can result in limited sensitivity in comparison to other analytic techniques such as mass spectrometry.

Increasing nuclear spin polarization beyond its thermal equilibrium value can improve magnetic resonance sensitivity. Nuclear spin polarization can be increased using known techniques like dynamic nuclear polarization. Using such techniques, the nuclear spin polarization of a material can be increased 10,000 times or more. The enhanced nuclear spin polarization can result in a proportional increase in the NMR/MRI signal. While this enhanced polarization decays over time due to the relaxation time of the nuclear spins in the polarized molecules, for many molecules the relaxation time can be on the order of seconds to minutes, during which increased polarization can lead to a dramatic increase in NMR/MRI signal sensitivity. By enabling such a dramatic increase in NMR/MRI signal sensitivity, increased nuclear spin polarization can enable new applications, such as the imaging of in vivo metabolism using metabolites with increased nuclear spin polarization in an MRI scanner, accelerate signal NMR spectroscopy investigations, and enable visualization of previously unseen molecular dynamics and structures

### SUMMARY

In accordance with the present disclosure, a method is provided for forming a target material. The target material can be a hyperpolarized NMR or MRI target material. The method can include multiple operations. The operations can include obtaining a compound having nuclear spins. The compound can be selected to have, under optical radiation, electron spins exceeding 10% polarization. The operation can further include optically hyperpolarizing electron spins of the compound. The operation can further include transferring polarization from the electron spins of the compound to nuclear spins of the compound, at least in part, by exposing the compound to a magnetic field. The operation can further include exposing the compound to a target material before or after pulverizing the compound to increase the surface area of the compound, thereby facilitating transfer of polarization from the compound to the target material.

Further in accordance with the present disclosure, a method is provided having multiple operation. The operation can include forming a mixture of a compound and a target material. The operation can further include performing at least one iteration of polarization transfer. The one iteration can include: polarizing nuclear spins of a species in the compound. The one iteration can further include transferring the nuclear spin polarization of the compound to nuclear spins of the target material.

Further in accordance with the present disclosure, a method is provided for polarization. The method can have multiple operations. The operation can include forming a mixture of a compound and a target material. The compound includes a dopant selected to have, under optical radiation, electron spins exceeding 10% polarization. The at least one of the compound or the target material can be in a form of a nanostructure. Nuclear spins of the compound can be polarized at a level of more than 0.1% polarization. The operation can further include transferring polarization of the nuclear spins of the compound to the target material.

Further in accordance with the present disclosure, there is provided a system. The system can include a first housing containing. The system can further include a first cavity configured to hold a pulverized compound with pre-polarized nuclear spins. The system can further include a mixing apparatus configured to mix the pulverized compound into a mixture. The system can further include a first magnetic field generator configurable to maintain a magnetic field of at least 10 gauss within a predetermined portion of the first cavity during the mixing of the pulverized compound into the mixture.

Further in accordance with the present disclosure, a method is provided having multiple operation. The operations include introducing into a first cavity a pulverized compound with pre-polarized nuclear spins. The operation can further include mixing the pulverized compound into a mixture. A magnetic field of at least 10 gauss can be maintained within the first cavity during the mixing of the pulverized compound into the mixture.

Further in accordance with the present disclosure, a method is provided for preparing a target material. The method can include multiple operations. The operations can include introducing into a cavity, a compound with pre-polarized nuclear spins. The operations can further include introducing into the cavity, material comprising a solvent or a combination of a solvent and target material. The operations can further include pulverizing the compound. The pulverized compound includes pieces having a median size of no greater than 1 mm³. The operations can further include mixing the pulverized compound and the materials into a mixture. The temperature of the cavity can be maintained at less than −20 degree C. and a magnetic field of at least 10 gauss can be applied to the cavity during the pulverizing and mixing of the compound. The operations can further include polarizing the mixture for a predetermined duration by applying to the mixture, in the cavity for a predetermined duration, two or more electromagnetic fields at two or more frequencies that excite nuclear spins in the mixture, and a magnetic field of at least 10 gauss having inhomogeneities of at most ±20% within a predetermined portion of the fourth cavity. The operations can further include conveying the mixture through a location within 1 second. A magnetic field at the location can be less than 300 gauss during the conveying of the sample through the location. The operations can further include introducing a second solvent having a temperature greater than 0 degree C. into the cavity having, thereby dissolving from the mixture the target material. The operations can further include extracting the target material from the cavity.

Further in accordance with the present disclosure, a method is provided for forming an NMR or MRI target material. The method can include multiple operations. The operation obtaining at least 0.1 mg of a compound containing nuclear spins. The nuclear spins in the compound can exceed 0.1% polarization. The operations can further include exposing the compound to a target material. The operations can further include mechanically altering the compound to increase a surface area of the compound and facilitate transfer of polarization from the compound to the target material.

Further in accordance with the present disclosure, a method is provided for transferring polarization. The method can include multiple operations. The operations can include hyperpolarizing a compound at a first location, the hyperpolarized compound having a relaxation time greater than 2.5 hours when maintained at a temperature between 70 and 273 Kelvin in a magnetic field of a strength between 0.05 and 4 Tesla. The operations can further include transporting the hyperpolarized compound to a second location in a container configured to maintain the hyperpolarized compound at the temperature in the magnetic field strength. The operations can further include transferring polarization from the compound to a target material at the second location.

Further in accordance with the present disclosure, there is provided a container. The container can include a refrigerant. The container can further include a magnetic field source. The container can further include a cryostat containing a hyperpolarized compound having a relaxation time greater than 2.5 hours when maintained at a temperature between 70 and 273 Kelvin in a magnetic field of a strength between 0.1 and 4 Tesla. The container can be configured to maintain the hyperpolarized compound at the temperature in the magnetic field using the refrigerant and the magnetic field source.

Further in accordance with the present disclosure, a method is provided for manufacturing a hyperpolarized biocompatible material. The method can include multiple operations. The operations can include mixing the hyperpolarized biocompatible material with a non-biocompatible material containing nuclear spins into a mixture. The non-biocompatible material can include a dopant with hyperpolarizable electron spins. The operations can further include optically hyperpolarizing the electron spins of the dopant. The operations can further include transferring polarization from the electron spins of the dopant to the nuclear spins of the non-biocompatible material. The operations can further include transferring polarization of the nuclear spins of the non-biocompatible material to nuclear spins of the biocompatible material. The operations can further include preparing a second mixture of the biocompatible material for injection into biological tissue at least in part by separating the second mixture from the first mixture. The second mixture can include at least some of the biocompatible material from the first mixture and having a concentration of less than 1 mM of the non-biocompatible material from the first mixture.

Further in accordance with the present disclosure, a method is provided for forming an NMR or MRI target material. The method can include multiple operations. The operations can include obtaining at least 0.1 mg of a compound containing nuclear spins. The compound can be hyperpolarized at a level of more than 0.1% polarization. The operations can further include creating a mixture containing the compound and a target material by dissolving the compound in a solution. The operations can further include freezing the mixture of the solution and the target material within a predetermined time from the beginning of the mixing of the compound and target material.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 depicts an exemplary process for generating polarized target materials, consistent with disclosed embodiments.

FIGS. 2A and 2B depict polarization of a triplet state population using photoexcitation, consistent with disclosed embodiments.

FIG. 3A depicts incorporation of pentacene dopants into naphthalene crystals, consistent with disclosed embodiments.

FIG. 3B depicts a naphthalene crystal doped with pentacene, consistent with disclosed embodiments.

FIGS. 4A and 4B depict spin transference for an exemplary DNP method that achieves spin transfer using the Solid Effect, consistent with disclosed embodiments.

FIG. 5 depicts an exemplary sequence of optical irradiation, magnetic field sweep and electromagnetic irradiation suitable for inducing polarization in a compound, consistent with disclosed embodiments.

FIG. 6 depicts an NMR signal reads from a compound before and after repeated iterations of the polarization sequence depicted in FIG. 5, consistent with disclosed embodiments.

FIG. 7 depicts an exemplary decrease in polarization of a compound over time, consistent with disclosed embodiments.

FIGS. 8A and 8B depict exemplary polarization time dependencies for a compound before pulverization and after pulverization, consistent with disclosed embodiments.

FIG. 8C depicts an exemplary polarization time dependence for a pulverized compound at two different temperatures, consistent with disclosed embodiments.

FIGS. 9A and 9B depict exemplary scanning electron microscope (SEM) and optical microscope images of a pulverized naphthalene sample, consistent with disclosed embodiments.

FIG. 10 depicts an exemplary mixture of a pulverized solid compound and a target material in solution, consistent with disclosed embodiments.

FIG. 11 depicts the exemplary addition of a liquid mediator to a mixture of a pulverized compound and a pulverized target material, consistent with disclosed embodiments.

FIGS. 12A to 12D depict views of an exemplary apparatus for polarizing a compound, consistent with disclosed embodiments.

FIG. 13 describes an exemplary transport device, consistent with disclosed embodiments.

FIGS. 14A to 14E depict exemplary components collectively capable of transferring polarization from a polarized compound to a target material and separating the compound and target material, consistent with disclosed embodiments.

FIGS. 15A to 15C depict views of an exemplary polarization transfer system, consistent with disclosed embodiments.

FIGS. 16A and 16B depict views of an alternative exemplary polarization transfer system, consistent with disclosed embodiments.

FIG. 17 depicts an exemplary process in which the compound is mixed with the target material prior to polarization, consistent with disclosed embodiments.

FIGS. 18A and 18B depict an exemplary preparation of a target material entrapped in polycrystals of a compound, consistent with disclosed embodiments.

FIGS. 19A and 19B depict an exemplary preparation of a target material entrapped in a single crystal, or a mostly single crystal, of the compound, consistent with disclosed embodiments.

FIGS. 20A to 20E depict an exemplary process of polarization diffusion, consistent with disclosed embodiments.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Conventional methods of nuclear polarization, such as dynamic nuclear polarization (DNP) can use magnetic fields and electromagnetic radiation to produce polarized target materials. For example, dissolution dynamic nuclear polarization (dDNP) can be used to produce highly polarized target materials, such as metabolites and other relevant NMR molecules. However, temperature, timescales, and magnetic fields constraints have made dDNP a technically challenging endeavor. In particular, dDNP can require polarizers that can operate at ˜1K temperatures and >4 T magnetic fields. Such polarizers must be placed near the point of use of the target materials (e.g., the MRI suite) to minimize polarization loss in the target materials during transport. Other DNP methods, and other methods of nuclear polarization can have similarly restrictive temperature and magnetic field strength requirements.

The disclosed embodiments provide a technical solution to the temperature, timescales, and magnetic fields constraints of conventional methods of nuclear polarization. In particular, the disclosed embodiments include a novel hyperpolarization method, which separates a technically challenging first step of hyperpolarization from a second step of transferring the polarization to the target material for hyperpolarized MM/NMR. In the first step, a material, which has a long polarization relaxation time and is optimized for polarization, is polarized in a polarizer, which can be spatially separated from the MRI suite. The polarized material can then be transported while being kept in a magnetic field and cold temperatures to a second system in the vicinity of the MRI suite while maintaining polarization. At the second system, the polarized material can be used as a polarization source material (e.g., nuclear spin polarization can be transferred from the nuclear spins of the polarization source to the nuclear spins of a target material). In preferred embodiments, the transfer can be preceded by a step of increasing the surface area of the polarized material (e.g., by pulverizing the polarized material). The polarized material can then be mixed with the target material, which can enable polarization to diffuse or be actively transferred to the target material. In a preferred embodiment, the target material is then extracted and used as an agent in hyperpolarized MRI or NMR operations.

“Polarization” can include an imbalance in electron or nuclear spins orientations. In some embodiments, polarization can be the normalized, approximate difference in the number of spins in a first direction minus a number of spins in the opposite direction. As a non-limiting example, given 200,000 1H nuclear spins, a polarization of 2% can correspond to 102,000 spins in the first direction and 98,000 in the opposite direction. In some embodiments, “hyperpolarization” can include polarization of a species (e.g., nuclear, election, or the like) in excess of typical polarization levels for that species observed at thermal equilibrium subject to exposure to a specified magnetic field. As a non-limiting example, a sample in a 1 T magnetic field at thermal equilibrium, with 1H nuclear spin polarization in excess of 0.000341% can be hyperpolarized. As an additional nonlimiting example, a sample in an 3 T magnetic field at thermal equilibrium, with 13 C spin polarization in excess of 0.000257% can be hyperpolarized. As a further nonlimiting example, a sample in an 3 T magnetic field at thermal equilibrium, with 15N spin polarization in excess of 0.000103% can be hyperpolarized.

A “polarizable material” or material containing “polarizable molecules” can contain molecules that, when exposed to suitable optical radiation, have electron spins exceeding 0.1% polarization, 1% polarization, 10% polarization, 30% polarization, or 80% polarization. In some embodiments, the polarizable material can have triplet spin states. In some embodiments, the suitable optical irradiation can induce electron polarization by initial selective population of the triplet spin states. In various embodiments, suitable optical radiation can induce electron polarization through differential decay rates in the triplet spin states. In some embodiments, the suitable optical irradiation can induce electron polarization through a combination of an inversion pulse between triplet states following the optical irradiation and differential decay rates.

A PETS (Photoexcited triplet states) material can include polarization molecules. In some embodiments, the nuclear spins of a PETS material are suitable for polarization using spin-polarized electron triplet states of the PETS material. The spin-polarized photoexcited electron triplet states can provide on demand electron polarization at a wide range of magnetic fields and temperatures, even combinations where the thermal electron polarization is orders of magnitude below unity. Moreover, the photo-excitable triplet states can have a singlet ground level to which they will decay to. Accordingly, whenever the electron is not excited to an excited state, it is not a paramagnetic center and does not cause relaxation. A PETS material can therefore have a nuclear relaxation time at liquid nitrogen temperatures or above, in the absence of optical irradiation, of over an hour, 10 hours, or 50 hours. In some embodiments, a PETS material can include a combination of a polarizable material and a host material. The host material may be, for example naphthalene.

A “compound” can be a polarized material which is used to transfer polarization to another material (e.g., a target material). Following polarization, as described herein, the polarization of the compound can be greater than 0.1%, greater than 1%, greater than 10%, greater than 30%, greater than 50%, or greater. The compound can be present in a solid, liquid, or gas form. In some embodiments, the solid compound can have a crystalline or amorphous structure. In various embodiments, the compound can be a powder, such as a powder formed from crystalline or amorphous structures (e.g., micro- or nano-crystals, or polycrystals). The compound can be or can include a PETS material. In some embodiments, the polarized nuclear spins in the compound can have a relaxation time longer than 1 minute, 10 minutes, 1 hour, 10 hours or 100 hours at a magnetic field lower than 15 T or lower than 1 T and a temperature higher than 1K, 4K, or 70K. In some embodiments, the compound can be modified (e.g. by pulverization or dissolution), without losing a significant portion of its polarization. In various embodiments, the compound can have a large surface area (e.g., the compound can be porous). In some embodiments, following polarization, a compound can incorporate less than 1000 ppm paramagnetic impurities, less than 100 ppm, or less than 1 ppm.

In some embodiments, the compound can contain a trace amounts of paramagnetic impurities. Such a compound can be a bio-compatible molecular crystal (e.g., water ice, urea crystals, fumarate crystals, or the like). In some embodiments, the compound can contain significant amounts of paramagnetic impurities (e.g., more than 10 ppm, 100 ppm, or 1000 ppm). Such paramagnetic impurities can enable the polarization of the compound by dynamic nuclear polarization (DNP), typically resulting in higher polarization than achievable in compounds with trace amounts of paramagnetic impurities. In certain embodiments, some paramagnetic impurities can be optically hyperpolarized, for example diamonds with nitrogen-vacancy defects or silicon-carbide with silicon-vacancy or divacancy defects. In other embodiments the compound can include thermally polarized paramagnetic impurities (e.g., crystals with defects caused by irradiation with electrons or ions, glassy substrates containing radicals with free electron spins, or the like). In some embodiments, the electron spin concentration can be significantly reduced after polarizing the compound. For example, the compound can be composed of micro- or nano-particles and the electron spins used for the polarization can be in radicals in an external glassy matrix. The microparticles can be separated from the glassy matrix following the polarization. In another embodiment, radicals in the material are produced by UV irradiation. These radicals can advantageously be quenched when raising the temperature above a certain threshold, thereby increasing the relaxation time of the compound following polarization. In various embodiments, the compound can contain transient paramagnetic impurities (e.g., transient paramagnetic impurities can used for polarization and decay after the polarization).

In some embodiments, the compound can include a polarization molecule as a dopant, with the polarization molecule incorporated in concentrations lower than 2% mol/mol, more preferably lower than 0.2% mol/mol. In various embodiments, the compound can include a larger concentration of the polarization molecule, with the polarization molecules constituting at least 10% mol/mol of the compound, and potentially 50% mol/mol or more. Select examples of compounds where the polarization molecules consist of at least 10% mol/mol include benzene crystals, naphthalene crystals, pentacene crystals, cyclohexanone crystals, benzophenone crystals, testosterone crystals

Consistent with disclosed embodiments, polarization molecules usable as dopants in a compound (e.g., a host crystal) can include pentacene:naphthalene, pentacene:p-terphenyl, pentacene:benzoic acid, acridine:fluorene, acridine:biphenyl, diazapentacene:p-terphenyl, pyrene:benzene. In a non-limiting embodiment, the compound can be an aromatic hydrocarbon, and the polarization molecule can be a hydrocarbon molecule suitable for incorporated in the preferred amounts into the compound. However, other organic molecules are also possible. For example, a non-limiting list of polarization molecules and host crystals is disclosed in “Molecular spectroscopy of the triplet state through optical detection of zero-field magnetic resonance”, by Kinoshita et al. and expressly incorporated herein by reference.

In some embodiments, the compound can be a single crystal or an oriented Shpolsky matrix including the polarization molecules. In another embodiment, the compound can be a powder or polycrystal including the polarization molecules. The compound can contain at least one nuclear species with nuclear spins, which can be polarized by the electron spins of the polarization molecules. In some embodiments, the compound nuclear spins can exhibit a lengthy relaxation time. In some embodiments, the polarization molecules do not have a paramagnetic impurity after the electrons decay back to the singlet state S0, the compound can contain very small amounts of paramagnetic impurities, preferably less than 10000 ppm, more preferably less than 100 ppm, more preferably less than 1 ppm. In various embodiments, purification methods including zone refinement, re-sublimation, distillation, and the like can be used to enhance the purity of the compound. The resulting relaxation time of the relevant nuclear spins in the compound can be longer than 10 minutes at 77K and 0.5 T magnetic field, longer than an hour, longer than 10 hours, or longer than 100 hours.

A “target material” can be a polarizable material, or material containing polarizable molecules, to which polarization can be transferred. In some embodiments, after polarization transfer, the polarization of the polarizable material, or polarizable molecules, can be greater than 0.1%, greater than 1%, greater than 10%, greater than 30%, greater than 50%, or greater.

In some embodiments, the target material can be suitable for use in hyperpolarized MRI. When used in hyperpolarized MRI, the target material can greatly increase MRI signal and signal-to-noise ratio (SNR). In some embodiments, the target material can include biocompatible molecules for injection into tissue or in vivo. In some embodiments the target material can include molecules of one or more of: urea, pyruvic acid, pyruvates, fumarate, bicarbonate, dehydroascorbate, glutamine, acetate, alpha-ketoglutarate, dihydroxyacetone, acetoacetate, lactate, glucose, ascorbic acid, and zymonic acid. In some embodiments, the target material can have isotopic labeling (e.g., 13 C or 15N isotropic labeling, or the like). A non-limiting set of additional suitable target materials is disclosed in “Hyperpolarized 13C MRI: path to clinical translation in oncology” by Kurhanewicz, John, et al. and incorporated herein by reference.

In some embodiments, the target material can be suitable for use in solution NMR spectroscopy. For example, the target material can be a combination of any small or large molecules of interest for examination in NMR spectroscopy. In some embodiments, the target material are metabolites used in NMR metabolomics applications. In some embodiments, the target material is a protein, polymer, or other macromolecule. In some embodiments, the target material can be suitable for in-vitro probing of the metabolism of a cell culture or other biological tissue. In some embodiments, the target material can be a molecule suitable for subsequent hyperpolarized proton exchange with another molecule of interest. In some embodiments the target material can be used in an NMR probe to investigate a transient effect in which high signal enhancement due to hyperpolarization is needed, such proton exchange between water and biomolecules. In another example, the target material can be a powder of particles used in magic angle spinning NMR spectroscopy.

In some embodiments, the target material may be a powdered, polycrystal, or amorphous solid suitable for use in solid-state NMR spectrometry (e.g., magic angle spinning NMR (MAS-NMR)). In some embodiments, the target material is a solution, gel, tissue or soft solid investigated in high-resolution MAS NMR. In some embodiments, the target material may include a solution in which polarizable molecules or particles are dissolved or suspended.

A “porous” material can be a material including voids. In some embodiments, a ratio between the surface area of the voids in a quantity of a porous material and the surface area of the quantity of the porous material can be greater than 3, 10, 100, 1000, 10000, or 10000. Accessible voids are void space accessible from the enveloping surface of a quantity of the porous material (e.g., open cells as opposed to closed cells).

A “microparticle” can be a particle that is smaller than 200 μm, 20 μm, or 2 μm in at least one dimension (e.g., smaller than 200 μm in two or three dimensions). In some embodiments, a microparticle can be globular. In various embodiments, a microparticle can have a single dimension significantly greater than the other dimensions. For example, in some embodiments, a microparticle can be rod- or fiber-shaped. In such embodiments, the length of rod- or fiber-like microparticle can be between smaller than 1000 μm, 100 μm, or 10 μm. Similarly, a “nanoparticle” can be a particle that is smaller than 200 nm, 20 nm, or 2 nm in at least one dimension (e.g., smaller than 200 nm in two or three dimensions). In some embodiments, a nanoparticle can be globular. In various embodiments, a nanoparticle can have a single dimension significantly greater than the other dimensions. For example, in some embodiments, a nanoparticle can be rod- or fiber-shaped. In such embodiments, the length of a rod- or fiber-like nanoparticle can be between smaller than 1000 nm, 100 nm, or 10 nm. In some embodiments, micro- or nano-particles may be packed tightly, thereby creating a semi-polycrystalline structure. As used herein, unless otherwise specified, a “particle” can be a nanoparticle or microparticle. The semi-polycrystalline structure can be porous, with accessible voids.

Overview

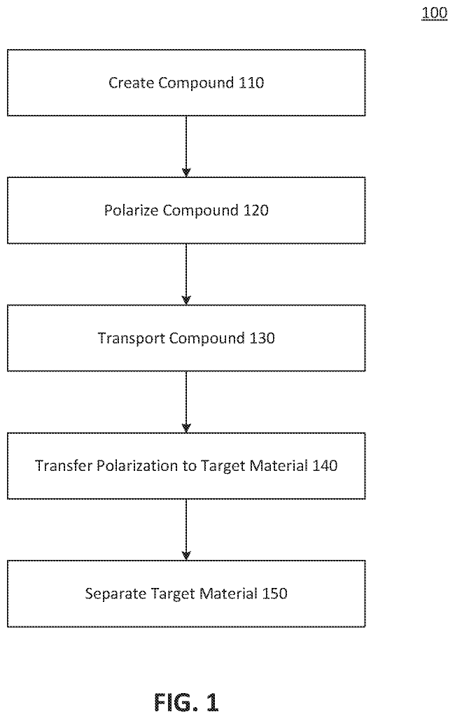

FIG. 1 depicts an exemplary process 100 for generating polarized target materials. In some instances, the polarized target material can be used as an agent in hyperpolarized MRI or NMR operations. Process 100 can include a step of obtaining a compound. The compound can be suitable for polarization and capable of retaining polarization for a long time under suitable conditions. The compound can be polarized, as described herein. Polarization can occur at an origin location. The compound can then be transported under conditions that cause it to retain polarization. Upon reaching a destination location, at least some of the polarization of the compound can be transferred to a target material. The polarization transfer can occur when the compound and the target material are brought into contact (e.g., combined into a mixture, solution, or the like) under suitable conditions. While the compound may be selected based on suitability for polarization and the ability to retain polarization for a long time under suitable conditions, the target material may be selected based on differing criteria. For example, the target material may be selected for suitability in an application, such as an imaging application. After transferring polarization to the target material, the polarized target material can then be separated from the compound. The polarized target material can then be used in the application. In this manner, polarization occurring at the origin location can be transferred to a target material at the destination location. The target material is therefore not limited to materials capable of retaining useful degrees of polarization during transit.

In step 110, process 100 begins by obtaining a compound having nuclear spins, as described above. The compound can be obtained by receiving the compound or creating the compound, as described herein. The compound can be a solid, a glassy matrix, a powder, an aggregate, or in another suitable form. The compound can be a crystalline compound. For example, the compound can be a single crystal solid or a multi-crystal solid. As additional example, the compound can be an aggregate of single or multi-crystal solids. For example, the compound can be a collection of microcrystals. The compound can include a base material and a dopant. In some embodiments, the compound can be a molecular crystal. The molecular crystal can be doped with the dopant. The dopant can be selected from a group of organic compounds. For example, the dopant can be aromatic hydrocarbons such as pentacene, pentacene derivatives, tetracene, tetracene derivatives, anthracene or anthracene derivatives. The dopant can enable the optical polarization of the compound.

In some embodiments, the base material and the dopant can be selected such that the compound has, under suitable optical radiation, electron spins exceeding 10% polarization. In various embodiments, the compound, when polarized, has a relaxation time greater than 2.5 hours when maintained at a temperature between 70 and 273 Kelvin in a magnetic field of a strength between 0.1 and 4 Tesla. In some embodiments, the compound, when polarized, has a relaxation time greater than 10 hours when maintained at a temperature between 70 and 150 K and the magnetic field strength is between 0.3-1 Tesla.

In step 120, the compound can be polarized. Polarization of the compound can include a step of polarizing electrons in the compound and a step of transferring polarized electron spins to nuclear spins of the compound. The steps may each be performed once or may alternate a predetermined number of times or a number of times sufficient to achieve a desired degree of polarization. In some embodiments, the polarized electrons can be intrinsic to the doping agent. In some embodiments, the electrons can be polarized optically.

Polarization can be transferred from the electron spins to the nuclear spins in the compound. Such transfer can be performed using dynamic nuclear polarization (DNP) protocols that use microwave or radio frequency irradiation, level avoided crossing (LAC) methods that use tuned the magnetic fields, or the like. In some embodiments, the DNP method can include exposing the compound to a magnetic field, as described herein. The magnetic field can be used to tuned the polarized electron spin to a particular resonance frequency. The resonance frequency can be common to an energy level of the optically polarized electrons and to nuclear spins of the compound. In some instances, the polarized electrons can be energized to transfer electron spin polarization to the nuclear spins of the compound using dynamic nuclear polarization methods including microwave or radiofrequency irradiation as described herein. The microwave energy can be provided at a frequency close to an electron paramagnetic resonance frequency of the polarized electrons. In embodiments where the electron spin is hyperpolarized, the transfer of the electron spin polarization can be performed in temperatures ranging from 4K to 500K and magnetic field strengths ranging from 1 mT to 20 T. In other embodiments relying on thermal polarization, the transfer of the electron spins can be performed a temperature below 80K, more preferably below 4K in a magnetic field higher than 3 T.

In some embodiments, the compound nuclear spins can be polarized at a level of more than 3000 times the thermal polarization level of the nuclear spins at room temperature and a 1 T magnetic field, which for 1H nuclear spins, in some embodiments, may correspond to more than 1% polarization. For example, the compound nuclear spins can be hyperpolarized to a level more than 30,000 greater than the thermal polarization level. In some embodiments, the nuclear spins can be spins of nuclei in the base material.

In step 130, the compound can be transported, in a container, from an origin location, where the polarization occurs, to a destination location. In some embodiments, the destination location can be more than a kilometer, more than 10 kilometers, more than 100 kilometers from the origin location, and a duration of the transportation can be greater than an hour, greater than 5 hours, greater than 10 hours.

The container can be configured to maintain the compound in a polarized state during transportation. The container can include a cavity for holding the compound, a temperature control system, a magnetic field source, a magnetic shield, and a control system for providing an alert in response to detecting an anomalous temperature or magnetic field strength. In some embodiments, the container can maintain a suitable environment for prolonging the relaxation time of the compound. For example, the container can be configured to maintain the compound within a magnetic field and at a temperature less than room temperature. As an additional example, the container can be configured to maintain the compound at a temperature between 70 and 273 Kelvin in a magnetic field of a strength between 0.1 and 4 Tesla. The container can be configured to maintain the hyperpolarized compound at the temperature in the magnetic field for more than an hour. In some embodiments, the relaxation time of the hyperpolarized compound is greater than 5 hours when maintained at the temperature in the magnetic field of the shipping container.

In some embodiments, the temperature control system can include a liquid (e.g., liquid nitrogen or the like) or solid (e.g., solid carbon dioxide or the like) refrigerant. The shipping container can include a cavity for containing the compound. For example, the compound can be disposed cryostat (e.g., a Dewar, vacuum flask, or the like) within the container. The container can be or can include a cryogenic dry-shipping container.

In some embodiments, the magnetic field source of the shipping container can be a permanent magnet or an electromagnet. The magnetic field source can generate a magnetic field with a strength between 0.1 and 4 Tesla. In some embodiments, the magnetic shield can be configured to substantially contain the magnetic field within the shipping container.

In some embodiments, the control system can include a processor and memory containing instructions for evaluating the temperature and magnetic field strength within the shipping container. The control system can be configured to receive information generated by one or more sensors. The sensors can include magnetometers and thermometers of known design. The particular sensors and their configurations are not intended to be limiting. Using the information received from the sensors, the control system can be configured to automatically monitor the magnetic field and the temperature (e.g., during transportation of the compound). In some embodiments, the control system can be configured to provide an alert in response to identifying an anomalous temperature or magnetic field strength. For example, should the detected temperature or magnetic field strength fall outside a predetermined range, the control system can be configured to provide the alert. The alert can include an audible or visual alert (e.g., a buzzer, flashing light, or the like). In some embodiments, the control system can be configured to provide a message to another computing system indicating the anomalous temperature or magnetic field strength (e.g., an email, SMS message, page, automated phone call, or the like).

In step 140, polarization is transferred from the compound to a target material. In some embodiments, the process of transferring the polarization can include the operations of mixing the compound and the target material, mechanically processing the mixture or dissolution of the compound or the target material. In various embodiments, the process of transferring the polarization can include the operations of dissolving the compound, mixing it with the target material and freezing the resulting mixture.

The operations of step 140 can be performed using one or more devices. In some embodiments, each operation can be performed by a different device, such as a pulverizing device, a mixing device, a polarization transfer device, a cross-polarization device, or a separation device.

The pulverizing device can include a cavity for pulverizing a compound, a pulverizer, and magnetic field generator. The pulverizing device can also include a port for introducing material (e.g., the compound or the target material) to the cavity. The pulverizing device can also include a cooler configured to cool the cavity during pulverization. The cooler can include a reservoir for holding liquid nitrogen or a cold gas cooling system.

A mixing device can include a cavity for holding the pulverized compound, and a mixing apparatus for mixing the pulverized compound into a mixture. The mixture can include the pulverized compound and a target material. The mixing device can also include a port for introducing the target material or a solvent for dissolving the target material. The mixing device can further include a cooler configured to cool the cavity during mixing. The cooler can include a reservoir for holding liquid nitrogen or a cold gas cooling system.

A polarization transfer device can include a magnetic field source and a cryostat, cold air flow or coolant, or chiller. In some embodiments, the polarization transfer device can include an NMR probe and spectrometer, enabling the detection and monitoring of the hyperpolarized signal during or after the polarization transfer.

A cross-polarization device can include a cavity for holding the mixture. In some embodiments, the cross-polarization device can include a magnetic field source and radiofrequency coils connected to a radiofrequency generator. The radiofrequency generator can be configured to produce two or more electromagnetic fields at two or more frequencies that excite nuclear spins in the mixture. In some embodiments, the cross-polarization device can be configured to translate the mixture through a magnetic field lower than 100 mT within a time of 10 seconds. For example, the time can be between less than 1 second in a magnetic field between 0.1-40 mT, transferring the polarization between the nuclear spins by low-field thermal mixing. For example, the cross-polarization device can be configured to include a conveyor that translates the first cavity through a location at the rate or within the time. The magnetic field source can be configured to maintain a magnetic field at the location during the conveying of the first cavity through the location.

A separation device can include a cavity for holding the mixture. The separation device can further include a magnetic field source and a port and a pump for introducing a solvent and extracting a dissolved product. The separation device can further include a particle filter (e.g., a sterile filtration membrane, or the like), through which flows the solvent with the dissolved target material, thereby removing contaminant particles of the compound. The separation device can further include a temperature control system.

In some embodiments, mixing and mechanical processing, as described herein, can be performed using a first device, while polarization transfer and cross polarization, as described herein, can be performed using another device. As a further example, the mechanical processing of step 140 can be performed together with the polarization of step 120, prior to transport. In such embodiments, the same device can be used to perform the polarization and the mechanical processing. Alternatively, the polarization of the compound can be performed in a separate device from the mechanical processing. In various embodiments, operations of step 140 can be performed by a single device. For example, the components and functionalities of the pulverizing device, mixing device, polarization transfer device, cross-polarization device, and separation device can be included in a single device.

In some embodiments, step 140 of process 100 can include a mixing operation. In the mixing operation, the target material can be exposed to the compound. As described above, the compound can be a solid, a glassy matrix, a powder, an aggregate, or in another suitable form. Similarly, in some embodiments, the target material can be a liquid (e.g., the target material can be dissolved in a solution), solid, a glassy matrix, a powder, an aggregate, or in another suitable form. In some embodiments, the target material can be crystalline.

In some embodiments, a liquid or amorphous material (e.g., a solvent) can be mixed with the compound and the target material to facilitate the transfer of polarization between the compound and the target material. For example, when the compound and the target material comprise solid microparticles (e.g., microcrystals), the liquid or amorphous material can fill interstitial spaces between the microcrystals. In this manner, the liquid or amorphous material can improve the contact between the microparticles. In some embodiments, the mixture can be cooled after addition of the solvent, thereby freezing or glassifying the mixture into a solid form.

The mixing operation of step 140 can be performed in a controlled environment. In some embodiments, the mixing can be performed in a temperature-controlled cavity. The mixing can be performed at a temperature less than 273 K, or preferably less than 253 K. The mixing can be performed in a non-zero magnetic field. In some embodiments, the magnetic field can be substantially spatially uniform throughout the controlled environment. For example, an amplitude inhomogeneity of the magnetic field throughout the controlled environment can be less than ±20%. In some embodiments, the magnetic field can be substantially temporally uniform throughout the controlled environment. For example, during the mixing a maximum or average amplitude inhomogeneity of the magnetic field throughout the controlled environment be less than ±20%. In some embodiments, an average strength of the magnetic field within the controlled environment during mixing can be at least 5 Gauss, more preferably at least 10 Gauss, more preferably at least 100 Gauss, or more preferably at least 1000 Gauss.

In some embodiments, step 140 of process 100 can include a mechanical processing operation. In the mechanical processing operation, the polarized compound can be mechanically altered to increase the surface area of the hyperpolarized compound. Increasing the surface area of the hyperpolarized compound can facilitate transfer of polarization from the hyperpolarized compound to the target material. In some embodiments, the target material and the mechanically altered polarized compound are in the form of microcrystals or nanocrystals. The mechanical processing can reduce the compound to micro or nano particles with a median size smaller than 1 mm³, or preferably smaller than 100 μm³. The mechanical processing can be performed by pulverizing the compound. For example, the compound can be disposed in a cavity, and a rod can be advanced into the cavity to crush the hyperpolarized compound. Alternatively, the rod can have a blending head, which can be spun to blend the hyperpolarized compound.

The mechanical processing of step 140 can be performed in a controlled environment. In some embodiments, the mechanical processing can be performed in a temperature-controlled cavity. The mechanical processing can be performed at a temperature less than 273 K, or preferably less than 253 K. The mechanical processing can be performed in a non-zero magnetic field. In some embodiments, the magnetic field can be substantially spatially uniform throughout the controlled environment. For example, an amplitude inhomogeneity of the magnetic field throughout the controlled environment can be less than ±20%. In some embodiments, the magnetic field can be substantially temporally uniform throughout the controlled environment. For example, during the mechanical processing a maximum or average amplitude inhomogeneity of the magnetic field throughout the controlled environment be less than ±20%. In some embodiments, an average strength of the magnetic field within the controlled environment during mechanical processing can be at least 5 Gauss, more preferably at least 10 Gauss, more preferably at least 100 Gauss, or more preferably at least 1000 Gauss.

In some embodiments, step 140 of process 100 can include a polarization transfer operation. In the polarization transfer operation, the nuclear spins of the mechanically altered polarized compound can be transferred to the nuclear spins of the target material. In some embodiments, the polarization transfer can occur via spin-diffusion by nuclei of the same species. The polarization transfer may additionally include transferring polarization within the target material from protons to nuclear spins having a lower gyromagnetic ratio than the gyromagnetic ratio of the protons.

In some embodiments, polarization transfer can be effectuated by applying multiple electromagnetic fields to the mixture. The electromagnetic fields can be applied at multiple frequencies that excite nuclear spins in the mixture. The polarization transfer can be performed in a controlled environment (e.g., a cavity of a device) and the two or more electromagnetic fields can have spatial or temporal amplitude inhomogeneities of at most ±20% within a predetermined portion of the controlled environment.

In various embodiments, polarization transfer can be effectuated by conveying the mixture through a controlled environment (e.g., a cavity of a device) containing a magnetic field at a minimum velocity or within a predetermined time period. In some embodiments, a strength of the magnetic field can less than 1000 Gauss, or preferably 600 Gauss, or more preferably 300 G, or less. In various embodiments, the predetermined time period can be 10 seconds, or preferably 1 second, or more preferably 0.5 seconds, or less.

In step 150, the target material can be separated from the compound. In some embodiments, the mixture of the target material and compound can be separated using a solvent. For example, when the target material and compound are mixed in a cavity, a solvent can be introduced into the cavity. In some embodiments, the solvent can be suitable for dissolving the target material but not the compound. The solvent can be mixed with the target material and compound, such that the target material is dissolved in the solvent. The solvent, containing the target material, can be withdrawn from the cavity, leaving substantially all of the compound. In some embodiments, the volume of the solvent can be larger than the volume of the mixture. In various embodiments, the solvent can be suitable for dissolving the compound but not the target material. Thus the solvent can be used to withdraw the compound from the cavity, leading substantially all of the target material.

Alternatively, in some embodiments, the target material can be exposed to the compound before polarization of the compound. For example, the target material can be mixed with the compound, as described herein, and then the resulting mixture can be polarized. In various embodiments, the mixture can include particles of the target material entrapped in polycrystals of the compound; particles of the target material entrapped in a single crystal or a mostly single crystal preparation of the compound; or the target material can be added to a powder of the micro- or nanoparticles of the PETS material.

Particles of the target material can be entrapped in single crystal(s) or polycrystals of the compound, consistent with disclosed embodiments. In some embodiments, particles of the target material can be overgrown by or encapsulated into the single crystal(s) or polycrystal(s). The particles of the target material can be micro- or nano-particles. In some embodiments, the target material can be introduced into a melt, solution, or vapor of the compound (or can have the compound grown around the particles of the target material by another crystal growth method).

In some embodiments, the target material can be added to a powder of particles of the compound, consistent with disclosed embodiments. In some embodiments, the compound can be present in the form of micro- or nanoparticles of one or more porous polycrystal(s).

In various embodiments, the target material can be a glassy solid and the micro- or nanoparticles of the compound can be entrapped in the glassy solid(s) of the target material. In some embodiments, the target material can be present in the form of one or more single crystal(s), mostly single crystal(s), or a polycrystal(s). In some embodiments, the target material can be a solution and the compound can be suspended in the solution.

Alternatively, in various embodiments, the target material can be exposed to the compound before the mechanical processing of the compound. For example, target material can be mixed with the compound (e.g., before or after hyperpolarization of the compound) and then the mixture can be mechanically processed.

A hyperpolarized biocompatible material can be manufactured, consistent with various embodiments in the present disclosure. The hyperpolarized biocompatible material can be manufactured by mixing the hyperpolarized biocompatible material with a non-biocompatible material containing nuclear spins into a mixture, wherein the non-biocompatible material includes a dopant with hyperpolarizable electron spins. The electron spins of the dopant can be hyperpolarized, and polarization can be transferred from the electron spins of the dopant to the nuclear spins of the non-biocompatible material. Moreover, polarization of the nuclear spins of the non-biocompatible material can be transferred to nuclear spins of the biocompatible material. In some embodiments, a second mixture containing the biocompatible material for injection into biological tissue can be prepared by at least in part, separating the second mixture from the first mixture, the second mixture including at least some of the biocompatible material and less than 1 mM of the non-biocompatible material. In some embodiments, the non-biocompatible material can be a molecular crystal.

In some embodiments, separating the second mixture from the first mixture can include differentially dissolving the biocompatible material and the non-biocompatible material into a solution using a solvent; and separating the solution from the mixture. In some embodiments, the solution is separated from the mixture using a filter. In some embodiments, the filter can have a pore size less than 200 nanometers.

In some embodiments, a polarity of the non-biocompatible material differs from a polarity of the biocompatible material; and separating the second mixture from the first mixture includes separating biocompatible material dissolved in the solution from non-biocompatible material dissolved in the solution using the difference in polarity. In some embodiments, the biocompatible material dissolved in the solution is separated from the non-biocompatible material dissolved in the solution using reversed-phase chromatography.

In some embodiments, separating the second mixture from the first mixture includes selecting biocompatible material to have a greater solubility in the solvent than the non-biocompatible material. In some embodiments, the solvent dissolves the non-biocompatible material and does not dissolve the biocompatible material.

In some embodiments, separating the second mixture from the first mixture includes dissolving the mixture in a combination of an organic solvent and an aqueous solvent, where the biocompatible material preferentially selected for dissolving in the aqueous solvent to form an aqueous solution and the non-biocompatible material preferentially selected for dissolving in the organic solvent to form an organic solution; and separation is performed by separating the aqueous solution from the organic solution.

In some embodiments, transferring polarization from the electron spins of the dopant to the nuclear spins of the non-biocompatible material comprises exposing the non-biocompatible material to a magnetic field.

In some other embodiments, a target material for NMR or MRI may be hyperpolarized by mixing the solvent containing the compound with a target material into a mixture. In some embodiments, at least 0.1 mg of a compound containing nuclear spins can be obtained, wherein the compound is hyperpolarized at a level of 0.1% polarization in a one Tesla magnetic field at room temperature. The compound can be dissolved in a solvent. In some embodiments, the solvent containing the compound is mixed with a target material into a mixture, and the mixture of the solvent and the target material is frozen within a predetermined time from the beginning of the mixing. In some embodiments, the compound is selected to have, under optical radiation, electron spins exceeding 1% polarization. In some embodiments, the compound contains a dopant which is selected to have, under optical radiation, electron spins exceeding 10% polarization.

In some embodiments, the mixture comprises a suspension of nanoparticles of the compound in a solution of the target material.

In some embodiments, the predetermined time is between 5 and 20 seconds. In some embodiments, the mixing of the solvent containing the compound and the target material includes co-dissolving the compound with the target material in a solution.

In some embodiments, obtaining the compound includes obtaining the compound, optically hyperpolarizing electron spins in the compound, and transferring polarization from the electron spins of the compound to nuclear spins of the compound, the transferring including exposing the compound to the magnetic field.

Compound Creation

As described above, with regards to FIG. 1, a compound having nuclear spins can be obtained. In some embodiments, the compound can serve as a polarization source for polarization transfer to a target material. In a preferred embodiment, the compound can be a PETS compound. Many organic molecules exhibit a phenomena that when excited with specific wavelengths in the optical or ultraviolet (UV) spectrum (1), electrons in a low level singlet state of the molecule S0 (2) get excited to a higher singlet electron state S1 (3), where either radiative decay back to the singlet state (4) or inter system crossing (ISC) to a triplet state (5) can occur. These triplet states exhibit two key features: First, they are long lived—on the order of microseconds to seconds—and can therefore be addressed on reasonable time scales. Second, the triplet state population between the three spin levels is non-uniform (6) for many molecules, thereby creating a polarized state.

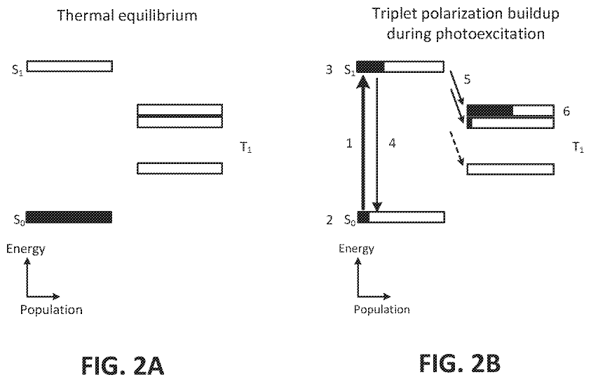

FIGS. 2A and 2B depict polarization of a triplet state population using photoexcitation. In each figure, the y axis denotes the energy of the different spin levels, and the filled bars denote the population of each spin level. As depicted in FIG. 2A, while in thermal equilibrium all of the electron population is in the ground singlet state. As depicted in FIG. 2B, during photoexcitation the triplet state becomes populated in a non-uniform fashion, with one spin state becoming more populated than the other two triplet states.

Several such molecules, e.g. acridine, pentacene, benzophenone, can have one of the spin states over 90% populated, thereby creating almost unity polarization at temperatures and magnetic fields where the thermodynamic polarization of the electron spins is orders of magnitude smaller. Moreover, the different triplet spin states (5) can exhibit different decay times to the singlet state, thereby creating another process where a differential population between the spin states, and therefore polarization, can be obtained.

An important advantage of these optically excited triplet states is that the electrons decay from the triplet state back to the singlet ground state. Free electrons can be a principal source of nuclear relaxation at lower temperatures. Thus, when the electrons in a molecule decay back into the singlet state (and are therefore without free electron spin), the molecule no longer contains paramagnetic impurities due free electron spins which can relax the surrounding nuclear spin. Therefore, the nuclear spin polarization can reach a higher level, and a material can have a significantly longer relaxation time following the polarization sequence.

In a preferred embodiment, the polarization molecules can be incorporated into a compound. The proportion of the compound that comprises polarization molecules can vary. Polarization molecules can be added as dopants into a compound composed mostly of other types of molecules, or they can be used in a compound which consists of a substantial amount of polarization molecules.

The compound can be produced in several different ways. In some embodiments, the compound can be produced in a form of crystal grown from a melt. The compound crystal can be grown from the melt in several crystal growth methods, including rapid temperature reduction, the Bridgman growth method, Czochralski method, the cell method, or other known crystal growth methods. In a preferential embodiment the polarization molecules are included in the melt in the desired concentration.

In various embodiments, other crystal growth methods that are known in the literature can be used, including crystal growth from a solution, gel or vapor. Several growth methods are detailed in “Growth of bulk single crystals of organic materials for nonlinear optical devices: an overview” by Penn, Benjamin G., et al. The relevant portions of this publication with regard to molecular crystal growth and purification are incorporated into the present disclosure by way of reference, including growth by physical vapor transport, growth from the melt via the Bridgman-Stockbarger Method, Czochralski Growth or Kyropoulos Method, growth from solutions including slow cooling processes, solvent evaporation processes and temperature difference processes.

In another embodiment, the polarization molecules can be added to a Shpolsky matrix. Pentacene for example can be incorporated into several Shpolsky matrices, including n-heptane, n-nonane, n-decane, n-dodecane, n-tetradecane and n-hexadecane. A method for such incorporation is disclosed in “Spectroscopic characteristics of pentacene in Shpol'skii matrixes”, by Banasiewicz, M., I. Deperasińska, and B. Kozankiewicz and incorporated herein by reference. As described in this paper, liquid samples can be bubbled with argon to remove oxygen and gently heated to increase the host solubility. Liquid samples can then be quickly frozen in liquid nitrogen before being inserted into the polarizer cryostat.

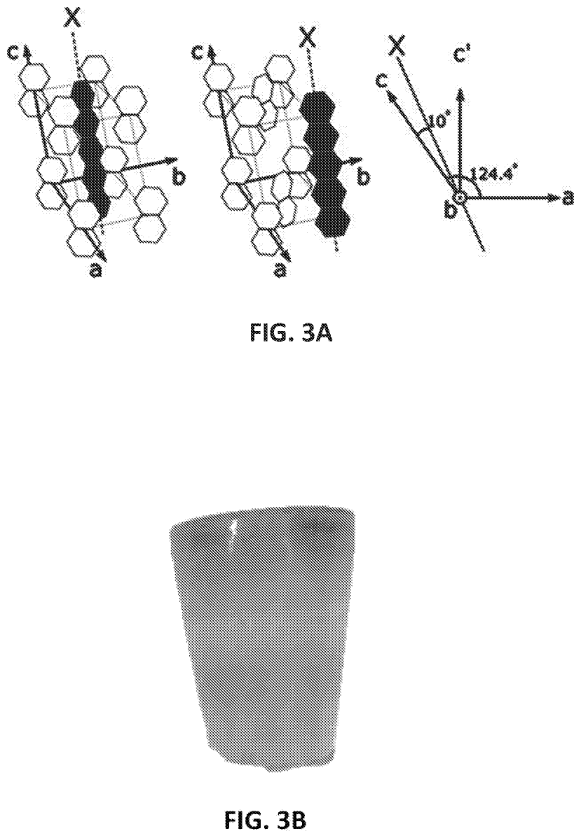

In some embodiments, the compound can be a pentacene:naphthalene crystal. As depicted in FIG. 3A, pentacene dopants can be incorporated into a crystal lattice of the naphthalene crystals in two possible orientations. The presence of such defined orientations can enable hyperpolarization, consistent with disclosed embodiments. Relatively high amounts, up to around 10\hat{ }(−4) mol/mol, of pentacene can be doped into a naphthalene crystal. An example of such a pentacene:naphthalene crystal is shown in FIG. 3B.

In some embodiments, the self-seeding vertical Bridgman technique can be used to grow a pentacene doped naphthalene single crystal. In a variant of Bridgman growth, a double walled ampule can be used, where the inner wall has an open capillary towards the interspace between the walls. The ampule can be filled with naphthalene and pentacene and then moved through a steep temperature gradient, which includes the melting temperature of naphthalene. This temperature gradient can be achieved by a bath with two liquid phases, which are heated to different temperatures. When the ampule is lowered into the upper and warmer part of the bath, the pentacene-naphthalene mixture melts into a homogeneous liquid. Once the bottom of the ampule reaches the phase separation in the heating bath, crystallization starts in the interspace between the ampule walls. Here, the solidification happens with multiple nuclei, leading to a polycrystalline area in the interspace between the walls. By moving the ampule slowly within that region, the number of nucleation events can be kept minimal, leading to a polycrystal with relatively large grains. Once the ampule is lowered further, the capillary of the inner wall gets in contact with the polycrystal. Ideally, the crystal orientation of only one single grain forms within the capillary. That self-seeding process favors the emergence of a single crystal within the inner wall of the ampule.

Compound Polarization

As described above, with regards to FIG. 1, the nuclear spins in the compound can be polarized. In some embodiments, the polarization can be accomplished by exposing the compound to extreme temperatures and magnetic fields. As a non-limiting example, DNP can be performed at low temperatures and high magnetic fields. At temperatures below 4K and magnetic fields above 1 T, the free electron spins in radicals or paramagnetic defects in the material are highly polarized in thermodynamic equilibrium. Using DNP protocols, this high thermal polarization can be transferred to nuclear spins in the compound. In some embodiments, when the compound comprises a solution of polarization molecules, dissolution DNP can be used for transferring nuclear spins. As an additional example, brute force polarization can be used to polarize nuclear spins in the obtained compound. In some embodiments, the compound can be placed at below 1 K temperatures and greater than 5 T magnetic fields, where the nuclear spins in the compound are highly polarized. In some embodiments, polarization is transferred from nuclear spins with a high gyromagnetic ratio (e.g., protons) to nuclei with a lower gyromagnetic ratio.

In some embodiments, the polarization can be accomplished optically. For example, optical polarization can occur using optical defects such as color centers. In such methods, optically active defects in semiconductors such as diamond and silicon carbide can be used to polarize surrounding nuclear spins. As an additional example, optical polarization can hyperpolarize the nuclear spins in a PETS compound by polarization transfer from optically polarizable electron spins in the PETS polarization molecules to the nuclear spins.

Transfer of Polarization from Electron Spins to Nuclear Spins

Polarization can be transferred from electron spins to nuclear spins using methods including dynamic nuclear polarization (DNP). DNP methods can use microwave or radio frequency irradiation or magnetic field tuning to transfer electron spins to nuclear spins. Such methods can lead to polarization transfer through level avoided crossing (LAC), or other suitable phenomena. DNP protocols can exploit at least one of interactions between electron spins or underlying physical mechanisms (e.g., fulfilling a resonance condition, such as the Hartmann-Hahn condition, or excitation of selective transitions, such as irradiation at a frequency matching the energy gap between two quantum states). DNP protocols can differ in the configurations used to achieve these conditions. DNP protocols can also differ in the usage of microwave pulses or continuous microwave radiation. Examples of DNP methods are disclosed herein with regards to PETS compounds, as high nuclear polarizations can be obtained in PETS compounds using DNP methods (e.g., >10%, >50% or >80%). However, the disclosed embodiments are not limited to PETS compounds.

Suitable DNP methods consistent with disclosed embodiments are discussed in “Room temperature hyperpolarization of nuclear spins in bulk”, by Tateishi, Kenichiro, et al. (e.g., for pentacene:p-terphenyl), “High proton spin polarization with DNP using the triplet state of pentacene-d14”, by Eichhorn, T R, et al. (e.g., for pentacene:naphthalene), “Dynamic nuclear polarisation by photoexcited-triplet electron spins in polycrystalline samples”, by Takeda, Kazuyuki, K. Takegoshi, and Takehiko Terao (e.g., polycrystalline samples of pentacene:naphthalene with random crystal orientations). Suitable DNP methods are also disclosed in Section II of “Dynamic nuclear polarisation at high magnetic fields”, by Maly, Thorsten, et al. In addition, sophisticated DNP sequences such as those disclosed “Robust optical polarization of nuclear spin baths using Hamiltonian engineering of nitrogen-vacancy center quantum dynamics”, by Schwartz, Ilai, et al, can enable fast polarization transfer. Suitable DNP methods disclosed in “Dynamical nuclear polarization using multi-colour control of color centers in diamond”, by Yang, Pengcheng, Martin B Plenio, and Jianming Cai, and “Enhanced dynamic nuclear polarization via swept microwave frequency combs” by Ajoy, A, et al. can enable nuclear polarization transfer in nanocrystals, or polycrystalline source materials or bulk samples (e.g., using colour centers in nanodiamonds). The DNP methods and preparation techniques disclosed in these references are incorporated herein by reference.

In some embodiments, a polarization sequence can include a polarization step followed by a transfer step. In the polarization step, the compound can be exposed to a strong optical pulse. The duration of the optical pulse can be 100 ns to 10 μs. The energy in the optical pulse can be between 0.1 mJ and 10 mJ, or greater. The energy and duration of the optical pulse can be selected to populate triplet states of polarization molecules in the compound in a polarized fashion.

In some embodiments, electron spins can be transferred to hydrogen nuclear spins in the compound in the transfer step using the integrated solid effect (ISE). By changing the parameters of the transfer step, other species of nuclear spins may be affected. In some embodiments, for example, electron spins can be transferred to ¹³C nuclear spins in the compound by using a different B1 microwave (MW) field. In various embodiments, electron spins can be transferred to nuclear spins in the transfer step using alternatives to ISE. For example, the solid effect, the cross effect, or low-field thermal mixing (in the case of a very high concentration of the PETS molecules) can be used to effect spin transfer. As an additional example, pulsed DNP methods such as the NOVEL sequence or dressed-state solid effect can be used to effect spin transfer.

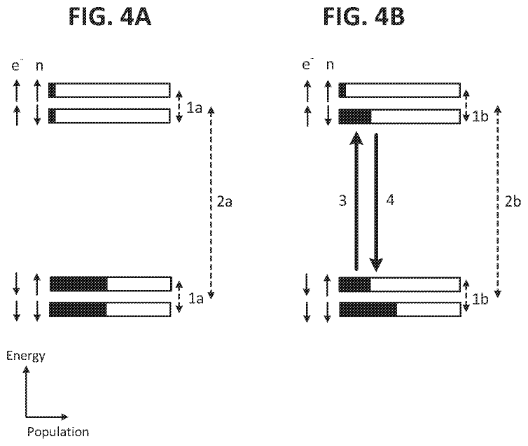

FIGS. 4A and 4B depict spin transference that occurs during an exemplary DNP method that achieves spin transfer using the Solid Effect. This exemplary method transfers polarization from the electron spin to the nuclear spin, increasing the nuclear polarization while decreasing the electron polarization. FIGS. 4A and 4B depict the different electron/nuclear spin states in a compound as four levels, with the black bar representing the population in the compound at each level. Prior to initiation of spin transfer, as shown in FIG. 4A, the compound exhibits greater electron spin polarization than nuclear spin polarization. The two bottom levels therefore are depicted with a greater population than the two top levels. Using microwave or rf irradiation on resonance with the so-called forbidden transition between the states |↑>ₑ|↓>ₙ↔|↓>ₑ|↑>ₙ saturates the population of the two states |↑>ₑ|↓>ₙ, |↓>ₑ|↑>ₙ. This saturation increases the overall population of the |↓>ₙ state and reduces the overall population of the |↓>ₑ state. Therefore the nuclear polarization is increased while the electron polarization is decreased, as shown in FIG. 4B, effectively transferring polarization from electron spin to the nuclear spin.

In some embodiments, the electron spins can be transferred to the nuclear spins using an interaction involving at least two electron spins and a nuclear spin (e.g., using cross effect and low-field thermal mixing DNP protocols). Such an interaction can rely on allowed transitions of several electron spins and a nuclear spin involving a homogeneously or inhomogeneously broadened electron paramagnetic resonance (EPR) line. Energy can be conserved in the broadening of the EPR line when two or more electron spins and a nuclear spin are flipped simultaneously.

In various embodiments, the electron spins can be transferred to the nuclear spins using a variant of ISE in which a multi-frequency microwave “comb” sweeps several microwave frequencies in parallel. Such a technique can be particularly suitable for transferring polarization in nanocrystals, polycrystalline source materials or bulk samples.

In some embodiments, triplet lifetime can be extended and the polarization of the compound increased by preparing the triplet state before the DNP protocol. This can be done via a population transfer between the excited state sublevels (e.g. by a 180-degree pulse resonant with the transition frequency, or the like) to a different spin state with a longer relaxation time. Additional details of preparing a triplet state before a DNP protocol are provided in “Dynamic Nuclear polarisation with Paramagnetic Centers Created by Photo-Excitation”, by Eichhorn, Tim Rolf, and incorporated herein by reference.

In some embodiments, polarization transfer from the electron spins to the nuclear spins can be achieved without using microwaves by tuning an external magnetic field to the level avoided crossing (LAC) of the electron spins. Additional details of polarization transfer are provided in “Dynamic Nuclear polarization with Paramagnetic Centers Created by Photo-Excitation”, by Eichhorn, Tim Rolf, and incorporated herein by reference.

In some embodiments, the external magnetic field can be selected according to the desired application. For example, for polarization of target molecules for hyperpolarized MRI applications or NMR spectroscopy in an external spectrometer, the magnetic field is preferably smaller than 4 T. The method according to the invention allows for the use of external magnetic fields with a low magnetic flux density, preferably below 2 T, more preferably below 1 T, for example below 0.5 T, for example below 0.05 T. Advantageously, many of these magnetic flux densities can be achieved by a permanent magnet or an electromagnet, which does not rely on superconducting material at very low temperatures. Magnetic field can be measured via conventional methods such as with a gaussmeter.

Advantageously, the induced relaxation of the nuclear spins in the compound can be reduced by means of actively decoupling the nuclear spins from possible electron spins on the surface of the compound due to contaminants. This can be achieved by driving the electron spins with microwave or radio frequency irradiation at their Larmor frequency or energy transition frequencies (in the case there is a strong hyperfine splitting or spin 1 electron spin), or in the electron-nuclear zero-quantum or double-quantum resonance conditions.

In some embodiments (e.g., NMR spectroscopy applications), it can be advantageous to perform the polarization transfer from the optically polarizable electron spins to the nuclear spins in-situ (e.g., in the NMR device). The same magnet can then be used for polarization transfer and for performance of the NMR spectroscopy. For compounds including polycrystals, single crystals, or single crystals in the form of micro or nanoparticles, a low magnetic field below 50 mT can enable addressing many of the orientations of the PETS electron spins.

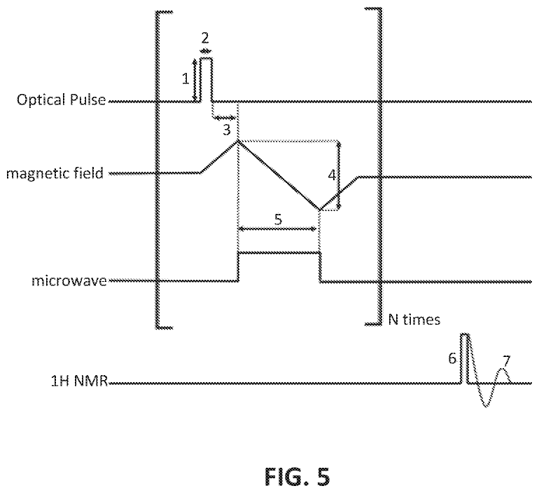

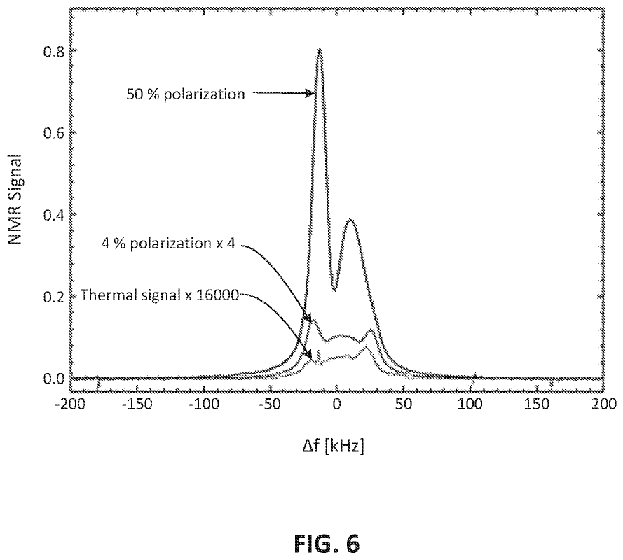

FIG. 5 depicts an exemplary sequence of optical irradiation, magnetic field sweep and electromagnetic irradiation (e.g., a polarization sequence) suitable for inducing polarization in a compound. Such a sequence can include at least one of optical irradiation, magnetic field sweep or electromagnetic irradiation. In some embodiments, the compound is a pentacene-d14:naphthalene-h8 crystal sample. The sample can be as large as 100 to 300 mm\hat{ }3. In some embodiments, the sample can be cooled to 100 K or lower, while placed in a magnetic field of 1 to 3 kG that is oriented along the pentacene molecules' long axis. The sequence can include multiple repeats of an optical pulse followed by a magnetic field sweep. In each repeat, one or more optical pulses (e.g., laser pulses) can excite the pentacene molecules into a short-lived triplet state. This can be achieved by populating a higher singlet state with optical pulses of few to several 10 mJ pulse energy (1) in a time window (2) of up to a few microseconds in which the slower interstem crossing from the singlet to the triplet state takes place. After a short delay (3) of few 100 ns, the magnetic field, which has been ramped up before, sweeps through the full triplet's electron spin resonance linewidth (4) of a few G, while constantly irradiating with microwaves (5) in order to facilitate Hartmann-Hahn matching of all spin packets within the line. After repeating this sequence for N times, the proton signal can be read out via the free induction decay (7) of a resonant radiowave pulse with a non-destructive small tip-angle amplitude (6). As depicted in FIG. 6, this sequence of optical and magnetic interactions can increase polarization in the sample to greater than 50%.

FIG. 6 depicts NMR signal reads from a compound before and after repeated iterations of the polarization sequence depicted in FIG. 5. In FIG. 5, the depicted X-axis is the frequency of the NMR signal and the depicted y-axis is the signal strength. A first trace depicts the NMR signal from the compound at thermal equilibrium (multiplied by 16,000). A second trace depicts the NMR signal read from the compound with 4% polarization (multiplied by 4). A third trace depicts the NMR signal read from a compound with 50% polarization. The increase in polarization can be the result of repeated iterations of the polarization sequence depicted in FIG. 5.

Compound Transport

As described above with regards to FIG. 1, after the compound is polarized, the compound can be transported to a destination location from an origin location. Consistent with disclosed embodiments, the compound can have a long nuclear relaxation time. Accordingly, the compound can be stored and transported without undergoing an unacceptable degree of depolarization (e.g., above 90% depolarization). Because the polarization of the compound can be performed separately from any further processing of the compound, polarization and further processing can be performed by separate devices, each optimized for different purposes. Furthermore, production of the polarized compound for sufficient multiple end-users can be performed at a centralized facility, enabling greater efficiencies and economies of scale.

In some embodiments, the polarized compound can be transported to the destination location and then processed into micro- or nanoparticles, prior to transferring of polarization to a target material. In some embodiments, the polarized compounds can be processed into micro- or nanoparticles prior to transportation to the destination location.

A transportation device can be configured to transport samples of the compound. The transportation device can be arranged and configured for transporting one or more samples simultaneously. The transportation device can be configured to maintain the one or more samples in a magnetic field of at least 10 G, more preferably 100 G, more preferably 1000 G.

A permanent magnet or an electromagnet included in the transportation device can provide the magnetic field. Moreover, in some embodiments, the permanent magnet or electromagnet can be shielded to reduce the strength of the magnetic field outside the transportation device. The transportation device can also include a cooling system. The cooling system can be configured to maintain samples at a predetermined temperate or within a predetermined range of temperatures during transport. For example, the cooling system can be configured to maintain the samples at a temperature below 270K, below 80K, or below 4K. In some embodiments, the transportation device can be configured to maintain the samples at approximately the temperature of liquid nitrogen. The transportation device can include insulation between the cooling system and the exterior of the transportation device, to minimize heat exchange with external environment. In some embodiments, the cooling system can be configured to maintain the temperature of the samples using a cold gas flow. In various embodiments, the cooling system can be configured to maintain the temperature of the samples using a liquid coolant. In various embodiments, the transportation device can include a Dewar to provide cooling of the samples. In order to distribute the polarized samples also across large distances, the container preferably can be transported by standard transportation vehicles, such as planes, trains, trucks, cars and ships.

Polarization Transfer to Target Materials

As described above with regards to FIGS. 2A to 6, a compound can be created and polarized. In some embodiments, the compound may then be transported to a destination location. The polarization of the compound can then be transferred to a target material. In some embodiments, the transfer of polarization can include preparatory steps of increasing the surface area of the compound and mixing the compound with the target material.

The transfer of polarization from the nuclear spins of the compound spins to the nuclear spins of the target material or a mediator can occur at the surface of the compound. The efficiency of polarization transfer can be dependent on the surface area, with larger surface area resulting in improved polarization transfer. Thus the surface area of the compound can be increased to increase the efficiency of polarization transfer to the target material.

In some embodiments, the surface area of a solid compound can be increased by pulverizing the solid compound. Pulverizing the compound can include reducing the median particle size in a sample of the compound. Pulverizing can include crushing, squashing, grinding, squeezing, pressing, milling or breaking down the sample of compound. The disclosed embodiments are not limited to a particular method of pulverizing the compound. The sample quantity pulverized can be between 1 ng and 1 g, or greater. In some embodiments, the pulverized compound can include smaller micro- or nanoparticles. The micro- or nanoparticles can include single crystals, mostly single crystals, or polycrystals. The median size of the pulverized compound can be between 1 cubic millimeter and 1 cubic micrometer, or smaller.

In some embodiments, these preparatory steps can be performed before or after transportation of the polarized compound. In various embodiments, these preparatory steps can be performed before polarization of the compound. Increasing the surface area of the compound after transport can be more efficient than increasing the surface area of the compound before transport. Nuclear relaxation times are typically longer and polarization build-up more efficient when the compound is in bulk form. Thus polarization can be more efficiently stored and transported when the compound is in bulk form. However, polarization transfer to surrounding molecules can be more efficiently performed after increasing the surface area of the compound (e.g., polarization can be more effectively performed in a micro- or nano-particle or molecule composition, where the surface area is very large). In some embodiments, the compound can then be mixed with a target material. At least some of the polarization of the compound can be transferred to at least some of the target material. Mixing the compound with the target material can include, or be preceded by, operations to increase the surface area of the compound. These operations can be performed while preserving the polarization of the compound.

Pulverization Conditions

The surface area of the sample of the compound can be increased under conditions that preserve at least some of the polarization of the sample. Consistent with disclosed embodiments, between 10% and 70%, or more, of the original polarization of the sample can be retained while increasing the surface area of the sample.

In some embodiments, the sample can be maintained in magnetic field having a minimum field strength during pulverization (e.g., a field strength between 10 Gauss and 1000 G, or larger). In various embodiments, the sample can be maintained at a temperature selected based on the temperature dependence of the materials' nuclear relaxation time. For example, the nuclear relaxation time of urea and naphthalene monotonously increases with decreasing temperature, while the nuclear relaxation time of p-terphenyl and pyruvic acid can decrease when with decreasing temperature. In some embodiments, the sample can be maintained at a temperature lower than room temperature, preferably below minus 20° C., more below minus 50° C., more preferably below minus 100° C. or at or below the temperature of liquid nitrogen in order to prolong the nuclear relaxation time. In some embodiments, the sample can be maintained in an inert atmosphere to prolong its nuclear relaxation time. For example, surface reactions can occur between naphthalene and oxygen. Therefore, in a preferred embodiment the sample is preferably kept in a nitrogen, argon or vacuum atmosphere while the surface area of the sample is being increased, for example, by pulverizing.

Mechanisms of Pulverization

In various embodiments, the pulverizing of the compound can be performed using a mechanical device for the grinding or pulverizing of the compound, friction-based pulverization (e.g., using a mortar and pestle or the like), mechanical milling (e.g., ball milling, plenary milling, rod milling or vibratory milling, or the like), cryo-milling, ultrasound cavitation or machining (e.g., using a high pressure cell or the like), and other methods. The disclosed embodiments are not limited to a particular pulverization method.

In some embodiments, pulverization can be performed at a predetermined temperature or temperature range. The predetermined temperature or temperature range can be selected based on a temperature dependence of relevant characteristics of the compound (e.g., friability, hardness, or the like). In some instances, performing pulverization at a temperature or temperature range in which the compound is friable can increase the efficiency of pulverization. For example, a compound including soft crystals can become more brittle at lower temperatures. Pulverizing the compound at such a temperature can make the pulverization into nano- or microparticles more efficient.

Mixing of Compound with Target Material

The polarized compound can be mixed with the target material. The mixing can occur prior to transfer of polarization. The mixing can be performed to increase the contact area between the polarized compound and the target material, without causing the polarized compound to become depolarized. The mixing can be performed using a variety of methods, as described herein. The mixing can be performed with the polarized compound in a solid, liquid, or gas form and the target material in a solid or liquid form, consistent with disclosed embodiments. For example, the mixing can be performed using a solid compound and liquid target material, a solid compound and solid target material, or a liquid or gas compound and liquid or solid target material. In some embodiments, a mediator or additional compound can be added to the mixture to improve the efficiency of polarization transfer.

Solid Compound and Liquid Target Material

A target material in a liquid form (e.g., a liquid phase of the target material or a solution of the target material) can be brought into contact with a solid pulverized compound, consistent with disclosed embodiments. The solid pulverized compound can include hyperpolarized micro- or nanoparticles and the liquid target material can be mixed with, or placed on, the solid pulverized compound.

In some embodiments, the pulverized compound can be compressed before or after contacting the pulverized compound with the target material. Such compression can reduce the distances between particles of the pulverized compound (e.g., reducing voids). By reducing distances between particles, such compression can improve transfer of polarization between the compound and the target material.

In some embodiments, the solution containing the target material molecules are composed of biocompatible matrices, including water, water/glycerol and DMSO mixtures.

In some embodiments, the pulverized compound can have a dense, porous structure through which liquid target material can be introduced. The liquid target material can subsequently be solidified. For example, the liquid target material can subsequently be solidified (e.g., in to a crystalline or amorphous solid). In some embodiments, the solid pulverized compound can be suspended in a solution of liquid target material.

In some embodiments, a mixture containing the target material can be cooled to form a polycrystal or glass hosting the pulverized compound. For example, a suspension of the pulverized compound in a solution of liquid target material can be solidified by reducing the temperature.

In some embodiments, the pulverized compound can be maintained during mixing at a temperature (or within a temperature range) and magnetic field (or within a magnetic field strength range) at which pulverized compound has a long Ti relaxation time. In some embodiments, the mixture can be cooled at a rate that permits sufficient mixing between the pulverized compound and the liquid target material before solidification of the liquid target material. In some embodiments, the rate can be controlled. For example, the temperature of the mixture can be maintained on a predetermined trajectory. In various embodiments, the rate can arise from the design of the cooling system. For example, the mixture can be maintained in a first temperature in an environment with cooling (e.g., cooling by cold nitrogen gas). After addition of the liquid target material, the mixture may be at a second temperature. The mixture may be cooled to a third temperature using the cooling system. The rate of cooling may be sufficient for the liquid target material to encapsulate the particles of the pulverized compound before solidifying. In some embodiments, the target material can be co-located with the compound while the compound is being pulverized. In some embodiments, the target material is in a solid form (e.g., a glass or crystalline form). In such embodiments, the mixture can be heated following pulverization to cause the target material to dissolve. The mixture can then be cooled to create a solid hosting the particles of the pulverized compound.

Polarization Example—Pentacene:Naphthalene

A compound including a pentacene:naphthalene crystal was polarized to >40% 1H nuclear polarization via PETS, as described herein. The polarized crystal was then placed in a sample holder of a transport device. The transport device included permanent magnets disposed on each side of the holder to maintain the sample in a magnetic field. The sample holder was placed into a Styrofoam box filled with liquid nitrogen.

The polarized crystal was transferred to a pulverization apparatus. The pulverization apparatus included a 5 mm NMR tube for holding the sample, an 0.5 T magnetic field supplied by a permanent magnet a home-built NMR probe integrated with a Kea2 spectrometer for measuring and monitoring the nuclear polarization and a motorized glass rod chosen to fit precisely into the NMR tube for pulverizing the polarized sample.

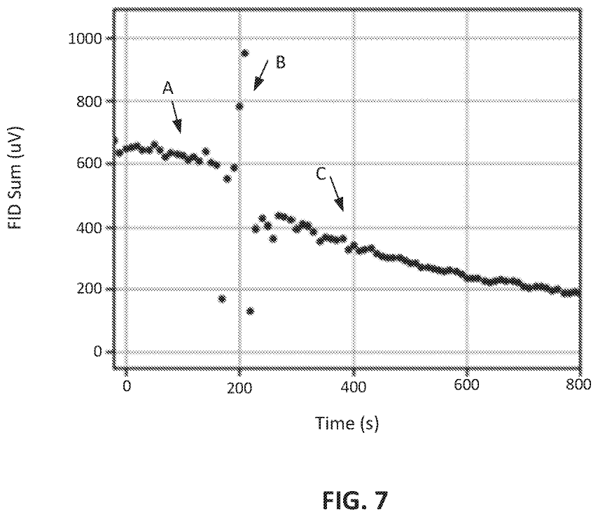

FIG. 7 depicts an exemplary decrease in polarization of a compound over time. An NMR signal (FID sum—integration of the signal from the free induction decay of the nuclear spins) indicates the degree of polarization of the compound and is acquired from the compound over time using 0.80 flip angle (0.5 us pulse, −35 dB power). In the timeframe labeled “A”, the signal from the polarized crystal showed little decay in the polarization (e.g., the magnitude of FID sum) due to the long relaxation time of the crystal. In the timeframe labeled “B” the crystal was crushed to microparticles, with the NMR signal showing large deviations due to the large motion and vibration of the sample and sample holder. In the timeframe labeled “C” the NMR signal was acquired from the polarized pulverized powder. The transition between the timeframes labeled “A” and “C” shows little loss of polarization, and most of the loss can be attributed to the loss of some material due to a fraction of the powder remaining on the NMR tube following the pulverization.

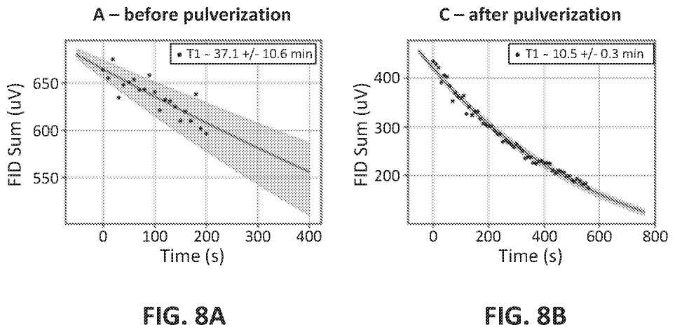

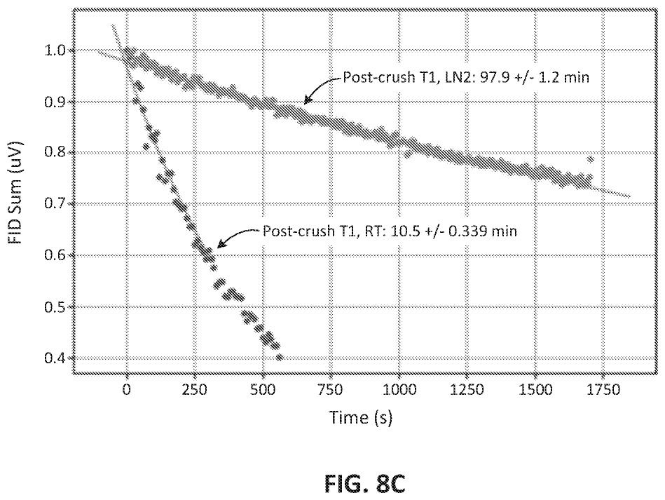

In some embodiments, the compound can retain a long relaxation time following pulverization. This long relaxation time can enable mixing the compound with the target material and transferring of polarization to the target material. FIGS. 8A and 8B depict exemplary polarization time dependence for timeframes “A” (before pulverization) and “C” (after pulverization). It can be seen that while the relaxation time decreases, it is still on the order of 10 minutes even at room temperature. FIG. 8C depicts the possible enhancement in the relaxation time of the pulverized microparticles achievable by lowering the temperature to 77K using a liquid nitrogen Dewar when measuring the hyperpolarized 1H nuclear spins in the pulverized naphthalene microparticles at 0.5 T magnetic field.

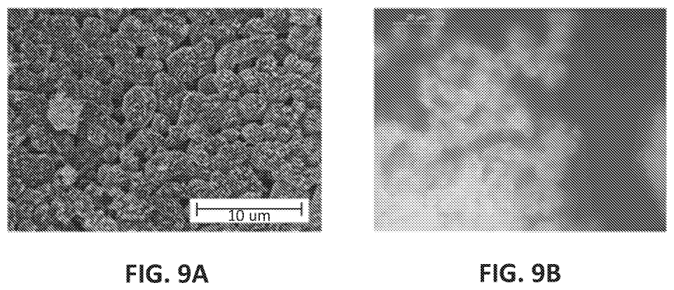

FIGS. 9A and 9B depict exemplary scanning electron microscope (SEM) and optical microscope images of a pulverized naphthalene sample. A fairly uniform size distribution is achieved, with a median size significantly below 10³ μm³.

Similar results can be obtained by mixing the pulverized pentacene:naphthalene compound in an aqueous solvent which does not dissolve the naphthalene microparticles (e.g., water, D2O, water/ethanol, water/glycerol and water/pyruvate mixtures). After introducing the solvent and the target materials, the pulverization apparatus can be used to thoroughly mix the powder and solvent and produce a homogenous mixture. For the water/ethanol and water glycerol solvents, the mixture can be kept in a magnetic field and lowered into a liquid nitrogen Dewar beyond the glass temperature of the solvents, creating a mixture of polarized naphthalene microcrystal in a glassy matrix.

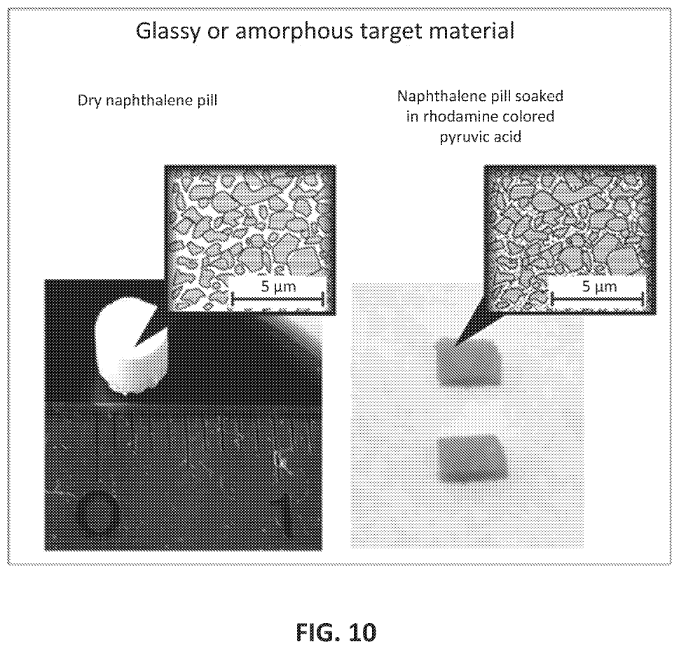

As depicted in FIG. 10, the polarized naphthalene microcrystals can be mixed with the target material in a solution, consistent with disclosed embodiments. In some embodiments, a pulverized naphthalene powder can be pressed into a pill with mechanical pressure, reducing the pore sizes between the naphthalene particles. This could be measured by changes in the weight to volume ratio of the naphthalene pill. Following the formation of the naphthalene pill, liquid pyruvic acid mixed with trace amounts of rhodamine, the target material in this embodiment, can be injected on top of the pill. The liquid pyruvic acid can quickly (in several tens of seconds) soak into the pill, wetting the naphthalene microparticles. This can be observed from the rhodamine coloring of the pyruvic acid solution. Lowering the soaked pill into a liquid nitrogen Dewar produces a densely packed pill composed of naphthalene microparticles with the target material in a glassy state wetting the particles and filling the inter-particle voids (as depicted in the inserts in FIG. 10).

In certain embodiments surface molecules of the compound microparticles can undergo proton exchange with the surrounding solvent or with the target material molecules. This enables polarized protons from the compound to exchange to the target molecules and in this way a polarization transfer is achieved. As proton exchange can occur on similar timescales as proton-proton spin diffusion in a solid, the compound microparticles can serve as a continuous source for polarization for the exchanging proton spins. In certain embodiments, 1H nuclear spins with exchangeable protons are added to the surface of the compound microparticles by a chemical reaction, introducing for example OH or NH2 groups to the surface of the microparticles. In other embodiments, the surfaces of the compound microparticles are coated with a coating molecule which can exchange protons with the solvent or target molecules. This coating can be achieved for example by adsorption of the coating molecules to the compound microparticles. For example, if the compound molecules are nonpolar and the solvent is polar, certain non-polar molecules could preferentially adsorb on the compound surface.

Solid Compound and Solid Target Material

A solid compound can be mixed with a solid target material, consistent with disclosed embodiments. The solid target material can include micro- or nano-crystals. The solid target material can be an amorphous solid. As described above, the solid target material can be co-pulverized with the compound. Additionally or alternatively, a solid target material can be mixed into the pulverized compound in powdered form. In certain embodiments, in order to improve the contact between the target material and compound, pressure can be applied to the solid target material and compound during pulverization or following the mixing of powdered (or pulverized) target material and pulverized compound. Advantageously, such compression can be used for bringing the compound and target material in contact without a heating step or with heating to a lower temperature than required for mixing with the target in liquid form.

A solid compound can be deformed to increase the contact area with a solid target material without forming a powder. As a non-limiting example, pentacene:naphthalene is soft when broken down at room temperature but can still be put into high contact with particles of a solid target material, especially when mixed together.

Liquid Mediator for Solid-Solid Mixtures

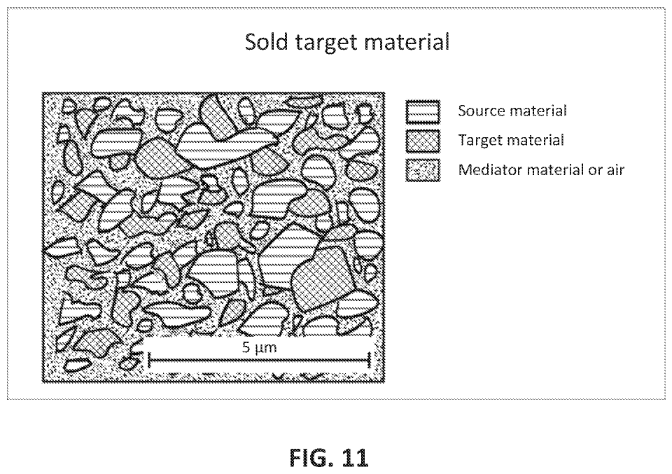

As depicted in FIG. 11, an amorphous or liquid mediator can be added to a mixture of powdered (or pulverized) target material and pulverized compound, consistent with disclosed embodiments. The mediator can fill voids between particles in the mixture, thereby establishing contact between particles of target material and compound and increasing the efficiency of polarization transfer. In some embodiments, the mediator can be a liquid that wets but does not significantly dissolve both the source and target nanoparticles. The mediator can be added to the mixture as liquid at a first temperature, then cooled from a second temperature to a third temperature. During cooling, the mediator can freeze into a glassy state. The mediator can be selected based on the polarity and chemical composition of the compound and target material. In some embodiments, the mediator can be or include common glassifying agents and solvents such as water/glycerol mixtures, water/dmso mixtures, toluene-based solvents, or other suitable glassifying agents and solvents. Such glassifying agents and solvents are still liquid at temperatures below 0° C. Furthermore, as dissolution concentrations of molecules in these solvents is very temperature dependent, below 0° C. most molecules will not dissolve in a high concentration, thereby enabling the solvent to be used as a mediator if introduced to the mixture at that temperature range.

Liquid or Gas Compound and Liquid or Solid Target Material

In some embodiments, a solution can be produced by dissolving a polarized compound in a solvent. The compound can be soluble in the solvent and can be selected to retain polarization after dissolution. For example, compounds such as naphthalene have a long relaxation time when dissolved at room temperature or other temperatures in the range of minus 150 C to 100 C, enabling the dissolution while preserving the polarization of the source molecules. Potential solvents can depend on the selected compound. As a non-limiting example, when the polarized compound is naphthalene, the potential solvents can include toluene, ether, ethanol, carboxylic acids, chloroform, hexane, acetic acid, butyric acid and mixtures or derivatives thereof.

In some embodiments, both the polarized compound and the target material can be solutes in the solution. As a non-limiting example, the target material can be dissolved into a solution of the polarized compound and solvent. Alternatively, the polarized compound can be dissolved into a solution of the target material and solvent.

In some embodiments, the target material can be suspended in the solution of the polarized compound and solvent. For example, the target material can be mixed into the solution in nano/micro-crystal, polycrystal or amorphous form.

In some embodiments, the solvent and target material can be chosen to enable the exchange of protons between the polarized compound and at least one of the solvent and the target material. Such a proton exchange can facilitate the polarization transfer to the target material as the polarized protons of the compound can be exchanged with protons in the target material. Polarization from the exchanged protons may then transfer to other molecules in the target material and in certain embodiments be transferred to other nuclear spins in the target material.

In some embodiments, an additional compound can be added to the solution. The additional compound can undergo a chemical reaction with the polarized compound. The additional compound can be selected such that a product of the chemical reaction has polarized protons and is more soluble in the solvent. In some embodiments, the polarized protons of the product can exchange with the target material. A characteristic time for this exchange can be less than a number between 10 seconds and 100 milliseconds (e.g., the time scale can be less than 1 second).

In some embodiments, the polarized compound and solvent can be selected such that the polarized compound can have a slow rate of dissolution in the solvent (e.g., seconds to minutes, or even longer). In such embodiments, new polarized molecules are continuously added to the solution over the course of dissolution, enabling a larger window of time over which NMR spectroscopy or imaging can be performed. In this manner, a slow rate of dissolution can be beneficial.

In some embodiments, the polarized compound and solvent can be selected such that the polarized compound can have a fast rate of dissolution in the solvent (e.g., seconds to hundreds of milliseconds, or even shorter). In such embodiments, following dissolution, the temperature of the solution can be lowered to solidify the solution (e.g., into a crystalline or amorphous solid). In some embodiments, the lowered temperature can be minus 20 C or lower, more preferably at minus 80 C or lower, more preferably at liquid nitrogen temperature or lower. In some embodiments, lowering the temperature of the solution can be accomplished by placing the solution in a precooled holder (e.g., a cold finger, or the like). For example, the solution can be conveyed to the precooled holder to facilitate the rapid freezing. The resulting solid contains molecules of the compound in a polarized state, together with the target material. The composition of the solid therefore enables polarization transfer from the compound to the target molecules by spin diffusion or cross polarization protocols as detailed herein.

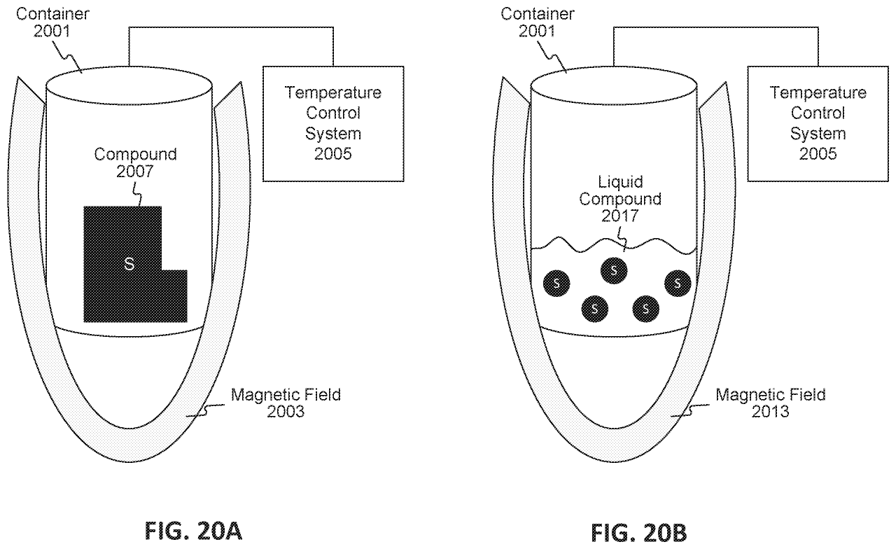

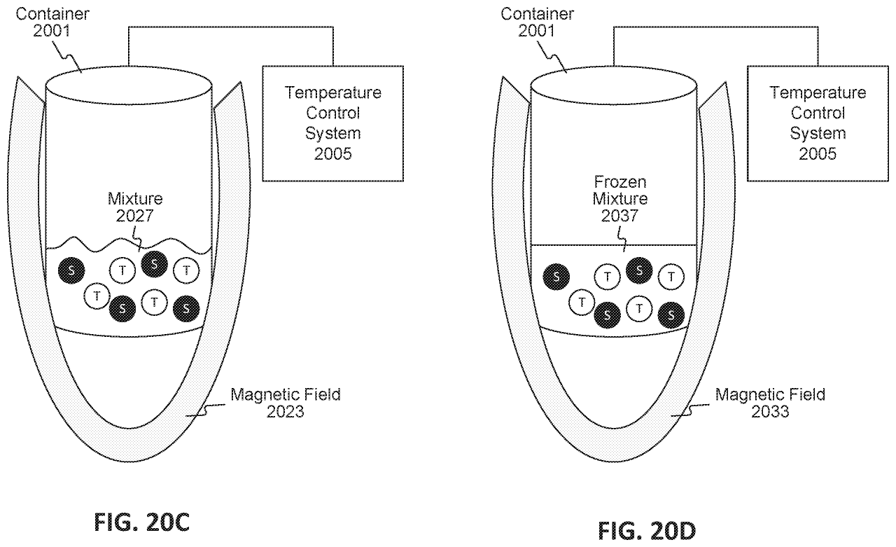

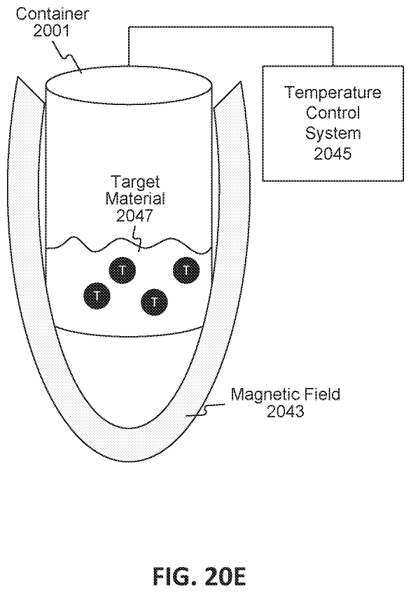

FIGS. 20A to 20E depict an exemplary process of polarization diffusion, consistent with disclosed embodiments. Each of FIGS. 20A to 20E depicts a schematic of a container in a magnetic field, consistent with disclosed embodiments. The temperature within the container is controlled by a temperature control system, consistent with disclosed embodiments. The figures depict a five-phase process from generating polarized target molecules suitable for use in an NMR or MRI investigation.

In a first phase, as shown in FIG. 20A, a polarized compound in solid form (e.g., compound 2007) can be placed in container 2001. The polarized compound can be maintained in a magnetic field (e.g., magnetic field 2003) greater than 0.1 T (as described herein, such maintenance can include exposure to low field strengths for durations on the order of seconds, depending on the intended application). The compound can be maintained at a desired temperature using temperature control system 2005. In a second phase, as shown in FIG. 20B, the polarized compound can be placed in a liquid form (e.g., liquid 2017). For example, the compound can be dissolved into a solution by a solute or melted (e.g., using temperature control system 2005). The compound can be maintained at a desired magnetic field strength 2013 during the melting or dissolving of the compound. In a third phase, as shown in FIG. 20C, a mixture (e.g., mixture 2027) of the polarized compound (indicated as filled circles) and a target material (indicated as open circles) can be formed. As described herein, the target material can be in a solid or liquid form (e.g., the target material can be dissolved into a second solution by a second solvent or melted). The mixture can be maintained at a desired magnetic field strength 2023 during mixing and at a desired temperature using temperature control system 2005. The mixture can be mixed by a processing element, as described herein. In a fourth phase, as shown in FIG. 20D, the mixture can be frozen using temperature control system 2005 (e.g., generating frozen mixture 2037). The resulting spatial, temperature, and magnetic field conditions (e.g., the short distances between molecules of the polarized compound and molecules of the target material, the low temperatures maintained by temperature control system 2005 that prolong depolarization, and magnetic field 2033) can enable diffusion of polarization between the molecules of the polarized compound and molecules of the target material. In a fifth phase, as shown in FIG. 20E, the polarized target material molecules (e.g., target material 2047—indicated as filled circles) can be separated from the molecules of the compound. For example, a second mixture can be created, the second mixture including at least some of the target molecules in the first mixture. As compared to the first mixture, the second mixture can have a reduced concentration of the molecules of the compound. For example, the concentration of molecules of the compound in the second mixture can be less than 10 mM, or less than 1 mM, or less than 0.1 mM. Suitable maximum concentration of molecules of the compound in the second mixture can be determined depending on the application (e.g., the toxicity or biocompatibility of the compound when used in vivo). In some embodiments, separation can result in the target material being in container 2001, or in another container, depending on the mechanism of separation. In some embodiments, the mixture can be maintained at a desired magnetic field strength 2043 during separation of the target material.

In some embodiments, the polarized compound can be mixed with the target material in a gas form. In such embodiments, the polarized compound in gas form can be produced by sublimation. The polarized compound can be selected to have a sublimation temperature lower than its melting point. For example, naphthalene and p-terphenyl can sublimate in lower temperatures than their melting point. Naphthalene specifically can sublimate even at very low temperatures such as below 100 C, when in contact with flowing gas. The flowing gas can be the cold gas of a cooling system used to cool the compound (e.g., nitrogen or helium gas).

In some embodiments of the invention, the polarized compound in gas form can re-solidify in a desired configuration, such as a configuration with a larger surface-to-bulk ratio. In some embodiments, the polarized compound in gas form can re-solidify in contact with the target material. For example, the target material can in a particulate form and the polarized compound can re-solidify as a coating on the particles.

Transfer Nuclear Spin Polarization to Target Material

The nuclear spin polarization of the compound can be transferred to the target material, consistent with disclosed embodiments. In some embodiments, the nuclear spin polarization can be transferred after mixing of the compound with the target material, as described herein. In some embodiments, the polarizations of nuclear spins of more than 10 picomol, preferably more than 1 nanomol, preferably more than 1 micromol, preferably more than 1 millimol, preferably more than 1 mol of nuclei of the compound are transferred to the nuclear spins of more than nanomol, preferably more than 1 micromol, preferably more than 1 millimol, preferably more than 1 mol of nuclei of the target material. Transfer can occur while the conditions for polarization transfer are met for the compound and the target material.

In some embodiments, both the compound and the target material or solvent nuclear spins have a Ti nuclear relaxation time of at least 1 second, more preferably at least 10 seconds, more preferably at least 100 seconds for 1H or other spins of interest at the temperature and magnetic field where the polarization transfer occurs. In some embodiments the magnetic field is higher than 0.05 T and the temperature is between 4K and room temperature, more preferably between 77K and 274K.

Polarization transfer to the target material can occur through multiple processes, consistent with disclosed embodiments. These processes can include zero or more intermediaries. In various embodiments, as described herein, polarization can be transferred between polarized nuclear spins in the compound and target material, using another spin species in the target material as an intermediary, or from 1H nuclear spins of the compound to 13C or 15N or other low gyromagnetic ratio spins in the target material.

Polarization transfer to the target material can include nuclear polarization transfer from the polarized nuclear spins (1H or 13C or other nuclear species and isotopes with a nuclear spin) in the compound to the nuclear spins in the target material, consistent with disclosed embodiments.

Polarization transfer to the target material can include mediation of the polarization transfer by another material, consistent with disclosed embodiments. The mediator material can include a solvent or solid matrix hosting the target material, or a mediator material (e.g., a crystalline or amorphous mediator material). Polarization transfer using mediation can include initial polarization diffusion or transfer to a solvent from the compound, followed by polarization diffusion or transfer from the solvent to the target material. For example, when a target material is deuterated to increase relaxation time and dissolved together with the polarized compound in a solidified solvent, 1H diffusion can occur from the polarized compound to 1H nuclear spins of the solidified solvent. The 1H nuclear spins of the solidified solvent can then be transferred by cross polarization to the target material molecules.

Polarization transfer to the target material can use another spin species in the target material, consistent with disclosed embodiments. In some embodiments, polarization can be transferred from the compound to the other spin species in the target material. Polarization can then be transferred from the other spin species to the target nuclear spins by cross polarization. For example, polarization can be transferred to hydrogen spins in the target material and from the hydrogen spins to 13C or 15N spins in the target material. In some embodiments, the polarization of the 1H nuclear spins in the target material and polarization transfer to other spin species can be performed repeatedly until maximal polarization in these spins is achieved.

Polarization transfer to the target material can include polarization transfer from the 1H nuclear spins of the compound to low gyromagnetic ratio spins in the target material (e.g., 13C spins, 15N spins, or the like). Such polarization transfer can be accomplished using cross polarization or low-field thermal mixing. Furthermore, polarization transfer can be performed when transferring polarization to deuterated target material or target material which contains no hydrogen molecules.

Polarization transfer to the target material can be achieved through a variety of methods, consistent with disclosed embodiments. In some embodiments, polarization transfer to the target material can be achieved using spin diffusion, in which the polarization of the nuclear spins in compound diffuses to the nuclear spins in the target material. In various embodiments, polarization transfer to the target material can be achieved using cross polarization between the nuclear spins of the compound and the nuclear spins of the target material. Such cross polarization is described in “Measuring nano-to microstructures from relayed dynamic nuclear polarization NMR,” by Pinon, Arthur C, et al. In some embodiments, polarization transfer between different nuclei spin species in a target material can be achieved using low-field thermal mixing, where the sample is quickly transported through a low magnetic field region. Such low-field thermal mixing is described in “Preparation of highly polarised nuclear spin systems using brute-force and low-field thermal mixing,” by Gadian, David G., et al.

In some embodiments, polarization transfer between different nuclei spin species in a target material can performed after transfer of polarization from the compound to a nuclei spin species in the target material and subsequent separation of the compound from the target material. For example, when the compound is a solid and the target material is a liquid target material (e.g., in a liquid phase or dissolved in a solvent), the solid compound can be filtered out prior to polarization transfer between different nuclei spin species in the target material. In some embodiments, where the target material is dissolved in a solvent, the target material can be extracted from the solvent before, during, or after polarization transfer between different nuclei spin species in a target material.

Polarization transfer parameters can be controlled to improve polarization transfer, consistent with disclosed embodiments. Such parameters can include magnetic field strength, temperature, and composition of the target material and the solvent or mediator material. In some embodiments, the polarization transfer parameters can be controlled to increase the diffusion distance from the compound to the target material/solvent. The diffusion distance from the compound can be the average distance polarization can diffuse from the compound within the relaxation time of the target material, solvent, or mediator material. The diffusion distance can be proportional to the product of the relaxation time and the square root of the diffusion coefficient of the target material, solvent, or mediator material.

In some embodiments, the diffusion distance can depend on the product of a nuclear relaxation time and spin-spin diffusion coefficient for the target material, solvent, or mediator material. The diffusion distance can be increased by increasing the product of the nuclear relaxation time and spin-spin diffusion. Techniques for increasing this product can include, as non-limiting examples, replacing fast-relaxing protons with deuterium in the target material, solvent, or mediator material; reducing the temperature; or increasing the magnetic field. As a further example, methyl group protons in amorphous solids are a prime candidate for deuteration due to their fast motion and relaxation even at liquid nitrogen temperatures.

In some embodiments, conditions (e.g., magnetic field strength; temperature; physical state, such as solid, liquid, or gas, or suspension or dissolution; addition of another compound, solvent or mediator; microwave or radiofrequency irradiation; or the like) for efficient polarization of the nuclear spins in the compound can be different from parameter values for efficient polarization transfer in the target material. Accordingly, polarization of the compound and polarization transfer to the target material can be performed under different conditions, consistent with disclosed embodiments.

As an example, many crystals have significantly longer relaxation time at higher magnetic fields and colder temperatures. Thus for the compound, if the magnetic field used during the polarization of the compound is relatively small, so that the relaxation time of the target crystal is short, transferring polarization to the target material at a higher magnetic field will allow for more time for the transfer of the polarization from the compound to the target material while limiting loss of polarization due to relaxation effect.

The change of conditions between the polarization of the compound and polarization transfer to the target material can be achieved in multiple ways. In some embodiments, the compound can be transported from an environment suitable for polarization to an environment suitable for polarization transfer. As described above, the compound can be transported in a device from a central location to another location. In some embodiments, the compound can be maintained in magnetic field greater than a minimum value between polarization and transfer of the polarization. During such maintenance, the minimum value of the magnetic field between polarization and transfer of the polarization can be greater than 10 G, more preferably 100 G, more preferably 1000 G, excluding short durations of exposure to lesser field strengths. As a non-limiting example of such a short-duration exposure, the compound can be exposed to a low magnetic field (e.g., 0.5 G or lower) for a short duration (e.g., less than 10 seconds) without depolarizing of the compound. The tolerance of short-duration exposures to lesser field strengths can be application-dependent. For example, applications requiring a high degree of polarization in the compound following transport may have a lower tolerance for such short-duration exposures than applications permitting a lower degree of polarization in the compound following transport.

In some embodiments, the compound can be mixed with the target material, as described herein, after polarization and before, during, or after transportation of the compound. In various embodiments, the compound can be mixed with the target material before polarization. In various embodiments, the compound can be maintained in place and the conditions changed from those favoring polarization to those favoring transfer of polarization (e.g., by field cycling, cooling, etc). In some embodiments, the compound can be mixed with the target material, as described herein, after polarization and before, during, or after the change in conditions to favor transfer of polarization.

Separation of Compound and Target Material

As described above with regards to FIG. 1, the target material can be separated from the compound following transfer of polarization. In some embodiments, the separation step can be performed on an original mixture of the compound and target material, resulting in a resultant mixture including target material and minimum amounts of the compound. Such separation can include removal of the compound (or of the target material from the compound) so that only trace concentrations, less than 1 mM, 1 μM, 1 nM or 1 pM, are left in the mixture or commingled with the target material. In some embodiments, such separation can include removal of at least 90% of the compound from the original mixture of compound and the target material (e.g., removal of at least 99%, 99.9%, 99.99% or more of the compound from the mixture).

In some embodiments, the compound can include non-biocompatible material, while the target material can be biocompatible. Following the polarization transfer, the polarized biocompatible target material can be separated from the non-biocompatible compound, producing a polarized biocompatible resultant mixture. The resultant mixture can be used as a magnetic resonance probe.

Separation of the compound from the target material can enable the resultant mixture to be used in applications for which the compound is unsuitable. For example, the resultant mixture could be used in magnetic resonance applications (e.g., NMR spectroscopy) where the magnetic resonance signal of the compound might otherwise mask the magnetic resonance signal of the target material, making distinguishing between the two signals difficult. As an additional example, the target material can be used to detect tissue metabolism in vitro or in vivo (e.g., hyperpolarized MM application). For such applications, toxicity, biocompatibility, or regulatory requirements may necessitate separation of the compound from the target material. Additionally, process control and result reproducibility requirements may necessitate separation of the compound from the target material.

Separation or extraction of the target molecules from the compound preferably can be performed in close proximity to the MRI or NMR device, as the relaxation time of the target material is typically short, for example on the order of a few minutes or several seconds. In some embodiments, the target material can be used (e.g., injected or probed) in the liquid state. In these cases, the mixture of compound and target material can be dissolved before the extraction of the target molecules. In some embodiments, the dissolution step can be performed by heating the mixture or by introducing an additional solvent which dissolves the target material. In various embodiments of the invention, the compound can be separated from the solution by filtering out particles of the compound (e.g., using mechanical filtration with commercial sterility filters, or the like) or by centrifuging the mixture and removing the particles of the compound.

When the compound and the target material are solutes in a solution, the compound can be removed from the solution using liquid-liquid extraction, HPLC methods (e.g. for separation of polar and non-polar molecules), introduction of an agent that undergoes a chemical reaction with the compound, or other suitable methods. For example, when the compound dissolves better in the organic phase and the target material in an aqueous phase, a liquid-liquid extraction between aqueous and organic phases can facilitate fast purification of the target material. In some embodiments, one or more quick iterations of liquid-liquid extraction can be performed, depending on the required purity of the target material. In various embodiments, liquid-liquid extraction can be performed in less than 3 minutes, more preferably in less than 1 minute, more preferably in less than 10 seconds. In some embodiments, liquid-liquid extraction can be performed as an additional purification step following other extraction and separation methods.

When compound and the target material are present in solid form, the compound can be separated out by heating the mixture to a temperature where one of the compound or target material is liquid and then separating the liquid from the solid materials. Alternatively, a solvent can be introduced that dissolves one of the compound or target material. The solvent can then be separated from the remaining solid. In some embodiments, the target material can be selected to have a long relaxation time in the solid state and can therefore retain the polarization for a long time, whether in contact with the compound or after separation.

Polarization Device

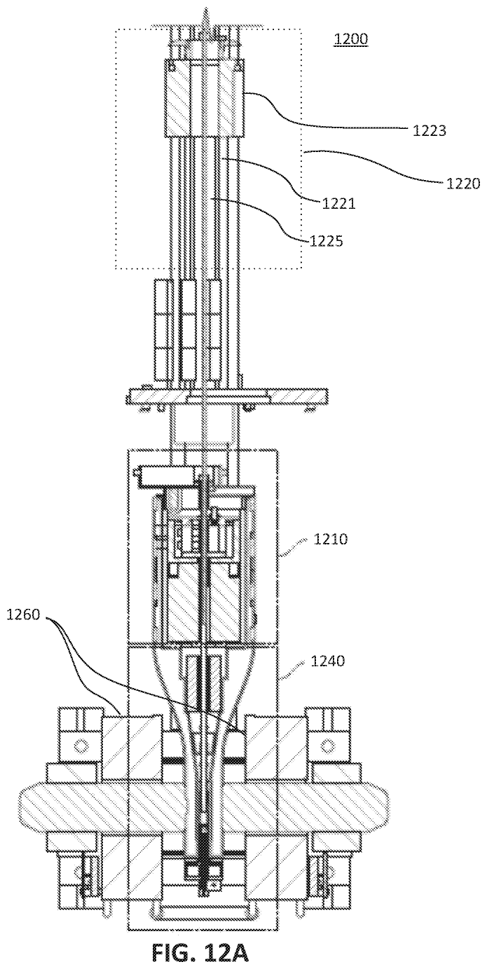

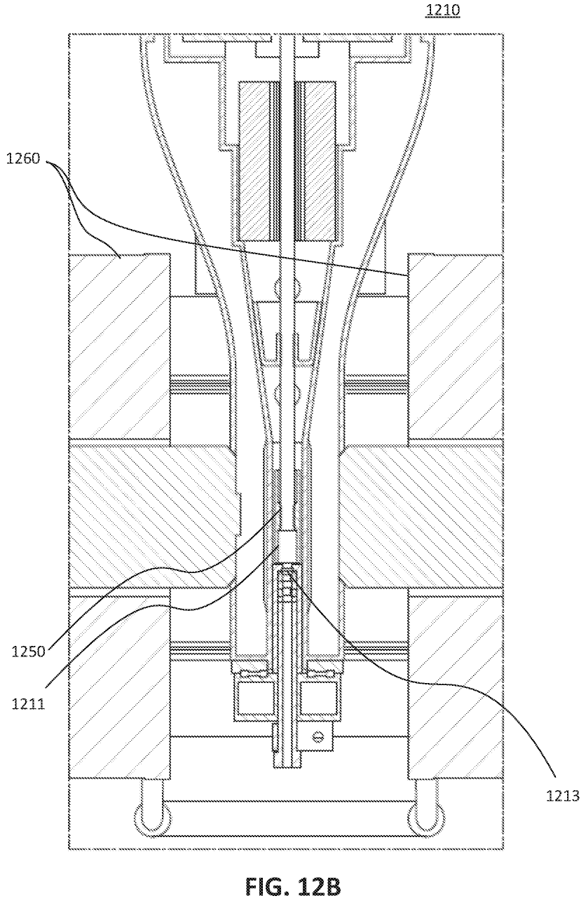

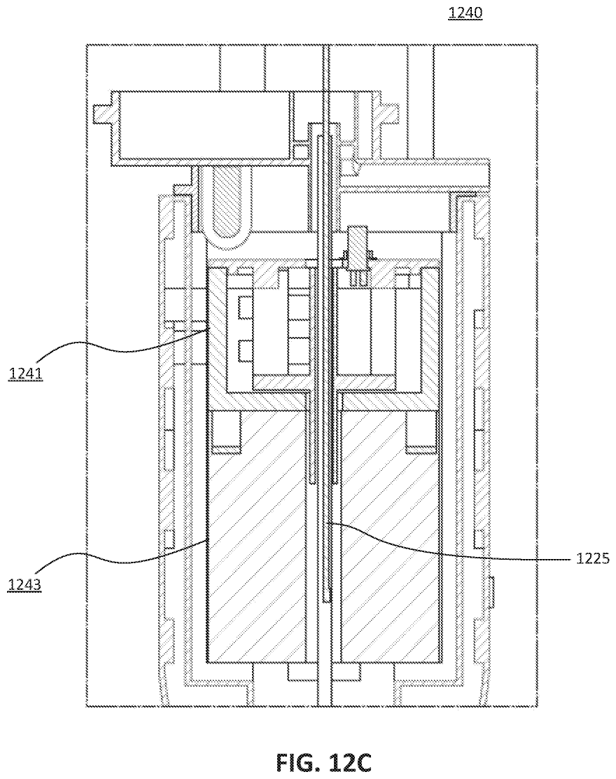

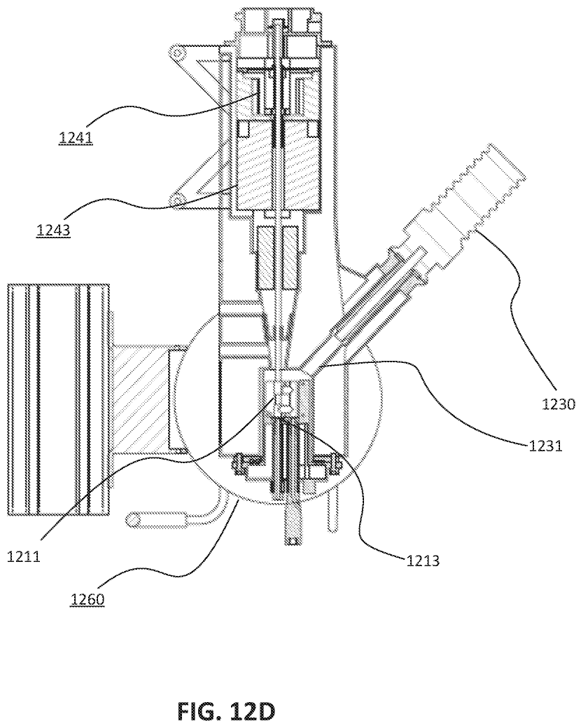

FIGS. 12A to 12D depict views of an exemplary apparatus 1200 for polarizing a compound, consistent with disclosed embodiments. In some embodiments, the compound can be or include a PETS material. Apparatus 1200 can include a polarization region 1210, an alignment and positioning system 1220, an NMR region 1240, a sample holder 1250, and a magnet 1260. Apparatus 1200 can be configured to channel light received from a light source (not shown) to a sample disposed in sample holder 1250. Apparatus 1200 can further include a cooling system configured to maintain polarization region 1210 or NMR region 1240 at a respective desired temperature.

Polarization region 1210 can be configured to enable exposure of the compound to a sequence of optical and magnetic interactions suitable for inducing polarization in the compound, consistent with disclosed embodiments. Polarization region 1210 can include a microwave cavity 1211. Microwave cavity 1211 can be one of numerous designs used to generate a homogenous microwave irradiation at a desired frequency. Microwaves can be generated by an external source (not shown). For RF frequencies typically loops or coils are used while for frequencies larger than about 2 GHz typically metallic cavities, loop-gap resonators or other variations are used. The generated microwave signal can be coupled to microwave cavity 1211 through microwave port 1213. Sample holder 1250 can be disposed within microwave cavity 1211 such that the homogenous microwave irradiation transfers polarization from electron spins to the nuclear spins during polarization of the compound, consistent with disclosed embodiments. Magnet 1260 can be disposed within polarization region 1210 around microwave cavity 1211 such that a magnetic field is produced in microwave cavity 1211. Magnet 1260 can be either a permanent magnet or electromagnet.

NMR region 1240 can be configured to enable measurement of a degree of polarization of the sample without removing the sample from apparatus 1200. NMR region 1240 can include an NMR probe 1241 and an NMR magnet 1243 tuned to measure an NMR signal from the nuclear spins of interest.

Alignment and positioning system 1220 can be configured to translate sample holder 1250 into and within apparatus 1200. In some embodiments, alignment and positioning system 1220 can enable extraction of the sample from apparatus 1200 and translation of sample holder 1250 between NMR region 1240 and polarization region 1210. In some embodiments, alignment and positioning system 1220 can include a motor 1221 configured to translate stage 1223, thereby translating sample holder 1250. Stage 1223 can be configured to enable rotation of the sample in the microwave cavity 1211. In some embodiments, the axis of rotation can be the same as the axis of translation of sample holder 1250 within apparatus 1200. In various embodiments, the axis and degree of rotation can be sufficient to form a desired angle between the molecular axis of the polarization molecules and the direction of the magnetic field established by magnet 1260. Stage 1223 can be connected to sample holder 1250 by a support member 1225. In various embodiments, translation or rotation of stage 1223 can be performed manually.

A light source (not shown) can be configured to provide optical stimulation for the compound during polarization. In some embodiments, the light source can be a source of coherent light, such as a laser. The light source can be remote from polarization region 1210. For example, light from the light source can be conveyed to the compound through an optical fiber. In some embodiments, the optical fiber can be, or be part of, support member 1225. For example, support member 1225 can be a rigid optical fiber that connects sample holder 1250 with stage 1223. Laser light generated by the light source can be applied through the optical fiber to illuminate the sample and optically polarize the compound.

The cooling system can be configured to control the temperature of the sample. In some instances, the cooling system can remove excess heat produced by optical and microwave irradiation. The cooling system can include a connection port 1230 for receiving a cooling medium, such as a gas or liquid (e.g., liquid nitrogen, cold nitrogen gas, or the like), a channel 1231 for conveying the cooling medium to microwave cavity 1211, heaters, and sensors. In some embodiments, heaters and sensors can be disposed within apparatus 1200 (e.g., in polarization region 1210 or NMR region 1240). The cooling system can include a control system configured to use heaters and sensors (and in some embodiments the cooling medium) to maintain polarization region 1210 and NMR region 1240 at desired respective temperatures or move the respective temperatures of polarization region 1210 and NMR region 1240 through desired trajectories. The cooling system also enables lowering the temperatures below room temperature to the desired temperature, including cryogenic temperatures.

Exemplary Transport Device

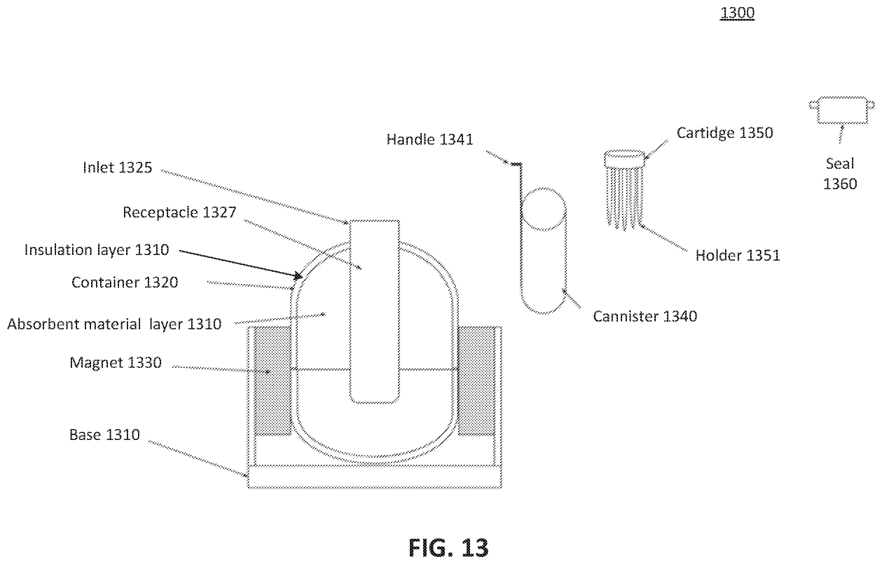

FIG. 13 describes an exemplary transport device 1300, consistent with disclosed embodiments. In some embodiments, device 1300 can include base 1310, container 1320 and magnet 1330. Device can further include canister 1340, cartridge 1350, and seal 1360.

Base 1310 can be configured to support container 1320. Magnet 1330 can be attached to base 1310 and disposed around container 1320. In some embodiments, magnet 1330 can include multiple magnets spaced around container 1320. Magnet 1330 can be configured to maintain a magnetic field with a strength between 0.1 and 4 Tesla within container 1320 (or within receptacle 1327 in container 1320) when container 1320 is placed within magnet 1330 on base 1310.

Container 1320 can include insulation layer 1321, absorbent material layer 1323, inlet 1325, and receptacle 1327. Insulation layer 1321 can be configured to insulate the inside of container 1320 from the outside environment. Insulation layer 1321 can be any suitable insulation material. Absorbent material layer 1323 can be configured to absorb a liquid coolant. For example, absorbent material layer 1323 can be suitable for absorbing liquid nitrogen or a similar cryogenic liquid. Receptacle 1327 can be a void formed in absorbent material layer 1323 below inlet 1325. In some embodiments, the void can be cylindrical. Inlet 1325 can permit access through insulation layer 1321 to the inside of container 1320.

Container 1320 can be configured to permit a liquid coolant (e.g., liquid nitrogen), to be being poured into the receptacle 1327 and absorbed into the absorbent material layer 1323. So long as sufficient liquid coolant remains, the temperature within the receptacle 1327 will approximate the temperature of the liquid coolant. In some embodiments, container 1320 can be a cryogenic dry shipper container, such as a cryostat (e.g., a Dewar, vacuum flask, or the like).

Cannister 1340 can be configured to support cartridge 1350 within receptacle 1327. In some embodiments, cannister 1340 can include a handle 1341 enabling cannister 1340 to be placed within and removed from receptacle 1327 through inlet 1325. Cartridge 1350 can be configured to hold one or more holders 1351. Cartridge 1350 can be configured and arranged such that each holder 1351 can be separately removable from cartridge 1350. Each holder 1351 can be configured to hold a sample of a polarized compound. Cartridge 1350 can be configured to within the canister and lowered into container 1320. Seal 1360 can be configured to seal the container 1320 and prevent evaporation of the coolant.

In some embodiments, device 1300 may not include base 1310. In such embodiments, magnet 1330 may be disposed within container 1320. Magnet 1330 may be disposed around receptacle 1327. In some embodiments, absorbent material layer 1323 may be disposed between magnet 1330 and receptacle 1327. In various embodiments, absorbent material layer 1323 may be disposed between magnet 1330 and an inner surface of insulation layer 1321. In such embodiments, for example, receptacle 1327 may be a void defined at least in part by the inner surface of magnet 1330.

Exemplary Polarization Transfer Systems

As disclosed herein, polarization from a polarized compound can be transferred to a target material. Included in or associated with the transfer can be processes of increasing the surface area of one or more of the compound and the target material, mixing the target material with the compound, and separating the compound and target material following transfer of polarization. In some embodiments, one or more systems can perform these processes.

Polarization Transfer Devices

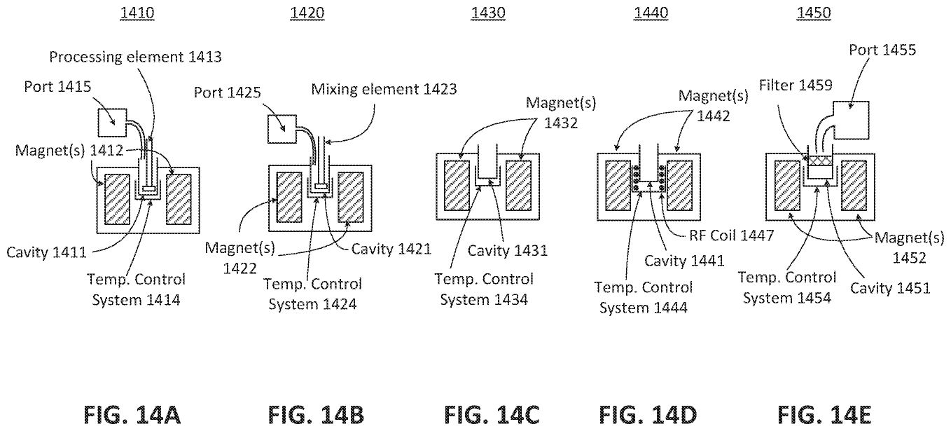

FIGS. 14A to 14E depict exemplary components collectively capable of transferring polarization from a polarized compound to a target material and separating the compound and target material. The components can be realized in one or more devices. The components include processing component 1410, mixing component 1420, diffusion component 1430, cross-polarization component 1440, and separation component 1450. In some embodiments, each of these components can be in a separate device. In various embodiments, two or more of these components can be combined into a single device.

FIG. 14A depicts an exemplary processing component 1410 configured to increase the surface area of a compound, consistent with disclosed embodiments. In some embodiments, processing component 1410 can be configured to increase the surface area of the compound by pulverizing the compound. Processing component 1410 can be configured to reduce polarization and material loss during processing of the compound. Processing component 1410 can include cavity 1411, which can be configured to hold the compound (or a holder containing the compound such as an NMR tube, holder 1351, or the like), and processing element 1413, which can be configured to increase the surface area of the compound. Processing component 1410 can further include magnet 1412 and temperature control system 1414.

Processing element 1413 can be a motorized rotating head, a mortar and pestle, a mill (e.g. a ball mill, planetary mills, or the like). Processing element 1413 can be configured to process the compound inside the magnetic field. Accordingly, in some embodiments, processing element 1413 may not include magnetic components. Alternatively, processing element 1413 can be configured to use magnetic compounds that interact with an applied magnetic field to facilitate processing. For example, processing element 1413 can be configured to interact with an applied AC magnetic field to pulverize the compound (e.g., processing element 1413 can be a magnetic cryogrinder). Processing element 1413 can be configured to process the compound into particles having a median size smaller than 1 mm\hat{ }3, more preferably smaller than 100000 um\hat{ }3, more preferably smaller than 1000 um\hat{ }3, more preferably smaller than 1 um\hat{ }3. Processing element 1413 can include or receive instructions from a control system to ensure the repeatable operation of the instrument and precise timing and control of the pulverizing head.

Magnet 1412 can be configured to generate a magnetic field in cavity 1411 during processing of the compound. Magnet 1412 can be a permanent magnet or electromagnet. Magnet 1412 can be disposed around cavity 1411. Magnet 1412 can include multiple magnets. Magnet 1412 can be configured to generate a magnetic field in cavity 1411 of at least 10 G, more preferably at least 100 G, more preferably at least 1000 G, more preferably at least 10000 G. The magnetic field strength can be selected to preserve the polarization of the compound during processing of the compound. In some embodiments, a minimal electric field strength may be preserved during processing. In some embodiments, this minimal electric field strength can be maintained using a rotating magnetic field. In such embodiments, magnet 1412 can be an electromagnetic configured to provide the rotating magnetic field.

Temperature control system 1414 can be configured to maintain the temperature of cavity 1411 during pulverization. In some embodiments, temperature control system 1414 can include a cryostat (e.g., using liquid nitrogen, a cold gas flow system, or heating or refrigeration components). The cryostat can include a temperature sensor and controller configured to maintain the cavity at a desired temperature or move the temperature of the cavity along a desired trajectory. In addition, temperature control system 1414 can be configurable to heat the compound. For example, temperature control system 1414 can include a heater for heating cavity 1411. In some embodiments, temperature control system 1414 can maintain the temperature in cavity 1411 below −20° C., more preferably below −100° C., more preferably below −150° C. during the pulverization. Such low temperatures can prolong the relaxation time of the compound nuclear spins and make the compound more brittle and thus easier to grind into fine particles.

In some embodiments, processing component 1410 can include port 1415 to introduce the target material to the compound before or during the pulverization to facilitate mixing and/or pulverization of the target material. In some embodiments, processing component 1410 can be configured to include a pump (not shown) for introducing the target material to the cavity. In various embodiments, the target material can be introduced manually.

In some embodiments, processing component 1410 can be configured to flush cavity 1411 with an inert gas after loading of the compound or target material in the cavity. Processing component 1410 can be configured to maintain an inert atmosphere in cavity 1411 throughout the mixing process.

FIG. 14B depicts an exemplary mixing component 1420 configured to mix a compound and a target material, consistent with disclosed embodiments. In some embodiments, a compound can be introduced to mixing component 1420 after pulverization (e.g., using processing component 1410). As described above, the target material can be a liquid target material or a solid target material. In some embodiments, a solid target material can be suspended in a mediator solvent. Mixing component 1420 can include a cavity 1421 configured to hold the pulverized compound and at least one magnet (e.g. magnet(s) 1422). In some embodiments, cavity 1421 can be configured to hold a tube (e.g., an NMR tube or the like) such as holder 1351. The at least one magnet can be a permanent magnet or electromagnet. The at least one magnet can be disposed around cavity 1421. The at least one magnet can be configured to generate, within the cavity, a magnetic field of at least 10 G, more preferably at least 100 G, more preferably at least 1000 G, more preferably at least 10000 G. Mixing component 1420 can further include mechanical mixing apparatus 1423. Mechanical mixing apparatus 1423 can be configured to improve the homogeneity of mixture of the compound and target material.

Similar to processing component 1410, in some embodiments mixing component 1420 can be configured to maintain an inert atmosphere in cavity 1421 during processing. In some embodiments, mixing component 1420 can be configured to include a pump (not shown) for introducing the target material to the cavity. In various embodiments, the target material can be introduced manually. In some embodiments, mixing component 1420 can contain an instrument for applying pressure on the compound before or after the introduction of the target material (e.g., a mechanical press, plunger, piston, syringe pump or the like). As described herein, such pressure can reduce the void sizes between particles in the compound and improve the contact with the target material. In some embodiments, mechanical mixing apparatus 1423 can provide this functionality. In some embodiments, a separate element can provide this functionality.

In some embodiments, mixing component 1420 can be configured to include a temperature control system. When using a liquid target material (e.g., a liquid phase target material or a target material dissolved in a solvent), the liquid target material may be introduced at a temperature above the freezing point of the liquid target material. In some embodiments, the temperature of the compound prior to introduction of the liquid target material may be below the freezing point of the liquid target material. According, temperature control system 1424 can be configurable to heat the compound. For example, temperature control system 1424 can include a heater (not shown) for heating cavity 1421. Temperature control system 1424 can also be configurable to maintain cryogenic temperature of the compound. In some embodiments, temperature control system 1424 can include a cryostat (e.g., using liquid nitrogen, a cold gas flow system, or heating or refrigeration components). The cryostat can include a temperature sensor and controller configured to maintain the cavity at a desired temperature or move the temperature of the cavity along a desired trajectory. In some embodiments, the cryostat can be a Dewar.

FIG. 14C depicts an exemplary diffusion component 1430 configured to enable transfer of polarization to the target material by spin diffusion, as described herein. Diffusion component 1430 can include cavity 1431, at least one magnet (e.g., magnet 1432), temperature control system 1434, and a monitoring system (not shown). Cavity 1431 can be configured to hold a mixture of the compound and the target material, consistent with disclosed embodiments. In some embodiments, cavity 1431 can be configured to hold a tube (e.g., an NMR tube or the like) such as holder 1351. The tube can be configured to hold the mixture of the compound and the target material. The at least one magnet can be disposed around cavity 1421. The at least one magnet can be configured to maintain a magnetic field in cavity 1431. The at least one magnet can be a permanent magnet or electromagnet. The at least one magnet can be configured to maintain a magnetic field in cavity 1431 of at least 100 G, more preferably at least 1000 G, more preferably at least 10000 G. The magnetic field strength can be selected to preserve the polarization of the compound or target material during transfer of polarization.

Temperature control system 1434 can be configured to maintain the temperature of cavity 1431 during polarization transfer. The temperature can be maintained at a setpoint or may follow a trajectory. The temperature can be below −20° C., more preferably below −100° C., more preferably below −150° C. In some embodiments, temperature control system 1434 can be configured and arranged similar to temperature control system 1414.

The monitoring system can be configured to enable detection and monitoring of polarization signals (e.g., from the compound or target material) during or after polarization transfer. In some embodiments, the monitoring system can include an NMR probe and spectrometer. In some embodiments, diffusion component 1430 can be implemented using an NMR spectrometer or MRI scanner. Similar to processing component 1410, in some embodiments diffusion component 1430 can be configured to maintain an inert atmosphere in cavity 1431 during processing.

FIG. 14D depicts an exemplary cross-polarization component 1440 configured to enable transfer of polarization to the target material by cross-polarization, as described herein. Through cross-polarization, polarization can be transferred to desired spin species in the target material. In some embodiments, the polarization can be transferred from nuclear spin species in the target material or solvent. Cross-polarization component 1440 can include cavity 1441 configured to hold a mixture of the compound and a target material. In some embodiments, cavity 1441 can be configured to hold a tube (e.g., an NMR tube or the like) such as holder 1351. The tube can be configured to hold the mixture of the compound and the target material. Cross-polarization component 1440 can further include at least one magnet (e.g., magnet 1442), a radiofrequency wave generator, a monitoring system, and temperature control system 1444.

The at least one magnet can be configured to generate a magnetic field in cavity 1441. Magnet 1442 can be a permanent magnet or electromagnet. Magnet 1442 can generate a magnetic field in cavity 1441 of at least 10 G, more preferably at least 100 G, more preferably at least 1000 G, more preferably at least 10000 G, to preserve polarization of the compound or target material during transfer of polarization. In some embodiments, magnet 1442 can generate a magnetic field in cavity 1441 that is sufficiently homogeneous to enable magnet 1442 to achieve an NMR spectrum linewidth of the 1H nuclear spins, as defined by the Fourier transform of the free induction decay, no larger than 5 MHz, more preferably no larger than 500 kHz, more preferably no larger than 100 KHz.

The radiofrequency wave generator can be configured to perform polarization transfer between nuclear species disposed in cavity 1441. In some embodiments, the radiofrequency wave generator can include one or more radiofrequency coils 1447. Radiofrequency coils 1447 can be configured and arranged to have at least two resonance frequencies. One of the resonance frequencies can be matched to a frequency of the currently polarized nuclear spin species. Another of the resonance frequencies can be matched to a frequency of the nuclear spin species to which the polarization is to be transferred. The radiofrequency wave generator can also include an RF signal generator and amplifier for generating a sequence of radiofrequency emissions. In some embodiments, the RF generator and amplifier can able generation of at least one of RF pulses, RF amplitude sweeps or modulations, or RF frequency sweeps or modulations. The generated sequence of radiofrequency emissions can depend on the chosen polarization transfer sequence and implementation. For example, the radiofrequency wave generator can be configured to generate a cross polarization sequence, an INEPT sequence (or modification thereof), a pulsed Hartmann-Hahn-type sequence, or another suitable rf-based sequences for transferring polarization between nuclear spins species in solids or liquids. In preferred embodiments, several cross-polarization steps are used with time intervals between 0.1-100 seconds to improve the polarization transfer efficiency.

Temperature control system 1444 can be configured to maintain the temperature of cavity 1441 during polarization transfer. The temperature can be maintained at a setpoint or may follow a trajectory. The temperature can be below minus 20° C., more preferably below minus 100° C., more preferably below minus 150° C. In some embodiments, temperature control system 1444 can be configured and arranged similar to temperature control system 1414.

The monitoring system can be configured to enable detection and monitoring of polarization signals (e.g., from the compound or target material) during or after polarization transfer. In some embodiments, the monitoring system can be configured with NMR detection capabilities on at least one of a frequency of the currently polarized nuclear spin species or a frequency of the nuclear spin species to which the polarization is to be transferred. The monitoring system can include an NMR probe and spectrometer. In some embodiments, cross-polarization component 1440 can be implemented using an NMR spectrometer or MM scanner. Similar to processing component 1410, in some embodiments cross-polarization component 1440 can be configured to maintain an inert atmosphere in cavity 1441 during processing.

In some embodiments, cross-polarization component 1440 can include a conveyor system (not shown) configured to produce polarization transfer using the low-field thermal mixing effect. In some embodiments, the conveyor system can be configured to transport a frozen mixture of the compound and the target material through a low-magnetic-field region (e.g., a region with a magnetic field between 0.5 G and 400 G). Transporting the frozen mixture can include a controlled translation or uncontrolled translation (e.g., dropping) of the frozen mixture through the low-magnetic-field region.

FIG. 14E depicts an exemplary separation component 1450 configured to separate the target material from the compound, as described herein. In some embodiments, separation component can perform at least one of dissolution, extraction or separation of the compound or target material. Separation component 1450 can include cavity 1451, a pump (not shown), at least one magnet (e.g., magnet 1452), and temperature control system 1454. Cavity 1451 can be configured to hold a mixture of the compound and target material. In some embodiments, cavity 1451 can be configured to hold a tube (e.g., an NMR tube or the like) such as holder 1351. The tube can be configured to hold the mixture of the compound and the target material. In some embodiments, cavity 1451 can be sized to accommodate the mixture and a solvent for dissolving the target material (or to accommodate a tube sized in such a manner). A volume of the solvent can be greater than the volume of the mixture (e.g., one to ten times more than the volume or the mixture, or greater). The pump can be configured to introduce a dissolution fluid (e.g., a solvent) to cavity 1451 (e.g., through port 1455) and extract the resulting solution.

The at least one magnet can be configured to generate a magnetic field in cavity 1451. Magnet 1452 can be a permanent magnet or electromagnet. Magnet 1452 can generate a magnetic field in cavity 1451 of at least 10 G, more preferably at least 100 G, more preferably at least 1000 G, more preferably at least 10000 G, to preserve polarization of the target material during separation.

Temperature control system 1454 can be configured to maintain the temperature of cavity 1451 during separation of the target material. The temperature can be maintained at a setpoint or may follow a trajectory. The temperature can be below minus 20° C., more preferably below minus 100° C., more preferably below minus 150° C. In some embodiments, the temperature can be selected to enable a solvent to dissolve the target material without freezing. As the mixture might be at a colder temperature following the polarization transfer or cross polarization, temperature control system 1454 may be configured to heat cavity 1451 before the introduction of the solvent. In some embodiments, temperature control system 1454 can be configured to pre-heat the solvent to a temperature greater than 20 centigrade prior to introduction of the solvent to the mixture. In some embodiments, temperature control system 1454 can be configured and arranged similar to temperature control system 1414.

In some embodiments, separation component 1450 can include filter 1459 for separating the compound from a solution containing the target material. In some embodiments, filter 1459 can be configured to filter particles of the compound (or the target material) from a solution of the target material (or the compound) dissolved in a solvent. In various embodiments, filter 1459 can be a sterile filtration membrane. Filter 1459 can be configured to remove particles above 1 um diameter, more preferably above 200 nm diameter, more preferably above 100 nm diameter, more preferably above 50 nm diameter or even smaller sizes. Separation component 1450 can be configured to pass a solution containing the target material (or compound) and suspended particles of the compound (or target material) through filter 1459. In this manner, in some embodiments, separation component 1450 can be configured to separate the compound from the target material.

Exemplary Polarization Transfer System

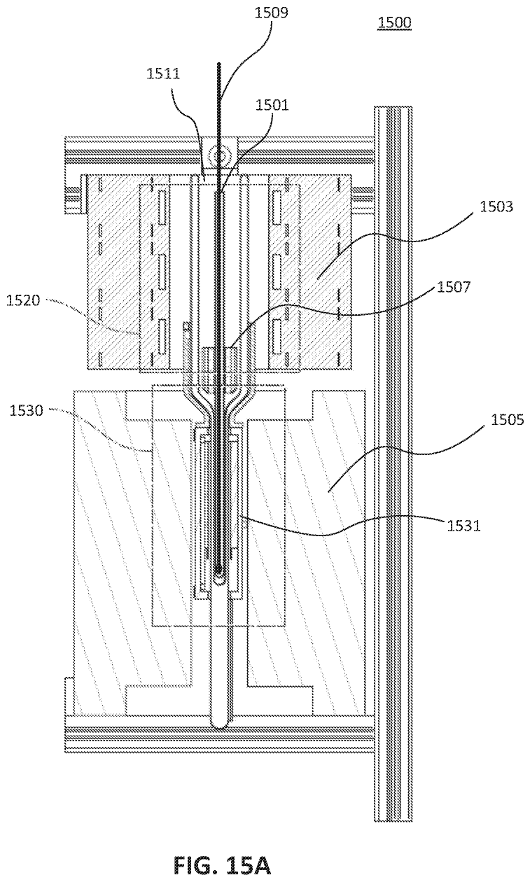

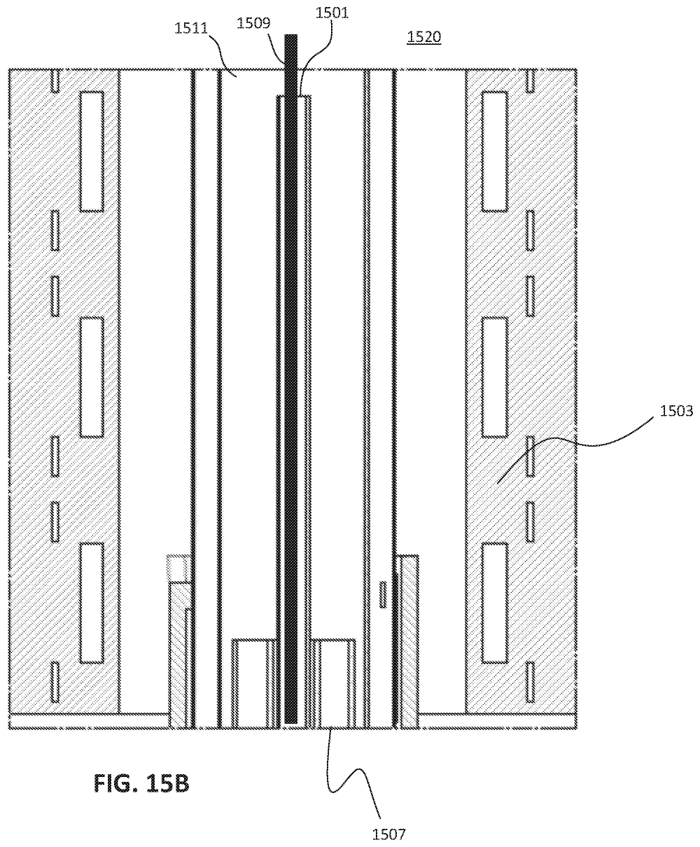

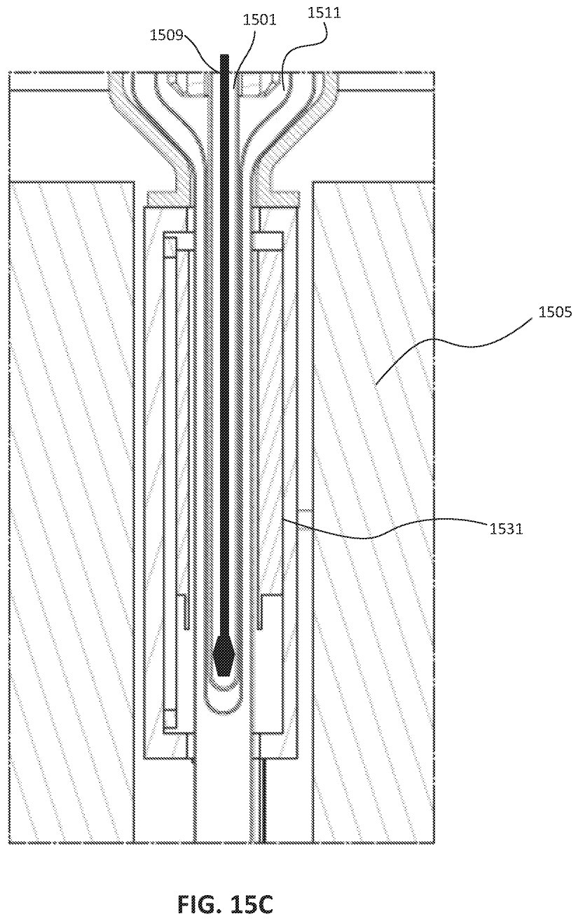

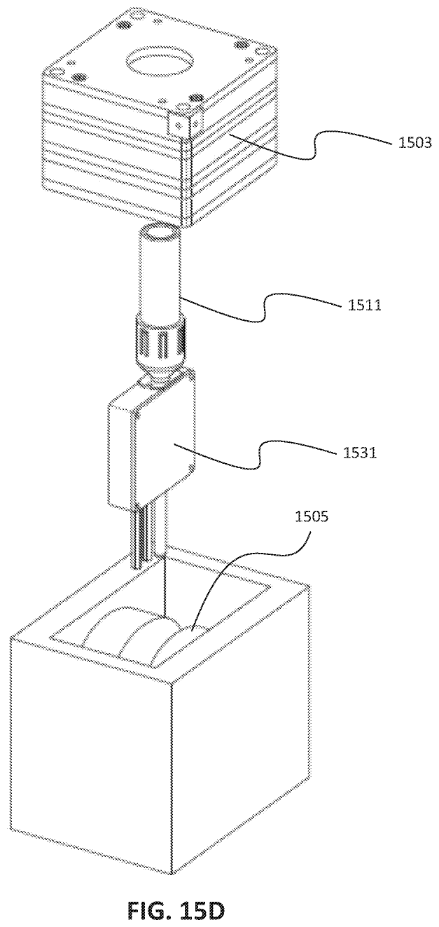

FIGS. 15A to 15C depict views of an exemplary polarization transfer system, consistent with disclosed embodiments. In some embodiments, exemplary polarization transfer system 1500 can incorporate the functionality of at least processing component 1410, mixing component 1420, diffusion component 1430, cross-polarization component 1440, and separation component 1450, as described above with regards to FIGS. 14A to 14E. Polarization transfer system 1500 can include container 1501, magnet array 1503, magnet 1505, stages 1507, and processing element 1509. Polarization transfer system 1500 can also include a temperature control system.

The temperature control system can include a cryostat 1511. In some embodiments, cryostat 1511 can be connected to at least one port for introducing a coolant medium (e.g., a cryogenic liquid, cold gas, or the like). In various embodiments, cryostat 1511 can be open at the top so that a coolant can be introduced into cryostat 1511. Cryostat 1511 can be configured to receive container 1501 and can be disposed within magnet array 1503 and magnet 1505. Container 1501 can be configured to hold the compound or a mixture of the compound and the target material. For example, container 1501 can be an NMR tube. Polarization transfer system 1500 can be configured to allow container 1501 to be translated vertically in cryostat 1511. In some embodiments, stages 1507 can be configurable to maintain the container 1501 at one of two or more positions in cryostat 1511 (e.g., by adjusting, clamping, or releasing stages 1507) or to enable extraction of container 1501 from polarization transfer system 1500. The two or more positions can include a lower position and an upper position. Cryostat 1511 can be filled with a liquid coolant (e.g., liquid nitrogen or the like) to maintain a selected temperature during the processing of the compound. Cryostat 1511 can be filled with a small amount of coolant, such that the coolant rises to the level of the bottom position. Cryostat 1511 can be filled with a larger amount of coolant, such that the coolant rises to the level of the top position. In this manner, the coolant can directly cool the bottom position, or can directly cool the bottom position and the top position. In some embodiments, a heater can be positioned around container 1501 between the top position and the bottom position. In this manner, a temperature of the cryostat 1511 in the region of the top position can be maintained separate from a temperature of the cryostat 1511 in the region of the bottom position.

A first one of the two or more positions in cryostat 1511 can correspond to preparation region 1520. Mixing, processing, and separation can be performed in preparation region 1520. Magnet array 1503 can be disposed around preparation region 1520. Magnet array 1503 can be a Halbach magnet array. In some embodiments, processing element 1509 can be disposed inside container 1501 while container 1501 is positioned in preparation region 1520. Processing element 1509 can be pressed into the compound to pulverize the compound. In some embodiments, processing element 1509 can be a non-magnetic rod (e.g., a glass rod). Polarization transfer system 1500 can be configured with a motor for pulverizing the compound using rod. Polarization transfer system 1500 can be configured to perform the pulverization for a duration between 10-100 seconds.

Polarization transfer system 1500 can be configured for introducing liquids into container 1501. For example, polarization transfer system 1500 can include a port (not shown) for introducing liquids into container 1501. As an additional example, container 1501 can be open at the top for introduction of the liquid material into container 1501. For example, a liquid can be introduced into container 1501 through an open top of container 1501 using a suitably shaped syringe.

Processing element 1509 can be configured to mix the pulverized compound with a liquid target material, consistent with disclosed embodiments. Polarization transfer system 1500 can be configured to perform the mixing for a duration between 10-100 seconds. In some embodiments, processing element 1509 can be used to compress the mixture of the polarized pulverized compound and the liquid target material.

Processing element 1509 can be configured to mix the pulverized compound with a solvent for extracting the target material, consistent with disclosed embodiments. As a nonlimiting example, following spin diffusion or cross polarization, container 1501 can be returned from polarization transfer region 1530 to preparation region 1520. In this example, a solvent can be introduced to container 1501 through a port or through an open top of container 1501. In some embodiments, polarization transfer system 1500 can be configured to mix the solvent and the mixture using processing element 1509 for a predetermined duration (e.g., 1-10 seconds). The stages 1507 can then be adjusted or released such that container 1501 can be brought out of the first cavity, to permit the mixture to be measured in an external NMR or MRI spectrometer.

A second one of the two or more positions in cryostat 1511 can correspond to polarization transfer region 1530. Spin diffusion and cross-polarization can be performed in polarization transfer region 1530. Magnet 1505 (e.g., a spin magnet) can be disposed around polarization transfer region 1530. In some embodiments, container 1501 can be positioned in polarization transfer region 1530 following performance of pulverization and mixing in preparation region 1520. For example, when container 1501 is an NMR tube, the NMR tube can be lowered further into cryostat 1511. In some embodiments, container 1501 can be positioned in polarization transfer region 1530 by releasing stages 1507 and clamping container 1501 in place once the mixture is properly disposed within polarization transfer region 1530 (e.g., when the mixture is disposed in NMR probe 1531).

As depicted in FIGS. 15A to 15D, polarization transfer region 1530 can differ in shape from preparation region 1520. For example, polarization transfer region 1530 can be narrower than preparation region 1520. In some embodiments, a clearance between an inner wall of cryostat 1511 can be less inside polarization transfer region 1530 than preparation region 1520. In some embodiments, the greater clearance in preparation region 1520 can accommodate vibrations or other disturbances arising from the action of processing element 1509.

In some embodiments, polarization transfer region 1530 can include NMR probe 1531. NMR probe 1531 can be configured to detect or monitor a hyperpolarization signal of the mixture during or after the polarization transfer, or during or after the cross polarization, using small flip angles (e.g. 1-3 degree flip angles). Such detection and monitoring can be accomplished without a large detrimental effect on the polarization, consistent with disclosed embodiments. The sample in container 1501 may be disposed within NMR probe 1531 when container 1501 is in the second position. In some embodiments, magnet 1505 can be arranged around NMR probe 1531. Magnet 1505 can be configured to supply a 0.5 T magnetic field with 100 ppm homogeneity throughout the sample. In some embodiments, polarization transfer region 1530 can be configured with a temperature and magnetic field selected to permit polarization to diffuse from the compound to the target material. As a non-limiting example, such polarization diffusion can be performed for a predetermined duration (e.g., for a duration ranging from 10 to 400 seconds).

NMR probe 1531 can include dual frequency RF coils, consistent with disclosed embodiments. The dual-frequency RF coils can be tuned to differing resonance frequencies associated with different spin species. As a non-limiting example, the dual frequency RF coils in the NMR probe can be configured to perform a double spin-locking cross polarization sequence from one nuclear spin species in the target material or solvent to the desired spin species in the target material. In some embodiments, the dual-frequency RF coils can be tuned to the resonance of 1H and 13C nuclear spins.

Additional Exemplary Polarization Transfer System

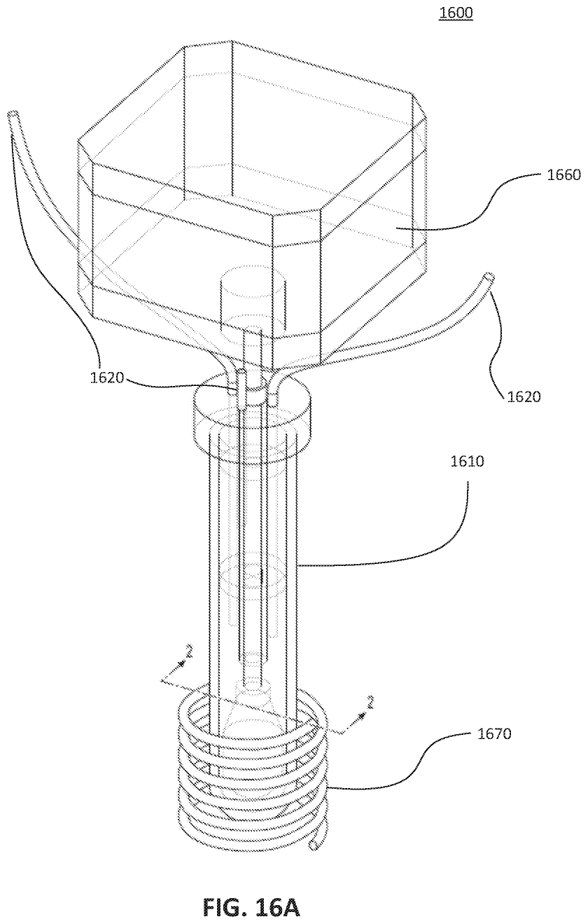

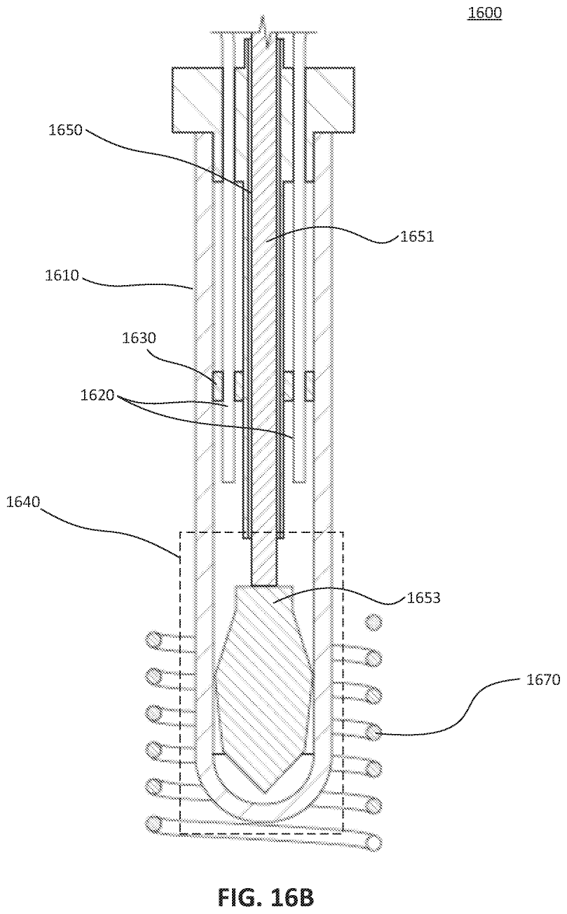

FIGS. 16A and 16B depict views of an alternative exemplary polarization transfer system 1600, consistent with disclosed embodiments. In some embodiments, exemplary polarization transfer system 1600 can incorporate the functionality of at least processing component 1410, mixing component 1420, diffusion component 1430, cross-polarization component 1440, and separation component 1450 described above with regards to FIGS. 14A to 14E. Polarization transfer system 1600 can include a container 1610. Container 1610 can be a non-magnetic container (e.g., a glass container). Container 1610 can include one or more lines 1620 for introducing and removing the target material, a solution containing the target material, or a solvent for separating the target material from a mixture of the target material and the compound. In some embodiments, a position within container 1610 of the distal portion of each of lines 1620 can be secured. For example, as shown in FIG. 16, the distal portions of lines 1620 can pass through support 1630. Support 1630 can be configured to maintain the position and location of the distal ends of each of lines 1620 in container 1610. Container 1610 can be configured with processing region 1640. Processing region 1640 can be surrounded by a magnet (not shown). The magnet can be a permanent or electromagnet. The magnet can be configured to generate a magnetic field in processing region 1640. In some embodiments, the magnetic field can have a strength of between 0.05 T and 3 T.

Polarization transfer system 1600 can include a temperature control system. In some embodiments, the temperature control system can use a gas flow into container 1610 to control the temperature in processing region 1640. In some embodiments, polarization transfer system 1600 can include a gas inlet port and a gas outlet port or a vent. The gas inlet port and the gas outlet port or vent can be arranged at the top of polarization transfer system 1600. Gas can be flowed into container 1610 through the gas inlet port and exhausted through the outlet port or vent. The gas can be inert gas and can be provided to displace undesired species (e.g., oxygen or the like) in container 1610. The gas can be colder than the inner container to cool the inner container or hotter than the inner container to heat the inner container. In this manner, flow of different gases at varying temperatures into container 1610 can enable temperature control in the canister. The temperature, heat capacity, and flow rate of the gases can be selected to achieve the desired degree and rate of heating or cooling. In some embodiments, the gas can be flowed through container 1610 using lines 1620.

Polarization transfer system 1600 can include processing element 1650. Processing element 1650 can be or include rod 1651 with a diameter less than the inner diameter of inner container 1610. Processing rod 1651 can enter container 1610 axially through an opening at the top of container 1610. In some embodiments, processing rod 1651 and container 1610 can have a common axis. In some embodiments, processing element 1650 can include or be connected to a motor 1660. For example, motor 1660 can be connected to a proximal end of processing rod 1651. The motor can be configured to spin processing rod 1651 around its axis. In some embodiments, processing head 1653 can be attached to the distal end of processing element 1650. Processing head 1653 can be configured and shaped such that, when motor 1660 spins processing rod 1651 around its axis, processing head 1653 breaks up a compound or mixes a compound and solution disposed in processing region 1640. In various embodiments, processing rod 1651 can be moved along its axis to pulverize a compound or mix a compound and solution disposed in processing region 1640. For example, processing rod 1651 can apply pressure, compressing the mixture of pulverized compound and liquid target material following mixing.

In some embodiments, dual frequency RF coils 1670 can be disposed around processing region 1640 to enable transference of polarization to a desired spin species in the target material. The polarization can be transferred from the compound or from a solvent that was polarized by the compound.

In some embodiments, the temperature control system can be configured to maintain the temperature of container 1610 at a temperature or in a temperate range falling within minus 150° C. to minus 50° C. during pulverization (e.g., from minus 120° C. to minus 80° C.). In some embodiments, a liquid target material can be added through one or more of lines 1620 after pulverization. The temperature of container 1610 can be maintained at a temperature or in a temperate range falling within minus 200° C. to minus 100° C. during pulverization (e.g., from minus 170° C. to minus 130° C.). The mixture can solidify and allowing polarization can diffuse from the polarized compound to the target material. In some embodiments, processing element 1650 can apply pressure before or during solidification. In some embodiments, radiofrequency stimulation can be applied using dual frequency RF coils 1670 to transfer polarization to a desired spin species in the target material. In various embodiments, the temperature control system can be configured to maintain the temperature of container 1610 at a temperature or in a temperate range falling within 20° C. to 60° C. (e.g., from 30 to 50° C.) during separation of the target material from the compound. In some embodiments, a warm solvent can be injected through one or more of lines 1620, dissolving at least some of the target material, which can be carried out through the outlet for transfer to an MM or NMR spectrometer. In some embodiment, the solvent can be filtered following extraction to remove any particles of the compound, as described herein.

Pre-Polarization Mixing of Compound and Target Material

As described above with regards to FIG. 1, in some embodiments, a compound with a long relaxation time that is optimized for polarization can be polarized in a polarizer that is spatially separated from a location of use (e.g., an MRI suite). The polarized compound can then be transported proximate to the location of use and then used to polarize a target material suitable for use in the intended application. After polarization of the target material, the target material can be separated from the compound. However, the disclosed embodiments are not limited to the approach depicted in FIG. 1.

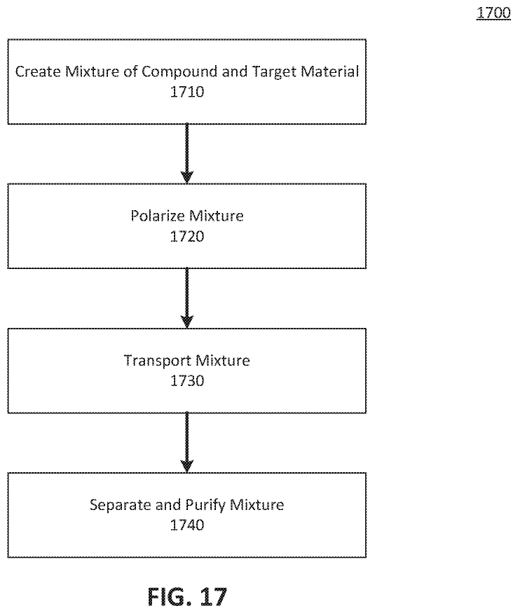

FIG. 17 depicts an exemplary process 1700 in which the compound is mixed with the target material prior to polarization. The mixture is subsequently polarized and then transported. After transportation, the target material can be separated from the compound and used. In this manner, the end-user may only be responsible for separation of the target material. Accordingly, process 1700 may enable additional centralization and resultant efficiencies in the production of polarized materials.

In step 1710 of process 1700, a mixture can be prepared. The mixture can include a compound and a target material. In some embodiments the compound can be or can include a PETS material. In various embodiments, mixing the compound and the target material can involve increasing the surface area of at least one of the compound or target material, as described with regards to FIG. 1. In some embodiments, at least one of the compound or target material can be in micro- or nanoparticle form (e.g., as a result of increasing the surface area of the compound or target material). In various embodiments, there can be contact over a large surface area between the compound and the target material.

In some embodiments, the compound and the target material can be combined to create a porous mixture. The mixture can include particles of the target material entrapped in polycrystals of the compound; particles of the target material entrapped in a single crystal or a mostly single crystal preparation of the compound; or the target material can be added to a powder of the micro- or nanoparticles of the target material.

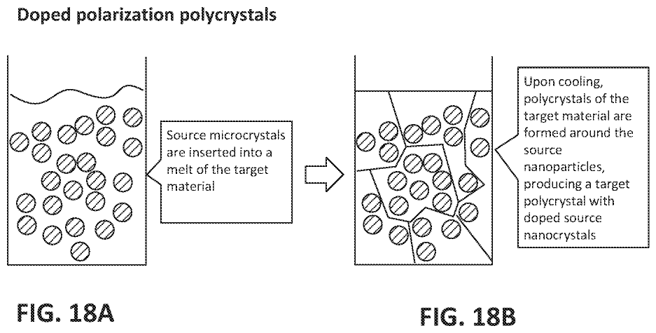

As depicted in FIGS. 18A and 18B, particles of the target material can be entrapped in polycrystals of the compound, consistent with disclosed embodiments. In some embodiments, as shown in FIG. 18A, the target material can be introduced into a melt, solution, or vapor of the compound (or can have the compound grown around the particles of the target material by another crystal growth method). As shown in FIG. 18B, particles of the target material can be overgrown by or encapsulated into the polycrystal(s). The particles of the target material can be micro- or nano-particles.

Entrapping particles of the target material in polycrystals of the compound can reduce additional preparation steps for hyperpolarizing the target material once the PETS material is hyperpolarized. For example, such entrapped can result in the desired increase in surface area, obviating the need for any pulverization of the compound or target material. Polycrystals may be grown more easily than single crystals and the volume of the compound as compared to the target material can be significantly reduced. Moreover, using polycrystals can enable production of larger mixtures, as polycrystals are more easily grown than single crystals. Entrapment can be achieved in the following non-limiting ways:

Particles of the target material can be inserted into a melt of the compound. Polycrystals of the compound can be grown from the melt. The crystals can be grown from the melt in several crystal growth methods, including rapid temperature reduction, the Bridgman growth method, Czochralski method, the cell method, or other known crystal growth methods. Advantageously, many target materials of key interest for hyperpolarized MM, such as urea, fumarate, sodium pyruvate and glucose, have a melting temperature that is higher than the melting temperature of naphthalene, and many of them are higher than the melting temperature of p-terphenyl, so that they can easily be placed into the melt in crystal form. In a certain embodiment, a plurality of structures with large surface to bulk ratio (e.g. wires, mesh, gels, thin films) coated with the target material are placed into the melt of the PETS material. These structures assist in holding the target material in place during the crystallization process of the PETS material, thereby verifying that the target material is incorporated into the PETS material and not separated during the crystallization process.

Particles of the target material can be used as seeds for growing the polycrystals from a solution. The growth parameters of the polycrystals can be controlled to improve the purity of the polycrystals and controlling the thickness and size of the polycrystals.

Particles of the target material can be used as seeds for growing the polycrystals by deposition of the compound. Crystals of high purity can be grown from vapor phase by sublimation, condensation and sputtering of the compound.

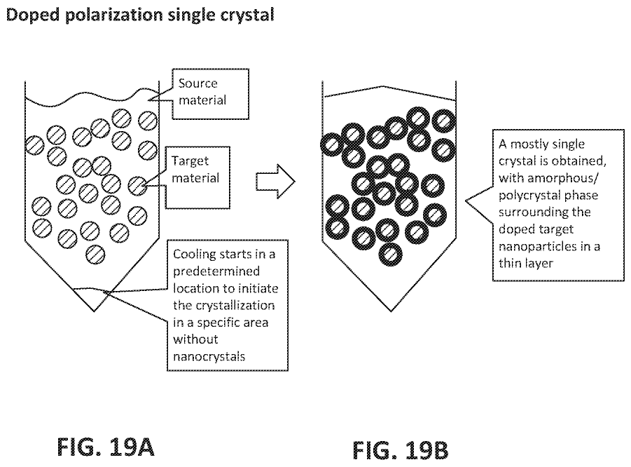

As depicted in FIG. 19A or 19B, particles of the target material can be entrapped in a single crystal or a mostly single crystal preparation of the compound, consistent with disclosed embodiments. The particles can be micro- or nanocrystals. As shown in FIG. 19A, the particles can be introduced into the crystal(s) of the compound during the growth of the compound. As shown in FIG. 19B, the crystal growth and seeding of the target material can be configured to produce one or more mostly single crystal(s) of the compound doped with the particles of the target material.

Embodiments using single crystal(s) of the compound doped with the particles of the target material can combine the efficiency of polarization of a single crystal with the large surface area of contact between the PETS material and the target material. This advantageous combination can enable achievement of a high degree of polarization in the target material (e.g., >1%, >10%, >20%). As shown in FIG. 19B, due to surface effects, the immediate vicinity of each particle may not be ordered along the single crystal structure. However, advantageously, polarization through spin diffusion between ¹H nuclear spins is relatively large due to their high gyromagnetic ratio and large density. In some embodiments, the diffusion constant can be approximately D=1000 nm²/s. Hence, on the order of 1000 seconds, the built-up polarization will have a diffusion range of around 1 um (for a single crystal of a PETS materials such as pentacene:naphthalene, the Ti time of the proton spins is significantly higher than 1000 seconds). Therefore, if a portion of a single crystal of the PETS material is within a few um of the target material, polarization of the PETS material will diffuse into the target material, thus building up the polarization.

A single crystal or mostly single crystal of the compound can be grown around a particle of the target material by means of a melt, a solution or a vapor, as described below. In some embodiments, the melt, the solution or the vapor can include polarizable molecules, such as a PETS material.

Particles of the target material can be inserted into a melt of the compound from which the single crystal(s) or the mostly single crystal(s) is/are grown. The size of the particles can be selected such that, during crystal growth, the micro- or nanoparticles will not be pushed out and can be incorporated into the crystal(s) of the compound. In a certain embodiment, a plurality of structures with large surface to bulk ratio (e.g. wires, mesh, gels, thin films) coated with the target material can be placed into the melt of the PETS material. These structures can assist in holding the target material in place during the crystallization process of the PETS material, thereby enabling verification that the target material is incorporated into the PETS material and not separated during the crystallization process.

The melt can be crystallized by several methods for producing single crystals or mostly single crystals of high purity, including the Bridgman method or the cell method. In embodiments using single crystals of pentacene:p-terphenyl, the Czochralski method or the like can be used. Advantageously, many target materials for hyperpolarized MRI (e.g., urea, fumarate, sodium pyruvate, glucose, and the like) have melting temperatures higher than the melting temperature of potential compounds (e.g., naphthalene, p-terphenyl, and the like) so that they can be inserted into the melt in crystal form.

Particles of the target material can be used as seeds for growing the single crystals from a solution. The particles can be co-doped in the solution. Embodiments combining the compound and target material in such a manner may not require a configuration change in the polarization device between the polarization of the PETS material and the polarization diffusion into the target material. Following the polarization of the target material, the compound can be separated from the target material. For example, the compound can be dissolved or sublimated by increasing the temperature of the mixture. As an additional example, the compound can be dissolved in a solution which dissolves the compound but not the target material, such as an organic solvent. Such embodiments can exploit differences in solubility between the compound and the target material (e.g., potential compounds such as naphthalene and p-terphenyl are non-polar molecules, while many potential target materials are polar molecules, resulting in substantial differences in solubility for many solvents).

In some embodiments, multiple particles of the target material can be entrapped in the same single crystal, polycrystal, or glassy solid of the compound. A number of the entrapped particles can be between 10⁵ and 10¹², or greater. In various embodiments, each particle of the target material can be individually entrapped in a single crystal, polycrystal, or glass of the compound. For example, each particle of the target material, together with the molecular single crystal, molecular polycrystal, or glassy solid of the PETS materials, in which it is entrapped, can be separate from (as opposed to formed in one piece with) other particles entrapped in other molecular single crystals, molecular polycrystals, or glassy solids of the compound.

The target material can be added to a powder of particles of the compound, consistent with disclosed embodiments. In some embodiments, the compound can be present in the form of micro- or nanoparticles of one or more porous polycrystal(s). In such embodiments, the zero-field splitting of the photo-excitable triplet states may cause an inhomogeneous broadening of the electron spin resonance due to the random orientation of the molecule with regard to an external magnetic field. Such an inhomogeneous broadening can negatively affect the polarization efficiency and/or requiring more sophisticated polarization sequences. In Takeda, Kazuyuki, K Takegoshi, and Takehiko Terao, “Dynamic nuclear polarisation by photoexcited-triplet electron spins in polycrystalline samples.” Chemical physics Letters 345. 1-2 (2001): 166-170, a method for polarizing a single polycrystalline naphthalene sample is presented, where the ISE protocol was used to sweep over the maximum of the EPR signal, thereby enabling a significant portion of the pentacene molecule alignments to be involved in the DNP process. The relevant parts of this document are incorporated into the present disclosure by reference.

In some embodiments, the compound can be present in the form of micro- or nanoparticles. Advantageously, such micro- or nanoparticles can be brought in close contact to the target material. In some embodiments, the micro- or nanoparticles can be molecular crystals. The micro- or nanoparticles can be mixed with the target material. For example, the target material can be added to a powder of the micro- or nanoparticles of the compound. In a preferred embodiment, the compound can be compressed, condensing the distances between the micro- or nanoparticles.

In some embodiments, the target material can be present in the form of one or more glassy solid(s), and the micro- or nanoparticles of the compound can be entrapped in the glassy solid(s) of the target material. In various embodiments, the target material can be present in the form of one or more single crystal(s), mostly single crystal(s), or a polycrystal(s). Preferably, the target material can be provided in the form of a solution which can be glassified by reducing the temperature, and in which the micro- or nanoparticles of the compound are suspended. For example, the micros- or nanoparticles of the compound can be suspended in a solution containing the target material. The suspension can then be frozen or glassified, as described herein. Alternatively, the micro- or nanoparticles of the compound can be packed in a dense structure, thereby producing a porous environment through which a solution of the target material can be introduced and, subsequently, frozen or glassified.

In some embodiments, each micro- or nanoparticle of the PETS material can be individually entrapped in a single crystal, polycrystal, or glass solid of the target material. Alternatively, multiple micro- or nanoparticles of the compound can be entrapped in the same single crystal, polycrystal, or glass solid of the target material.

In some embodiments, the target material can be present in the form of a solution, and the compound can be suspended in the solution of the target material. Preferably, micro- or nanoparticles of the compound can be suspended in a solution containing the target material. Alternatively, the micro- or nanoparticles of the compound can be packed in a dense structure, thereby producing a porous environment through which a solution of the target material can be introduced.

In some embodiments, the compound can be present in the form of micro- or nanoparticles and the and the target material can also be present in the form of micro- or nanoparticles. For example, at least one of the compound and the target material can be present as a powder. Preferably, the micro- or nanoparticles of the PETS material are mixed with micro- or nanoparticles of the target material. Preferably, the micro- or nanoparticles of the PETS material can be combined to form a porous polycrystalline material with the target material (for example single crystals, mostly single crystals or polycrystals of the target material) filling the void spaces of the porous PETS material. Preferably, after production of micro- or nanoparticles of the PETS material, these micro- or nanoparticles can be mixed with the target material and packed closely together. For example, such packing can yield a semi-single porous polycrystalline solid, where sufficient contact is established between the compound and target particles for the polarization to diffuse from the compound nanoparticles to the target nanoparticles. In another embodiment, an amorphous or liquid mediator is added to the nanoparticulate mixtures, filling the voids and thereby establishing contact between the nanoparticles to enable diffusion. Preferred options for the mediator are liquids which wet but do not significantly dissolve both the compound and target nanoparticles and that upon lowering the temperature freeze in a glassy state.

In step 1720 of process 1700, the mixture can be polarized. In some embodiments, the electron spins in the compound can be optically polarized and transferred to the nuclear spins in the compound. Nuclear spins in the compound can then be transferred to nuclear spins of the target material, as described with regards to FIG. 1. In various embodiments, the nuclear spins can be transferred by at least one of spin diffusion or cross polarization. In some embodiments, polarization can be transferred to the target material by at least one of cross polarization or spin diffusion while the compound is being polarized. In another embodiment, the compound can be polarized and then the polarization can be transferred to the target material by at least one of cross polarization or spin diffusion. In some embodiments, polarization of the compound and transfer of polarization to the target material can happen repeatedly. Accordingly, a very high polarization can be achieved in the target material (e.g., >1% polarization, >10% polarization).

In step 1730 of process 1700, the mixture can be transported to the destination location. Transport of the mixture can occur in a manner similar to transport of the compound, as described above with regards to FIG. 1. In certain embodiments, the mixture can be transported with the hyperpolarized nuclear spins being in the compound, with the polarization transfer performed following the transport. In other embodiments, mixture can be transported with the hyperpolarized nuclear spins being in the target material. In certain such embodiments, the separation of the target material is performed before the transport. In certain other embodiments the transport occurs before the separation of the target material.

In step 1740 of process 1700, the target material can be separated from the compound. Separation of the target material from the compound can occur as described above with regards to FIG. 1. Following the separation and purification, the target material can be used (e.g., in hyperpolarized MRI/NMR measurement).

Exemplary Applications

The disclosed embodiments can be used for applications requiring polarized nuclear spins. For example, the disclosed embodiments can be used to generate polarized target materials for use in NMR and MRI applications. In particular, the disclosed embodiments can be used in hyperpolarized magic angle spinning NMR (MAS-NMR), hyperpolarized liquid-state NMR, and hyperpolarized MM. In each application, several modifications on the system are possible, optimizing it for the application and potentially making use of the existing hardware, software and infrastructure.

Hyperpolarized Magic Angle Spinning NMR (MAS-NMR)

In MAS-NMR, the target material can be measured in the solid form at various temperatures, consistent with disclosed embodiments. In some embodiments, the target material can be assessed at temperatures lower than 20° C. Accordingly, in some embodiments, the polarization transfer system may not need to separate the target material from the compound. In various embodiments, depending on the application performed in the MAS-NMR spectrometer, the polarization transfer system may not need to perform cross-polarization step.

In various embodiments, the target material (or the mixture containing the target material) may be maintained in a magnetic field of at least 1 G between removal from the polarization transfer system and placement within the MAS-NMR spectrometer.

In some embodiments, the target material may be placed within the MAS-NMR rotor before the MAS-NMR measurements can occur. In such embodiments, the target material or mixture can be inserted into the MAS-NMR rotor before, during, or after polarization transfer. For example, the target material or mixture can be inserted into the MAS-NMR rotor before pulverization or mixing of the compound and the target material, spin diffusion or cross-polarization, or separation of the target material from the mixture (when such separation is performed). In some embodiments, at least one of spin diffusion or cross-polarization can be performed in the MAS rotor in the NMR spectrometer using the NMR magnet, probe, temperature control, rf irradiation and detection.

Hyperpolarized Liquid-State NMR

In hyperpolarized liquid-state NMR, the target material can be measured in liquid form, consistent with disclosed embodiments. In some embodiments, cross polarization may not be performed, depending on the application performed in the NMR spectrometer.

In some embodiments, the target material can be dissolved but not extracted or separated from the mixture. In some embodiments, spin diffusion, cross polarization, and separation of the target material from the source material molecules can be performed in the NMR spectrometer magnetic field. In certain embodiments, some or all of these operations can be performed using the NMR magnet, probe, temperature control, rf irradiation and detection. In some embodiments, the target material is used to amplify signal from other molecules in the NMR spectrometer, for example the injection of hyperpolarized water in deuterium oxide (D2O) to a solution containing proteins, where the exchange of protons between the hyperpolarized water and proteins enhances the NMR signal from the protein nuclear spins.

Hyperpolarized Mm

In hyperpolarized MM the target material is injected into living tissue or in vivo in a liquid form, consistent with disclosed embodiments. In some embodiments, spin diffusion, cross polarization, and separation can be performed in the MRI scanner magnetic field. In various embodiments, some or all of these steps can be performed using rf irradiation and detection functionality provided by components of the MRI scanner.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

The embodiments may further be described using the following clauses:

1. A method of forming a hyperpolarized NMR or MRI target material, the method comprising: obtaining a compound having nuclear spins, wherein the compound is selected to have, under optical radiation, electron spins exceeding 10% polarization; optically hyperpolarizing electron spins of the compound; transferring polarization from the electron spins of the compound to nuclear spins of the compound, at least in part by exposing the compound to a magnetic field; and exposing the compound to a target material before or after pulverizing the compound to increase the surface area of the compound, thereby facilitating transfer of polarization from the compound to the target material.

2. The method of clause 1, wherein the compound includes a mixture of a dopant and an additional material, and the optically hyperpolarized electron spins are intrinsic to the dopant.

3. The method of clauses 1 or 2, wherein the compound includes a doped molecular crystal.

4. The method of clause 3, wherein the molecular crystal includes at least one of naphthalene, p-terphenyl, benzoic acid, or derivatives thereof.

5. The method of clauses 3 or 4, wherein the dopant includes at least one of aromatic hydrocarbons, pentacene, tetracene, anthracene, or derivatives thereof.

6. The method of any one of clauses 1 to 5, wherein the compound is exposed to the target material before the transfer of polarization to the nuclear spins of the compound.

7. The method of any one of clauses 1 to 5, wherein the compound is exposed to the target material after the pulverization of the compound.

8. The method of any one of clauses 1 to 7, wherein: the target material comprises at least one of a liquid or a solute in a solution; exposing the compound to a target material comprises mixing the pulverized compound and the target material; and the method further comprises freezing the mixture of the pulverized compound and the target material.

9. The method of clause 1, wherein both the target material and the pulverized hyperpolarized compound are in microcrystalline form.

10. The method of clause 9, further comprising adding to the exposed compound at least one of a liquid or an amorphous material to facilitate polarization transfer between the exposed compound and the target material.

11. The method of any one of clauses 1 to 10, wherein the target material comprises at least one of urea, pyruvic acid, pyruvates, fumarate, bicarbonate, dehydroascorbate, glutamine, acetate, alpha-ketoglutarate, dihydroxyacetone, acetoacetate, lactate, glucose, ascorbic acid, zymonic acid, or derivatives thereof.

12. The method of any one of clauses 1 to 11, further comprising separating the target material from the compound and injecting the target material into biological tissue.

13. The method of any one of clauses 1 to 12, wherein the compound is in a form of molecular crystals, and wherein energizing further includes exposing the molecular crystals to microwave energy.

14. The method of any one of clauses 1 to 13, wherein the magnetic field is tuned to match a value in which an energy level of the optically hyperpolarized electron spins and the compound nuclear spins share a common resonance.

15. The method of any one of clauses 1 to 14, wherein the pulverization reduces the compound to at least one of micro particles or nano particles with a median size no larger than 0.001 mm3.

16. The method of any one of clauses 1 to 15, wherein the polarization transfer from the nuclear spins of the pulverized compound to the nuclear spins of the target material occurs via spin-diffusion by nuclei of a common species.

17. The method of clause 16, further comprising transferring polarization within the target material from protons to nuclear spins having a lower gyromagnetic ratio than a gyromagnetic ratio of the protons.

18. The method of any one of clauses 1 to 17, wherein the transferring of polarization from the electron spins of the compound to the nuclear spins of the compound occurs in a first device and the pulverization occurs in a second device, and wherein the method further comprises transferring the compound from the first device to the second device.

19. The method of any one of clauses 1 to 18, wherein at least 1 nanomole of target material is hyperpolarized.

20. The method of any one of clauses 1 to 19, wherein the pulverization increases the surface area of the pulverized compound by at least a factor of 100.

21. The method of any one of clauses 1 to 20, wherein a polarization of the compound following transferring of polarization from the electron spins of the compound to the nuclear spins of the compound exceeds 0.1%.

22. The method of clause 21, wherein the polarization of the compound following transferring of polarization from the electron spins of the compound to the nuclear spins of the compound exceeds 1%.

23. A polarization method, comprising: forming a mixture of a compound and a target material; performing at least one iteration of polarization transfer, the one iteration including: polarizing nuclear spins of a species in the compound; transferring the nuclear spin polarization of the compound to nuclear spins of the target material.

24. The polarization method of clause 23, wherein transferring the nuclear spin polarization of the compound to the nuclear spins of the target material comprises: diffusing the nuclear spin polarization of the species in the compound to nuclear spins of a first species in the target material; and transferring the nuclear spin polarization of the first species in the target material to nuclear spins of a second species in the target material.

25. The polarization method of one of clauses 23 or 24, wherein: the compound includes a dopant and a source material; and polarizing nuclear spins of the species in the compound comprises: polarizing the electron spins in the dopant in excess of 10% polarization using optical radiation; transferring the electron spin polarization of the dopant to nuclear spins of the source material.

26. The method of any one of clauses 23 to 25, wherein the dopant includes at least one of pentacene, anthracene, or derivatives thereof.

27. The method of any one of clauses 23 to 26, wherein at least one of the compound or the target material comprises particles.

28. The method of clause 27, wherein the particles include at least one dimension that is smaller than 2 μm.

29. The method of any one of clauses 27 or 28, wherein the particles comprise at least one of nanocrystals or nano-rods.

30. The method of any one of clauses 27 to 29, wherein a median size of the particles in the compound is less than 1,000,000 μm3.

31. The method of any one of clauses 23 to 30, wherein the compound is polarized to a level greater than 0.1% polarization.

32. The method of clause 31, wherein the compound is polarized to a level greater than 1% polarization.

33. The method of clause 32, wherein the compound is polarized to a level greater than 10% polarization.

34. The method of any one of clauses 23 to 33, wherein the compound is a doped molecular crystal.

35. The method of any one of clauses 23 to 34, wherein: at least one of the compound or the target material comprises a liquid or a suspension of microcrystals in a liquid; and forming the mixture of the compound and the target material comprises solidifying the mixture after combining the compound and target material.

36. The method of any one of clauses 23 to 34, wherein forming the mixture of the compound and the target material comprises: seeding a melt or a solution of the compound with particles of the target material for overgrowth by the compound; or seeding a melt or a solution of the target material with particles of the compound for overgrowth by the target material.

37. The method of any one of clauses 23 to 34, wherein the mixture comprises at least one single crystal or polycrystals of: the compound crystalized around particles of the target material; or the target material crystalized around particles of the compound.

38. The method of any one of clauses 23 to 34, wherein forming the mixture of the compound and the target material comprises combining microparticles of the target material and microparticles of the compound in solid form.

39. The method of any one of clauses 23 to 38, wherein forming the mixture of the compound and the target material comprises adding a mediator material to the mixture to improve contact between the microparticles.

40. The method of any one of clauses 23 to 39, wherein the compound includes at least one of naphthalene, p-terphenyl, benzoic acid, anthracene, or derivatives thereof.

41. The method of clause 24, wherein the first species in the target material comprises protons, and the second species in the target material comprises a species having a lower gyromagnetic ratio than a gyromagnetic ratio of the protons.

42. The method of any one of clauses 23 to 41, wherein at least 30% of the nuclear spins of the target material are within at most 10 μm distance from nuclear spins of the compound.

43. The method of any one of clauses 23 to 42, wherein a polarization of the target material, following the at least one iteration of polarization transfer, exceeds 0.1%.

44. The method of clause 43, wherein the polarization of the target material, following the at least one iteration of polarization transfer, exceeds 1%.

45. A polarization method, comprising: forming a mixture of a compound and a target material, wherein the compound includes a dopant selected to have, under optical radiation, electron spins exceeding 10% polarization, and wherein at least one of the compound or the target material is in a form of a nanostructure, wherein nuclear spins of the compound are polarized at a level of more than 0.1% polarization; and transferring polarization of the nuclear spins of the compound to the target material.

46. The method of clause 45, wherein the form of nanostructure includes at least one dimension that is smaller than 2 μm.

47. The method of any one of clauses 45 to 46, wherein the compound is in the form of microparticles or nano particles with a median size no larger than about 1,000,000 μm3.

48. The method of any one of clauses 45 to 47, wherein: the compound further includes a source material; and further comprising optically polarizing electron spins of the dopant and transferring polarization of the electron spins of the dopant to nuclear spins of the source material.

49. The method of any one of clauses 45 to 48, wherein the compound is a doped molecular crystal.

50. The method of any one of clauses 45 to 49, wherein the target material comprises a liquid, suspension of microcrystals, or solution and the method further comprises solidifying the mixture.

51. The method of any one of clauses 45, 48, or 49, wherein the compound comprises a liquid, suspension of microcrystals, or solution and the method further comprises solidifying the mixture.

52. The method of any one of clauses 45 to 49 or 51, wherein within the mixture, the target material is in a microcrystal form within at least one of a liquid, a glassy matrix, or crystalline matrix, and wherein the target material is in contact with the compound.

53. The method of clause 45, wherein the target material is in a nano-crystal form and is configured to serve as a seed for overgrowth by the compound.

54. The method of clause 53, wherein forming a mixture includes introducing the target material into a solution, a melt or a gas which includes molecules of the compound, enabling the compound to crystalize around the target material.

55. The method of clause 45, wherein forming a mixture includes combining microparticles of the target material and microparticles of the compound in a solid form.

56. The method of any one of clauses 45 to 55, further comprising: adding a mediator material to the mixture to improve contact between the microparticles; and solidifying the mixture by cooling the mixture.

57. The method of any one of clauses 45 to 56, wherein the source material includes at least one of naphthalene, p-terphenyl, benzoic acid, anthracene, or derivatives thereof.

58. The method of any one of clauses 45 to 57, wherein the dopant includes at least one of pentacene, anthracene, or derivatives thereof.

59. The method of any one of clauses 45 to 58, further comprising transferring polarization within the target material from protons to nuclear spins having a lower gyromagnetic ratio than a gyromagnetic ratio of the protons.

60. The method of any one of clauses 45 to 59, wherein at least 30% of nuclear spins of the target material are within at most 10 μm distance from nuclear spins of the compound.

61. The method of clause 45, wherein the target material or the compound comprises micro- or nanoparticles.

62. The method of clause 45, wherein the compound comprises a single crystal, polycrystal, or amorphous solid and the mixture includes particles of the target material entrapped in the compound.

63. The method of clause 45, wherein the target material comprises a single crystal, polycrystal, or amorphous solid and the mixture includes particles of the compound entrapped in the target material.

64. The method of clause 45, wherein the mixture includes particles of the target material, each particle individually entrapped in a single crystal or polycrystal of the compound.

65. The method of clause 45, wherein the mixture includes particles of the compound, each particle individually entrapped in a single crystal or polycrystal of the target material.

66. The method of clause 45, wherein the target material comprises a solution, and the mixture includes a suspension of the compound in the solution of the target material.

67. The method of any one of clauses 45 to 66, wherein nuclear spins of the compound are polarized at a level of more than 1% polarization.

68. The method of clause 67, wherein nuclear spins of the compound are polarized at a level of more than 10% polarization.

69. A system, comprising: a first housing containing: a first cavity configured to hold a pulverized compound with pre-polarized nuclear spins; a mixing apparatus configured to mix the pulverized compound into a mixture; and a first magnetic field generator configurable to maintain a magnetic field of at least 10 gauss within a predetermined portion of the first cavity during the mixing of the pulverized compound into the mixture.

70. The system of clause 69, wherein the first housing further contains: a port for introducing a material to the first cavity.

71. The system of any one of clauses 69 to 70, wherein the material comprises: a first solvent or a combination of the first solvent and a target material.

72. The system of any one of clauses 69 to 71, wherein the system further comprises: a second housing containing: a second cavity configured to hold a compound with pre-polarized nuclear spins; a pulverizer configured to pulverize the compound into the pulverized compound, the pulverized compound comprising pieces having a median size of no greater than 1 mm3; and a second magnetic field generator configurable to maintain a magnetic field of at least 10 gauss within a predetermined portion of the second cavity during the pulverization of the compound.

73. The system of clause 72, wherein the first housing and the second housing are the same housing, and the first cavity and the second cavity are the same cavity.

74. The system of any one of clauses 69 to 73, wherein the system further comprises: a third housing containing: a third cavity configured to hold the mixture; and a third magnetic field generator configurable to maintain a magnetic field of at least 10 gauss within a predetermined portion of the third cavity during the pulverization of the compound; a cooler configurable to cool a mixture in the third cavity to a predetermined temperature of minus 20 degrees Celsius or lower within 60 sec.

75. The system of clause 74, wherein the cooler contains a reservoir for holding liquid nitrogen.

76. The system of any one of clauses 74 to 75, wherein the first housing and the third housing are the same housing, and the first cavity and the third cavity are the same cavity.

77. The system of any one of clauses 69 to 76, wherein the system further comprises: a radiofrequency generator; and a fourth housing containing: a fourth cavity configured to hold the mixture; a fourth magnetic field generator configurable to maintain a magnetic field of at least 10 gauss having inhomogeneities of at most ±20% within a predetermined portion of the fourth cavity; and radiofrequency coils connected to the radiofrequency generator and configured to produce two or more electromagnetic fields at two or more frequencies that excite nuclear spins in the mixture.

78. The system of clause 77, wherein the first housing and the fourth housing are the same housing, and the first cavity and the fourth cavity are the same cavity.

79. The system of any one of clauses 69 to 78, wherein the system further comprises: a fifth housing containing: a fifth cavity configured to hold the mixture; and a fifth magnetic field generator configurable to maintain a magnetic field of at least 10 gauss within a predetermined portion of the fifth cavity during the pulverization of the compound; and a port for introducing a second solvent having a temperature greater than 0 degrees to the fifth cavity.

80. The system of clause 79, wherein the fifth housing further comprises: a filter configured to separate the compound from the target material.

81. The system of any one of clauses 69 to 80, wherein the first housing and the fifth housing are the same housing, and the first cavity and the fifth cavity are the same cavity.

82. The system of any one of clauses 69 to 81, wherein the first housing further comprises: a conveyor configured to convey the first cavity through a location within 1 second or less; and the magnetic field at the location is lower than 400 gauss during the conveying of the first cavity through the location.

83. The system of any one of clauses 69 to 82, wherein the first magnetic field generator configurable to maintain a magnetic field of at least 500 gauss within a predetermined portion of the first cavity.

84. The system of any one of clauses 69 to 83, wherein the second magnetic field generator configurable to maintain a magnetic field of at least 500 gauss within a predetermined portion of the second cavity.

85. The system of any one of clauses 74 to 76, wherein the third magnetic field generator configurable to maintain a magnetic field of at least 500 gauss within a predetermined portion of the third cavity.

86. The system of any one of clauses 77 to 78, wherein the fourth magnetic field generator configurable to maintain a magnetic field of at least 500 gauss within a predetermined portion of the fourth cavity.

87. The system of any one of clauses 79 to 81, wherein the fifth magnetic field generator configurable to maintain a magnetic field of at least 500 gauss within a predetermined portion of the fifth cavity.

88. A method, comprising: introducing into a first cavity a pulverized compound with pre-polarized nuclear spins; mixing the pulverized compound into a mixture; and

wherein a magnetic field of at least 10 gauss is maintained within the first cavity during the mixing of the pulverized compound into the mixture.

89. The method of clause 88, wherein: the method further comprises introducing into the first cavity a first solvent or a combination of the first solvent and a target material; and mixing the pulverized compound into a mixture comprises mixing the pulverized compound with the first solvent or combination of the first solvent and a target material.

90. The method of any one of clauses 88 to 89, wherein the method further comprises: pulverizing, in a second cavity, a compound with pre-polarized nuclear spins into the pulverized compound, the pulverized compound comprising pieces having a median size of no greater than 1 mm3; and maintaining within the second cavity a magnetic field of at least 10 gauss during the pulverization of the compound.

91. The method of any one of clauses 88 to 90, wherein the method further comprises: cooling the second cavity to a temperature of minus 20 degrees Celsius or lower during pulverization.

92. The method of any one of clauses 88 to 91, wherein cooling the second cavity comprises introducing a coolant to the second cavity.

93. The method of any one of clauses 88 to 92, wherein the method further comprises: cooling, in a third cavity, the mixture to a predetermined temperature of minus 20 degrees Celsius or lower within 60 sec by introducing a coolant to the third cavity; and maintaining a magnetic field of at least 10 gauss within a predetermined portion of the third cavity during the cooling of the third cavity.

94. The method of clause 93, wherein the coolant is liquid nitrogen.

95. The method of any one of clauses 88 to 94, wherein the method further comprises: applying to the mixture, in a fourth cavity for a predetermined duration, two or more electromagnetic fields at two or more frequencies that excite nuclear spins in the mixture, and a magnetic field of at least 10 gauss having inhomogeneities of at most ±20% within a predetermined portion of the fourth cavity.

96. The method of any one of clauses 88 to 95, wherein the method further comprises: introducing into a fourth cavity containing the mixture through a port, a second solvent having a temperature greater than 0 degrees, thereby dissolving from the mixture the target material; and maintaining a magnetic field of at least 10 gauss within a predetermined portion of the fourth cavity during introduction of the mixture.

97. The system of any one of clauses 88 to 96, wherein a magnetic field at least 500 gauss is maintained within a predetermined portion of the first cavity.

98. The system of any one of clauses 90 to 97, wherein a magnetic field at least 500 gauss is maintained within a predetermined portion of the second cavity.

99. The system of any one of clauses 93 to 98, wherein a magnetic field at least 500 gauss is maintained within a predetermined portion of the third cavity.

100. The system of any one of clauses 95 to 99, wherein a magnetic field at least 500 gauss is maintained within a predetermined portion of the fourth cavity.

101. The method of any one of clauses 88 to 100, wherein the method further comprises: conveying a sample of the mixture through a location within 1 second; and the magnetic field at the location is between 0.1 and 400 gauss during the conveying of the sample through the location.

102. A method for preparing a target material, the method comprising: introducing into a cavity, a compound with pre-polarized nuclear spins; introducing into the cavity, material comprising a solvent or a combination of a solvent and target material; pulverizing the compound, the pulverized compound comprising pieces having a median size of no greater than 1 mm3; mixing the pulverized compound and the materials into a mixture; wherein the temperature of the cavity is maintained at less than −20 degree C. and a magnetic field of at least 10 gauss is applied to the cavity during the pulverizing and mixing of the compound; polarizing the mixture for a predetermined duration by: 1) applying to the mixture, in the cavity for a predetermined duration, two or more electromagnetic fields at two or more frequencies that excite nuclear spins in the mixture, and a magnetic field of at least 10 gauss having inhomogeneities of at most ±20% within a predetermined portion of the fourth cavity; or 2) conveying the mixture through a location within 1 second, wherein a magnetic field at the location is less than 300 gauss during the conveying of the sample through the location; introducing a second solvent having a temperature greater than 0 degree C. into the cavity having, thereby dissolving from the mixture the target material; and extracting the target material from the cavity.

103. The method of clause 102, wherein a magnetic field at least 500 gauss is applied to the cavity.

104. A method of forming an NMR or MRI target material, the method comprising: obtaining at least 0.1 mg of a compound containing nuclear spins, wherein the nuclear spins in the compound exceed 0.1% polarization; exposing the compound to a target material; and mechanically altering the compound to increase a surface area of the compound and facilitate transfer of polarization from the compound to the target material.

105. The method of clause 104, wherein nuclear spin polarization in the target material after the transfer of polarization from the compound exceeds 0.1% polarization.

106. The method of any one of clauses 104 to 105, wherein the compound is selected to have, under optical radiation, electron spins exceeding 10% polarization.

107. The method of any one of clauses 104 to 106, wherein the compound includes a mixture of a dopant and an additional material, and wherein the dopant is selected to have, under optical radiation, electron spins exceeding 10% polarization.

108. The method of any one of clauses 104 to 107, further comprising optically hyperpolarizing electron spins in the compound; and transferring polarization from the electron spins of the compound to nuclear spins of the compound, at least in part by exposing the compound to a magnetic field.

109. The method of any one of clauses 104 to 108, wherein the compound includes a doped molecular crystal.

110. The method of clause 109, wherein the molecular crystal includes at least one of naphthalene, p-terphenyl, benzoic acid, or derivatives thereof.

111. The method of any one of clauses 109 to 110, wherein the dopant includes at least one of pentacene, anthracene, or derivatives thereof.

112. The method of any one of clauses 104 to 111, wherein: a magnetic field of at least 5 gauss is applied to the compound during the transfer of polarization to the target material; and the compound is exposed to the target material before the transfer of polarization to the nuclear spins of the compound.

113. The method of any one of clauses 104 to 111, wherein the compound is exposed to the target material after mechanically altering the compound.

114. The method of clause 104, further comprising applying a magnetic field of at least 10 gauss to the compound after exposure to the target material and during the mechanical alteration.

115. The method of clause 104, wherein: the target material comprises at least one of a liquid or a solute in a solution; exposing the compound to a target material comprises mixing the mechanically altered compound and the target material; and the method further comprises freezing the mixture of the mechanically altered compound and the target material.

116. The method of clause 104, wherein both the target material and the mechanically altered hyperpolarized compound are in microcrystalline form.

117. The method of clause 116, wherein the method further comprises adding to the exposed compound at least one of a liquid or an amorphous material to facilitate polarization transfer between the compound and the target material.

118. The method of any one of clauses 104 to 117, wherein the target material comprises at least one of urea, pyruvic acid, pyruvates, fumarate, bicarbonate, dehydroascorbate, glutamine, acetate, alpha-ketoglutarate, dihydroxyacetone, acetoacetate, lactate, glucose, ascorbic acid, zymonic acid, or derivatives thereof.

119. The method of clause 104, wherein: exposing the compound to the target material comprises forming a mixture of the compound and the target material; and the method further comprises separating the target material from the compound and injecting the target material into biological tissue.

120. The method of clause 108, wherein the compound is in a form of molecular crystals, and wherein the transfer of polarization from the optically polarized electron spins further includes exposing the molecular crystals to microwave energy.

121. The method of any one of clauses 104 to 120, wherein the magnetic field is tuned to match a value for which an energy level of the optically hyperpolarized electron spins and the nuclear spins of the compound share a common resonance.

122. The method of clause 104, wherein the mechanically altering reduces the compound to at least one of micro particles or nano particles with a median size no larger than 1,000,000 μm3.

123. The method of any one of clauses 104 to 122, wherein the polarization transfer from the nuclear spins of the mechanically altered compound to the nuclear spins of the target material occurs via spin-diffusion by nuclei of a common species.

124. The method of clause 123, further comprising transferring polarization within the target material from protons to nuclear spins having a lower gyromagnetic ratio than a gyromagnetic ratio of the protons.

125. The method of clause 104, wherein the polarization of the nuclear spins of compound occurs by dynamic nuclear polarization from electron spins at temperatures below 4K.

126. The method of clause 114, further comprising applying a magnetic field of at least 500 gauss to the compound after exposure to the target material and during the mechanical alteration.

127. The method of any one of clauses 104 to 126, wherein nuclear spins in the target material after the transfer of polarization from the compound exceed 1% polarization.

128. The method of clause 127, wherein nuclear spins in the target material after the transfer of polarization from the compound exceed 10% polarization.

129. A method of transferring polarization, comprising: hyperpolarizing a compound at a first location, the hyperpolarized compound having a relaxation time greater than 2.5 hours when maintained at a temperature between 70 and 273 Kelvin in a magnetic field of a strength between 0.05 and 4 Tesla; transporting the hyperpolarized compound to a second location in a container configured to maintain the hyperpolarized compound at the temperature in the magnetic field strength; and transferring polarization from the compound to a target material at the second location.

130. The method of clause 129, wherein the compound is a crystalline compound.

131. The method of any one of clauses 129 to 130, wherein the second location is more than a kilometer from the first location.

132. The method of any one of clauses 129 to 131, wherein a duration of the transportation is greater than an hour.

133. The method of any one of clauses 129 to 132, wherein the container is a dry shipping container including a refrigerant and an absorption material.

134. The method of any one of clauses 129 to 133, wherein the container includes a Dewar, a magnetic field source, and a magnetic shield for substantially containing the magnetic field within the shipping container.

135. The method of any one of clauses 129 to 134, wherein transporting the hyperpolarized compound to the second location in the container comprises automatically monitoring the magnetic field and the temperature within the shipping container.

136. The method of any one of clauses 129 to 135, wherein the temperature is less than 150 K and the magnetic field strength is between 0.3 and 1.5 tesla.

137. The method of any one of clauses 129 to 136, wherein the target material is a contrast agent.

138. The method of any one of clauses 129 to 137, wherein the compound is a doped molecular crystal.

139. The method of any one of clauses 129 to 138, wherein the doped molecular crystal includes at least one of naphthalene, p-terphenyl, benzoic acid, or derivatives thereof.

140. The method of any one of clauses 129 to 139, wherein the dopant includes at least one of pentacene, anthracene, or derivatives thereof.

141. The method of any one of clauses 129 to 140, wherein a polarization of the compound following hyperpolarization exceeds 0.1%.

142. The method of clause 141, wherein the polarization of the compound following hyperpolarization exceeds 1%.

143. A container, comprising: a refrigerant; a magnetic field source; a cryostat containing a hyperpolarized compound having a relaxation time greater than 2.5 hours when maintained at a temperature between 70 and 273 Kelvin in a magnetic field of a strength between 0.1 and 4 Tesla; and wherein the container is configured to maintain the hyperpolarized compound at the temperature in the magnetic field using the refrigerant and the magnetic field source.

144. The container of clause 143, wherein the container is configured to maintain the hyperpolarized compound at the temperature in the magnetic field for more than an hour.

145. The container of any one of clauses 143 to 144, wherein the container further includes a sensor configured to automatically monitor the magnetic field and the temperature.

146. The container of any one of clauses 143 to 145, wherein the container is configured to provide an alert when a temperature criterion or a magnetic field strength criterion are satisfied.

147. The container of any one of clauses 143 to 146, wherein the hyperpolarized compound is a crystalline compound.

148. The container of any one of clauses 143 to 147, wherein the hyperpolarized compound is a doped molecular crystal.

149. The container of clause 148, wherein the doped molecular crystal includes at least one of naphthalene, p-terphenyl, benzoic acid, or derivatives thereof.

150. The container of any one of clauses 148 to 149, wherein the dopant includes at least one of pentacene, anthracene, or derivatives thereof.

151. The container of any one of clauses 148 to 150, wherein the relaxation time of the hyperpolarized compound is greater than 5 hours when maintained at the temperature in the magnetic field.

152. The container of any one of clauses 148 to 151, wherein the container further comprises a magnetic shield for substantially containing the magnetic field within the shipping container.

153. The container of any one of clauses 148 to 152, wherein a polarization of the hyperpolarized compound exceeds 0.1%.

154. The container of clause 153, wherein a polarization of the hyperpolarized compound exceeds 1%.

155. A method of manufacturing a hyperpolarized biocompatible material, the method comprising: mixing a hyperpolarized biocompatible material with a non-biocompatible material containing nuclear spins into a mixture, wherein the non-biocompatible material includes a dopant with hyperpolarizable electron spins; optically hyperpolarizing the electron spins of the dopant; transferring polarization from the electron spins of the dopant to the nuclear spins of the non-biocompatible material; transferring polarization of the nuclear spins of the non-biocompatible material to nuclear spins of the biocompatible material; and preparing a second mixture of the biocompatible material for injection into biological tissue at least in part by separating the second mixture from the first mixture, the second mixture including at least some of the biocompatible material from the first mixture and having a concentration of less than 1 mM of the non-biocompatible material from the first mixture.

156. The method of clause 155, wherein separating at least some of the biocompatible material from the mixture comprises: differentially dissolving the biocompatible material and the non-biocompatible material into a solution using a solvent; and separating the solution from the mixture.

157. The method of clause 156, wherein the solution is separated from the mixture using a filter.

158. The method of clause 157, wherein the filter has a pore size less than or equal to 200 nanometers.

159. The method of any one of clauses 155 to 158, wherein: a polarity of the non-biocompatible material differs from a polarity of the biocompatible material; and separating at least some of the biocompatible material from the mixture further comprises separating biocompatible material dissolved in the solution from non-biocompatible material dissolved in the solution using the difference in polarity.

160. The method of clause 159, wherein the biocompatible material dissolved in the solution is separated from the non-biocompatible material dissolved in the solution using reversed-phase chromatography.

161. The method of clause 156, wherein: the biocompatible material has a greater solubility in the solvent than the non-biocompatible material.

162. The method of clause 156, wherein: the solvent dissolves the non-biocompatible material and does not dissolve the biocompatible material.

163. The method of clause 156, wherein separating at least some of the biocompatible material from the mixture comprises: dissolving the mixture in a combination of an organic solvent and an aqueous solvent, the biocompatible material preferentially dissolving in the aqueous solvent to form an aqueous solution and the non-biocompatible material preferentially dissolving in the organic solvent to form an organic solution; and separating the aqueous solution from the organic solution.

164. The method of any one of clauses 155 to 163, wherein the non-biocompatible material is a molecular crystal.

165. The method of any one of clauses 155 to 164, wherein transferring polarization from the electron spins of the dopant to the nuclear spins of the non-biocompatible material comprises exposing the non-biocompatible material to a magnetic field.

166. The method of any one of clauses 155 to 165, wherein a polarization of the hyperpolarized biocompatible material exceeds 0.1%.

167. The method of clause 166, wherein a polarization of the hyperpolarized biocompatible material exceeds 1%.

168. A method of forming an NMR or MRI target material, the method comprising: obtaining at least 0.1 mg of a compound containing nuclear spins, wherein the compound is hyperpolarized at a level of more than 0.1% polarization; creating a mixture containing the compound and a target material by dissolving the compound in a solution; and freezing the mixture of the solution and the target material within a predetermined time from the beginning of the mixing of the compound and target material.

169. The method of clause 168, wherein the predetermined time is between 5 and 20 seconds.

170. The method of any one of clauses 168 to 169, wherein creating the mixture comprises co-dissolving the compound with the target material.

171. The method of any one of clauses 168 to 169, wherein creating the mixture comprises suspending nanoparticles of the target material in the solution.

172. The method of any one of clauses 168 to 171, wherein the compound is selected to have, under optical radiation, electron spins exceeding 10% polarization.

173. The method of any one of clauses 168 to 172, wherein the compound contains a dopant which is selected to have, under optical radiation, electron spins exceeding 10% polarization.

174. The method of any one of clauses 168 to 173, wherein obtaining the compound containing nuclear spins further comprises: obtaining the compound; optically hyperpolarizing electron spins in the compound; and transferring polarization from the electron spins of the compound to nuclear spins of the compound, the transferring including exposing the compound to the magnetic field.

175. The method of any one of clauses 168 to 174, wherein the compound is polarized at a level of more than 0.1% polarization.

176. The method of any one of clauses 168 to 175, wherein the compound is polarized at a level of more than 1% polarization.

Other embodiments will be apparent from consideration of the specification and practice of the embodiments disclosed herein. It is intended that the specification and examples be considered as an example only, with a true scope and spirit of the disclosed embodiments being indicated by the following claims.

1-128. (canceled)

129. A method of transferring polarization, comprising:
hyperpolarizing a compound at a first location to create a hyperpolarized compound, the hyperpolarized compound having a relaxation time greater than 2.5 hours when maintained at a temperature between 70 and 273 Kelvin in a magnetic field of a strength between 0.05 and 4 Tesla;
transporting the hyperpolarized compound to a second location in a container configured to maintain the hyperpolarized compound at the temperature in the magnetic field strength; and
transferring polarization from the hyperpolarized compound to a target material at the second location.

130. The method of claim 129, wherein the compound is a crystalline compound.

131. The method of claim 129, wherein the second location is more than a kilometer from the first location.

132. The method of claim 129, wherein a duration of the transportation is greater than an hour.

133. The method of claim 129, wherein the container is a dry shipping container including a refrigerant and an absorption material.

134. The method of claim 129, wherein the shipping container includes a Dewar, a magnetic field source, and a magnetic shield for substantially containing the magnetic field within the shipping container.

135. The method of claim 129, wherein transporting the hyperpolarized compound to the second location in the container comprises automatically monitoring the magnetic field and the temperature within the shipping container.

136. The method of claim 129, wherein the temperature is less than 150 K and the magnetic field strength is between 0.3 and 1.5 tesla.

137. The method of claim 129, wherein the target material is a contrast agent.

138. The method of claim 129, wherein the compound is a doped molecular crystal.

139. The method of claim 138, wherein the doped molecular crystal includes at least one of naphthalene, p-terphenyl, benzoic acid, or derivatives thereof.

140. The method of claim 138, wherein the dopant includes at least one of pentacene, anthracene, or derivatives thereof.

141-176. (canceled)

177. The method of claim 129, wherein the method further comprises:
reducing a concentration of paramagnetic impurities in the hyperpolarized compound following the hyperpolarization of the compound.

178. The method of claim 177, wherein reducing the concentration of the paramagnetic impurities comprises:
increasing the temperature above a threshold, thereby reducing the concentration of the paramagnetic impurities and increasing the relaxation time of the compound.

179. The method of claim 177, wherein the concentration of paramagnetic impurities is reduced from a concentration of more than 10 ppm.

180. The method of claim 177, wherein the concentration of paramagnetic impurities is reduced to a concentration of less than 1 ppm.

181. The method of claim 129, wherein:
the compound comprises transient paramagnetic impurities; and
the compound is hyperpolarized using the transient paramagnetic impurities.

182. The method of claim 181, wherein the method further comprises applying optical radiation to:
create the transient paramagnetic impurities; or
hyperpolarize the compound.

183. The method of claim 181, wherein the transient paramagnetic impurities comprise radicals or paramagnetic defects.

184. The method of claim 181, wherein a concentration of transient paramagnetic impurities in the compound during hyperpolarization is greater than a concentration of transient paramagnetic impurities in the hyperpolarized compound during transport.
